(19) United States
(12) Patent Application Publication
(10) Pub. No.: US 2026/0041653 A1
(43) Pub. Date: Feb. 12, 2026

(54) AN HDAC INHIBITOR FOR TREATING CANCER WITH A MODIFIED STK11 ACTIVITY OR EXPRESSION

(71) Applicant: Tango Therapeutics, Inc., Boston, MA (US)

(72) Inventors: Leanne G. AHRONIAN, Cambridge, MA (US); Chengyin MIN, Wellesley, MA (US)

(21) Appl. No.: 19/101,393

(22) PCT Filed: Aug. 4, 2023
(86) PCT No.: PCT/US2023/029563

§ 371 Date: Feb. 5, 2025

Related U.S. Application Data
(60) Provisional application No. 63/395,503, filed on Aug. 5, 2022.
(60) Provisional application No. 63/422,723, filed on Nov. 4, 2022.
(60) Provisional application No. 63/490,217, filed on Mar. 14, 2023.
(60) Provisional application No. 63/496,290, filed on Apr. 14, 2023.

(51) Int. Cl. A61K 31/167; A61K 31/519; A61K 33/243; A61K 39/00; A61P 35/00; C07K 16/28; C12Q 1/6886

(52) U.S. Cl. CPC A61K 31/167; A61K 31/519; A61K 33/243; A61P 35/00; C07K 16/2818; C07K 16/2827; C12Q 1/6886; A61K 2039/505; C12Q 2600/106; C12Q 2600/156

(57) ABSTRACT

The disclosure relates to methods of treating cancers, including cancers having modified STK11 activity or expression, by administering an effective amount of a histone deacetylase (HDAC) inhibitor.

### CROSS-REFERENCE TO RELATED APPLICATIONS

This application claims priority to and the benefit of U.S. Provisional Patent Application No. 63/395,503, filed on Aug. 5, 2022, U.S. Provisional Patent Application No. 63/422,723, filed on Nov. 4, 2022, U.S. Provisional Patent Application No. 63/490,217, filed on Mar. 14, 2023, and U.S. Provisional Patent Application No. 63/496,290, filed on Apr. 14, 2023, the entire contents of which are incorporated by reference herein for all purposes.

### BACKGROUND

STK11 is a tumor suppressor gene that drives immune evasion when deleted or inactivated, and is frequently mutated in lung adenocarcinomas, such as non-small cell lung cancer (NSCLC). Identifying a drug target that could disable the immune evasion caused by an STK11 loss-of-function mutation could reverse cancer cell immune evasion and enable immune cells to eliminate the cancer cell containing an STK11 mutation.

The current standard of care for treating a lung adenocarcinoma patient typically includes administration of an anti-PD-1 therapy or an anti-PD-L1 therapy. However, lung adenocarcinoma patients having STK11 loss of function mutations respond poorly to such anti-PD-1 and anti-PD-L1 therapies. Skoulidis, F., et al Cancer Discovery 8 (7): 822-835 (2018) (DOI: 10.1158/2159-8290.CD-18-0099) and Skoulidis, F., et al Journal of Clinical Oncology 37(15): supp 102 (2019) (DOI: 10.1200/JCO.2019.37.15_suppl.102), each of which is hereby incorporated by reference in their entirety.

Attempts to improve lung adenocarcinoma patient outcomes include the development of a class of compounds called Kristen rat sarcoma 2 viral oncogene homolog (KRAS) inhibitors that have been developed to treat cancers (e.g., NSCLC) in patients having certain KRAS (e.g., KRAS^{G12C}) mutations. However, such compounds have certain limitations and there remains a need to improve patient outcomes where the current standard of care is insufficient.

Thus, there is a need to identify methods of treating cancer patients having an STK11 mutation.

### SUMMARY

Provided herein are methods of treating a subject having, or at risk of developing, a cancer, the method comprising administering to the subject an effective amount of a histone deacetylase inhibitor, wherein the cancer is identified as having modified STK11 activity or expression.

Provided herein are also methods for selecting a subject for treatment with an HDAC inhibitor the method comprising: identifying subjects having a cancer characterized by the presence of cells having modified STK11 activity or expression; and selecting thus identified subjects for treatment with an HDAC inhibitor.

Also provided are methods for ascertaining susceptibility of a subject to treatment with an HDAC inhibitor, the method comprising: determining: i) the presence or absence of a STK11 mutation; and/or ii) the level of STK11 activity or expression in the subject or a sample derived from the subject; wherein the presence of a STK11 mutation and/or a modified level of STK11 activity or expression is indicative of susceptibility to treatment with an HDAC inhibitor.

### DESCRIPTION OF THE DRAWINGS

FIG. 1A depicts the tumor growth curve for mice bearing MC38_sgSTK11 tumors treated with a control antibody anti-IgG2a (10 mg/kg i.p. BIW), a combination of Compound I (3, 10, 30, 75 and 150/100 mg/kg p. o., QD)+the control antibody anti-IgG2a (10 mg/kg i.p. BIW), an anti-PD1 inhibitor (10 mg/kg i.p., BIW), and a combination of Compound I (3, 10, 30, 75 and 150/100 mg/kg p.o., QD)+anti-PD1 inhibitor (10 mg/kg i.p., BIW). The data is plotted according to the groups described in Example 1.

FIG. 1B depicts the tumor growth curve in the MC38 STK11 knockout mouse model treated with either anti-IgG2a (10 mg/kg i.p., BIW), Anti-PD1 (10 mg/kg i.p., BIW), Compound I (30 mg/kg p.o., QD), or Anti-PD1 (10 mg/kg i.p., BIW)+Compound I (30 mg/kg p.o., QD) was monitored over the course of treatment and plotted by individual animal.

FIG. 2A depicts the survival curve for mice bearing MC38_sgSTK11 tumors treated with a control antibody anti-IgG2a (10 mg/kg i.p., BIW), a combination of Compound I (3, 10, 30, 75 and 150/100 mg/kg p.o., QD)+the control antibody anti-IgG2a (10 mg/kg i.p. BIW), or an anti-PD1 inhibitor (10 mg/kg i.p., BIW). The data is plotted according to the groups described in Example 1.

FIG. 2B depicts the survival curve for mice bearing MC38_sgSTK11 tumors treated with a control antibody anti-IgG2a (10 mg/kg i.p., BIW), a combination of Compound I (3, 10, 30, 75 and 150/100 mg/kg p.o. QD)+anti-PD1 inhibitor (10 mg/kg i.p., BIW), or an anti-PD1 inhibitor (10 mg/kg i.p., BIW) The data is plotted according to the groups described in Example 1.

FIG. 2C shows a survival plot of mice with STK11-deleted MC38 tumors treated with Anti-IgG2a 10 mg/kg, i.p., BIW, Anti-IgG2a 10 mg/kg, i.p., BIW+Compound I, 30 mg/kg, p.o., QD, Anti-PD1 10 mg/kg, i.p., BIW, or Anti-PD1 10 mg/kg, i.p., BIW+Compound I, 30 mg/kg, p.o., QD as indicated.

FIG. 3A depicts the tumor growth curve for untreated control mice and mice that had survived treatment with either a combination of Compound I (75 mg/kg, p.o., QD)+a control antibody anti-IgG2a (10 mg/kg i.p. BIW) or a combination of an exemplary HDAC inhibitor (3, 10, 30, 75 and 150/100 mg/kg)+anti-PD1 inhibitor (10 mg/kg, i.p., BIW) and were rechallenged with MC38_sgSTK11 implants as described in Example 2.

FIG. 3B shows a plot of the tumor volume in mice that were re-challenged with STK11-deleted MC38 tumors as described in Example 2 (combined in a single group) in parallel with a control group of previously untreated mice. All animals remained off-treatment, and tumor size was plotted over time after re-challenge.

FIG. 4 depicts the tumor growth curve for mice bearing MC38_sgSTK11 tumors treated with a control antibody anti-IgG2a (10 mg/kg i.p. BIW), Compound I (30 mg/kg, p.o. QD), an anti-PD1 inhibitor (10 mg/kg i.p., BIW) and a combination of Compound I (30 mg/kg p.o., QD) and anti-PD1 inhibitor (10 mg/kg i.p., BIW). The mice with complete tumor regression in the initial experiment were rechallenged at day 69 with MC38_sgSTK11 implants.

FIG. 5A shows a volcano plot of an unbiased in vivo CRISPR screen identifying HDAC1 knockout as a sensitizer to anti-PD1 in STK11-deleted MC38 tumors.

FIG. 5B shows waterfall plots of the Project Achilles CRISPR scores for HDAC1, HDAC2, and HDAC3 in a panel of cell lines. Negative scores indicate depletion of cells with knockout of the indicated gene.

FIG. 6A shows a graph of a dose-dependent binding of Compound I to HDAC1, by cellular NanoBRET target engagement assay.

FIG. 6B show graphs of a dose-dependent binding of Compound I to HDAC2, by cellular NanoBRET target engagement assay.

FIG. 6C show graphs of a dose-dependent binding of Compound I inhibitor to HDAC3 by cellular NanoBRET target engagement assay.

FIG. 7A depicts the tumor growth curve in the CT26 STK11 knockout mouse model treated with either control antibody Anti-IgG2a (10 mg/kg, i.p., BIW), Anti-IgG2a (10 mg/kg, i.p., BIW)+Compound I (75 mg/kg, p.o., QD), Anti-PD1 (10 mg/kg, i.p., BIW), or (Anti-PD1 10 mg/kg, i.p., BIW)+Compound I (75 mg/kg, p.o., QD). The tumor volume was monitored over the course of treatment and plotted by individual animal. STK11 knockout renders CT26 tumors resistant to anti-PD1 treatment.

FIG. 7B shows a survival plot of mice with STK11-deleted CT26 tumors treated with control antibody Anti-IgG2a (10 mg/kg, i.p., BIW), Anti-IgG2a (10 mg/kg, i.p., BIW)+Compound I (75 mg/kg, p.o., QD), Anti-PD1 (10 mg/kg, i.p., BIW), or Anti-PD1 (10 mg/kg, i.p., BIW)+Compound I (75 mg/kg, p.o., QD) as indicated.

FIG. 8A depicts the tumor growth curve of STK11-deficient MC38 tumor cells in C57BL/6 animals and athymic BALB/c Nude mice treated with Anti-IgG2a (10 mg/kg, i.p., BIW). Anti-IgG2a (10 mg/kg, i.p., BIW)+Compound I (30 mg/kg, p.o., QD), Anti-PD1 10 mg/kg, i.p., BIW, or Anti-PD1 10 mg/kg, i.p., BIW+Compound I, 30 mg/kg, p.o., QD as indicated.

FIG. 8B depicts the tumor growth curve of STK11-deficient MC38 tumor cells in C57BL/6 animals and athymic BALB/c Nude mice treated with Anti-IgG2a (10 mg/kg, i.p., BIW). Anti-IgG2a (10 mg/kg, i.p., BIW)+Compound I (75 mg/kg, p.o., QD), Anti-PD1 (10 mg/kg, i.p., BIW), or Anti-PD1 (10 mg/kg, i.p., BIW)+Compound I (75 mg/kg, p.o., QD) as indicated.

FIG. 9A shows a graph depicting the change in gene expression for CXCL9, 10, and 11 as measured by Nanostring PanCancer IO 360 in STK11−/− MC38 tumors treated for 7 days with 30 mg/kg of Compound I or anti-PD1 alone or in combination.

FIG. 9B shows a graph depicting the change in gene expression for CCL1 and CCL22 as measured by Nanostring PanCancer IO 360 in STK11−/− MC38 tumors treated for 7 days with 30 mg/kg of Compound I or anti-PD1 alone or in combination.

FIG. 9C shows a graph depicting the change in gene expression for HLA genes as measured by Nanostring PanCancer IO 360 in STK11−/− MC38 tumors treated for 4 days with 0.2 uM of Compound I or solvent control.

FIGS. 10A-10B show graphs of TIL profiling by flow cytometry of STK11-deleted MC38 tumors treated for 7 days with 10 mg/kg of Compound I alone or in combination with anti-PD1.

FIG. 10C-10D show graphs of IFNgamma expression by a tumor (FIG. 10C) or in co-culture of human NSCLC cells with PBMCS and fibroblasts (FIG. 10D) treated with Compound I alone or in combination with anti-PD1 for 72 hrs.

FIG. 10E shows graphs profiling the relative abundance of All T cells and T regulatory cells of STK11-deleted MC38 tumors from mice treated for 7 days with vehicle, 10 mg/kg of Compound I alone or in combination with anti-PD1.

FIGS. 11A-11C show plots of gene expression changes in A549 cells treated with vorinostat, domatinostat, and Compound I using the PanCancer IO360 panel and the top three ranked gene ontology groups for each compound as determined from the Nanostring data in using the nSolver software.

FIG. 12A shows plots for the erythroid and myeloid cell viability after treatment Compound I at different concentrations as indicated. The efficacious dose range of Compound I is also plotted (shaded area, 3 mg/kg to 75 mg/kg).

FIG. 12B-12C depicts the tumor growth curves STK11-deleted MC38 tumors in a mouse model treated with a clinically relevant dose of vorinostat (FIG. 12B) or Compound I (FIG. 12C) alone or in combination with anti-PD1 antibody.

FIG. 12D shows a plot comparing Compound I concentrations to HDAC1 or HDAC3 inhibition in vivo. Shaded boxes indicate tolerated and efficacious dose ranges of Compound I.

FIG. 13 shows a plot of the predicted plasma concentration (ng/mL) over time upon administration to humans and the predicted window between the efficacious and non-selective dose.

FIG. 14A shows a Western blot of acetylated histone 3 lysine 9 (H3K9Ac) from mouse MC38 tumor tissue after treatment with Compound I for 7 days at the indicated dose.

FIG. 14B shows a Quantification of the H3K9Ac western blot in (E) and normalized to total histone H3.

FIG. 14C shows the plasma concentration of Compound I administered at 30 mg/kg, 100 mg/kg and 300 mg/kg QD for two days, starting 1 hour after the last dose.

FIG. 14D shows a quantification of the levels of acetyl-histone H2B by flow cytometry in PBMC samples at the prescribed time points following administration of Compound I at 30 mg/kg, 100 mg/kg and 300 mg/kg QD for two days to MC38 tumor-bearing mice

FIG. 14E shows a quantification of the levels of acetyl-histone H3B by Western Blot in tumor samples collected at the prescribed time points following administration of Compound I at 30 mg/kg, 100 mg/kg and 300 mg/kg QD for two days to MC38 tumor-bearing mice

FIG. 15A depicts the tumor growth curve in a STK11-null CT26 (KRAS G12D mutant colon cancer) syngeneic mouse model. The mice were treated with either anti-IgG2, Anti-PD1 (10 mg/kg), Compound I (75 mg/kg), or Anti-PD1 (10 mg/kg)+Compound I (75 mg/kg). Tumor volume was monitored over the course of treatment and plotted by individual animal.

FIG. 15B depicts the tumor growth curve in a STK11-null CT26 (KRAS G12D mutant colon cancer) model. Animals were treated with either anti-IgG2, Compound I (75 mg/kg), Anti-PD1 (10 mg/kg), or Anti-PD1 (10 mg/kg)+Compound I (75 mg/kg). Tumor volume was monitored over the course of treatment and plotted by treatment group.

FIG. 15C depicts the survival curve for the KRAS G12D mutant CT26-STK11 knockout syngeneic mouse model treated with either anti-IgG2, Anti-PD1 (10 mg/kg), Compound I (75 mg/kg), or Anti-PD1 (10 mg/kg)+Compound I (75 mg/kg).

FIG. 15D depicts the tumor growth curve in a wild-type/parental CT26 (KRAS G12D mutant colon cancer) model. Animals were treated with either anti-IgG2. Compound I (75 mg/kg), Anti-PD1 (10 mg/kg), or Anti-PD1 (10 mg/kg)+Compound I (75 mg/kg). Tumor volume was monitored over the course of treatment and plotted by treatment group

FIG. 15E depicts the tumor growth curve in a wild-type/parental CT26 (KRAS G12D mutant colon cancer) model. Animals were treated with either anti-IgG2. Compound I (75 mg/kg), Anti-PD1 (10 mg/kg), or Anti-PD1 (10 mg/kg)+Compound I (75 mg/kg). Tumor volume was monitored over the course of treatment and plotted by individual animal for group 1 and group 4.

FIG. 16A depicts the tumor growth curve in a wild-type/parental CT26 (KRAS G12D mutant colon cancer) model. Animals were treated with either anti-IgG2. Compound I (75 mg/kg), Anti-CTL4A (10 mg/kg), or Anti-CTL4A (10 mg/kg)+Compound I (75 mg/kg). Tumor volume was monitored over the course of treatment and plotted by treatment group

FIG. 16B depicts the tumor growth curve in a wild-type/parental CT26 (KRAS G12D mutant colon cancer) model. Animals were treated with either anti-IgG2. Compound I (75 mg/kg), Anti-CTL4A (10 mg/kg), or Anti-CTL4A (10 mg/kg)+Compound I (75 mg/kg). Tumor volume was monitored over the course of treatment and plotted by individual animal for group 1 and group 4.

FIG. 16C depicts the tumor growth curve in a STK11-null CT26 (KRAS G12D mutant colon cancer) model treated with either anti-IgG2, Compound I (75 mg/kg), Anti-CTL4A (10 mg/kg), or Anti-CTL4A (10 mg/kg)+Compound I (75 mg/kg). Tumor volume was monitored over the course of treatment and plotted by treatment group.

FIG. 16D depicts the tumor growth curve in a STK11-null CT26 (KRAS G12D mutant colon cancer) model treated with either anti-IgG2, Compound I (75 mg/kg), Anti-CTL4A (10 mg/kg), or Anti-CTL4A (10 mg/kg)+Compound I (75 mg/kg). Tumor volume was monitored over the course of treatment and plotted by individual animal for group 1 and group 4.

FIG. 17A depicts the tumor growth curve in the STK11-null 3LL model treated with either anti-IgG2, Anti-PD1 (10 mg/kg), Compound I (75 mg/kg), or Anti-PD1 (10 mg/kg)+Compound I (75 mg/kg) Tumor volume was monitored for the depicted duration and was plotted by treatment group.

FIG. 17B depicts the survival curve for the STK11-null 3LL model treated with either anti-IgG2, Anti-PD1 (10 mg/kg), Compound I (75 mg/kg), or Anti-PD1 (10 mg/kg)+Compound I (75 mg/kg) Survival is plotted by treatment group.

### DETAILED DESCRIPTION

As generally described herein, provided are methods of treating a subject having, or at risk of developing, a cancer, the method comprising administering to the subject an effective amount of a histone deacetylase inhibitor, wherein the cancer is identified as having modified STK11 activity or expression.

The disclosure herein sets forth exemplary methods, parameters and the like. It should be recognized, however, that such description is not intended as a limitation on the scope of the present disclosure but is instead provided as a description of exemplary embodiments to assist the reader as appropriate.

As used in the present disclosure, certain words and phrases (and grammatical equivalents such as conjugations, declensions, and the like) are generally intended to have the meanings defined herein unless expressly indicated otherwise or the context in which they are used indicates otherwise.

### STK11

Serine/threonine kinase 11 protein, abbreviated as STK11 and also referred to as PJS, liver kinase BI (LKB1), renal carcinoma antigen NY-REN-19 and hLKB1 protein, is a protein kinase that in humans is encoded by the STK11 gene (HGNC symbol STK11, Ensembl ID ENSG00000118046.16). As described in Koenig, M. et al Cancer Research (2021) 81 (16): 4194-4204 (doi: 10.1158/0008-5472), which is hereby incorporated by reference in its entirety: “The serine/threonine kinase LKB1 belongs to the calcium calmodulin family, which is ubiquitously expressed in several tissues and highly conserved among eukaryotes. Over the past 15 years, LKB1 has been implicated in a number of essential biological processes such as: cell cycle control, cellular energy metabolism, angiogenesis, cell polarity, and DNA damage response. The sub-cellular localization and activity of LKB1 is controlled through its interaction with STRAD and the armadillo repeat-containing mouse protein 25 (Mo25). LKB1 regulates the activity of at least 14 downstream kinases related to the AMPK family and phosphorylates other substrates including STRAD, PTEN, and p21CDKN1A. LKB1 is phosphorylated on at least eight residues, and evidence suggests that LKB1 auto-phosphorylates itself on at least four of these, whereas the other four are phosphorylated by upstream kinases. While these post-translational modifications seem not to modify its kinase activity, they are involved in the different biological responses associated with LKB1, and likely in its interactions with other partners.”

The STK11 gene is located on human chromosome 19p 13. The gene includes nine coding exons and one noncoding exon and codes for the 433 amino acid serine/threonine-protein kinase STK11 protein, which is widely expressed in all tissues (Hemminki A, et al. Nature, 1998, 18, 184-187; Alessi, D. R., et al. Annu. Rev. Biochem. 2006, 75, 137-163; Sanchez-Cespedes M. Oncogene 2007, 26, 7825-7832). Somatic mutations or deletions of the STK11 gene are present in many cancers, including, but not limited to, lung adenocarcinomas (˜15%), non-melanoma skin cancer (˜5%), cholangiocarcinomas (˜3%), ovarian carcinomas (approximately 3%) and pancreatic adenocarcinomas (˜2%) (Sanchez-Cespedes M, et al. Cancer Res 2002, 62, 3659-62; Sanchez-Vega F, et al. Cell 2018, 173, 321-337.e10; Gurumurthy S, et al. Nature 2010, 468, 659-63; Ji H, et al. Nature 2007, 448, 807-10; Gill R K, et al. Oncogene 2011, 30, 3784-3791; Gao J, et al. Sci Signal 2013, 6 (269), pl 1; Cerami E, et al. Cancer Discov 2012, 2, 401-404; Zehir A, et al. Nat Med. 2017, 23 (6), 703-713; Robinson D R, et al. Nature. 2017, 548, 297-303). STK11 mutations found in cancer include point mutations (e.g., nonsense or frame shift mutations) predicted to be deleterious and oncogenic (Chakravarty D, et al. JCO Precis Oncol. 2017, 2017) or small indels. Mutations in the STK11 gene frequently co-occur with other STK11 genomic alterations such as copy number alteration or gene deletions. These mutations and alterations result in loss of STK11 protein expression or loss of wild-type STK11 protein activity. Non-mutational mechanisms for modified expression (e.g., loss of expression) or modified activity (e.g., loss of wildtype activity) include genomic loss or promoter methylation.

As further described in Koenig, M. et al Cancer Research (2021) 81(16): 4194-4204 (doi: 10.1158/0008-5472), which is hereby incorporated by reference in its entirety: “Up to date, more than 400 unique mutations have been described for the STK11 gene, where ˜70% of these mutations promote the truncation of the protein and the other 30% represent missense mutations (COSMIC and TCGA-Bioportal). As a tumor suppressor, a number of studies have shown the contributions of the genetic loss of LKB1 to tumorigenesis. It has been demonstrated that LKB1 controls cell cycle through the transcriptional regulation of Cyclin D1 and p21CDKN1A5, where re-expression of LKB1 leads to G1 cell cycle arrest. The role of LKB1 in controlling cell metabolism through AMPK signaling has been widely documented. We know that the LKB1-AMPK axis controls lipid and glucose metabolism. and acts as a negative regulator of the Warburg effect suppressing tumor growth. LKB1 is also important in the regulation of catabolic pathways leading to the increase of glucose uptake and modulation of glycolysis or the mobilization of lipid stores by stimulating lipases, such as adipose triglyceride lipase, to release fatty acids from triglyceride stores. LKB1-AMPK-stimulated pathways also include increased turnover of macromolecules by autophagy, allowing the turnover of old and damaged molecules, or the replenishment of nutrient stores under starvation. Additionally, several investigations have suggested the role of LKB1 in regulation of physiological and pathological angiogenesis through the regulation of VEGF, MMP-2, MMP-9, bFGF, and NOX1 expression, and its participation in neurophilin-1 degradation. Studies of LKB1 loss of function have also revealed its role in cell polarity and motility through the regulation of PAK115 and the modulation of the phosphorylation status of FAK and CDC42 activation. Together, these functions contribute to the induction of epithelial mesenchymal transition (EMT) and metastasis. In addition to this, in vivo experiments have shown evidence for the contribution of LKB1 to genotoxic DNA damage response and DNA damage repair.”

It has been reported that lung adenocarcinoma patients having cancer cells with STK11 loss of function mutations respond poorly to standard of care anti-PD-1 and anti-PD-L1 therapies. Skoulidis, F., et al Cancer Discovery 8 (7): 822-835 (2018) (DOI: 10.1158/2159-8290.CD-18-0099) and Skoulidis, F., et al Journal of Clinical Oncology 37 (15): supp 102 (2019) (DOI: 10.1200/JCO.2019.37.15_suppl. 102), each of which is hereby incorporated by reference in their entirety. A pooled CRISPR-Cas9 based in vivo screen was performed and identified STK11 as an immune evasion context, where depletion of STK11 drives resistance to immune pressure in immune competent mice. Min, C., et al Cancer Research 81 (Supp. 13): 1905 (2021) (DOI: 10.1158/1538-7445.AM2021-1905), which is hereby incorporated by reference in its entirety.

STK11 mutations have been associated with low levels of T-cell inflammation and tumor PD-L1 expression. Biton J, et al. Clin Cancer Res 2018:24:5710-23, which is hereby incorporated by reference in its entirety.

Similarly, STK11 mutations in NSCLC have been associated to poor responses to other treatment modalities including anti-VEGF therapies, platinum chemotherapies and additional single agent chemotherapies. Papillon-Cavanagh S. et al. ESMO Open. 2020, 5. E000706, which is hereby incorporated by reference in its entirety.

As used herein “modified expression” (e.g., STK11 modified expression) refers to a change in the expression levels (i.e., a decrease or increase in expression levels) of a protein in a cell (e.g., a cancer cell) in comparison to a reference cell (e.g., a healthy cell). In some embodiments, increased or decreased expression levels of a protein (e.g., STK11 protein) can be assessed by determining the copy number of the gene encoding the protein (e.g., the copy number of the STK11 gene) in a patient sample (e.g., a tumor sample) and comparing the levels with those present in a control sample (e.g., a healthy tissue sample). In some embodiments, increased or decreased expression levels of a protein (e.g., STK11 protein) can be assessed by determining the level of the protein (e.g., STK11 protein) or mRNA in a patient sample (e.g., a tumor sample) and comparing the levels with those present in a control sample (e.g., a healthy tissue sample).

As used herein “modified activity” (e.g., STK11 modified activity) refers to a change in the biological activity (e.g., enzyme activity) levels (i.e., a decrease or increase in the serine/threonine kinase activity levels of STK11) of a protein in a cell (e.g., a cancer cell) in comparison to a reference cell (e.g., a healthy cell). Mutations in the gene encoding the protein (e.g., STK11 mutations) can cause the expression of protein (e.g., mutant STK11 protein) with a level of enzymatic activity that is different from the enzymatic activity of the wild-type protein.

As used herein, an “STK11 mutation” is a mutation selected from:
(i) a mutation in the nucleotide sequence encoding STK11;
(ii) a mutation in a regulatory sequence controlling expression of the nucleotide sequence encoding STK11;
(iii) a mutation in a nucleotide encoding a protein which interacts with the transcription product of the STK11 gene;
(iv) a mutation in the translation product of the STK11 gene; and
(v) a mutation in the transcription product of the STK11 gene.

In some embodiments, the STK11 mutation is a mutation selected from:
(i) a mutation in the nucleotide sequence encoding STK11;
(ii) a mutation in a regulatory sequence controlling expression of the nucleotide sequence encoding STK11; and
(iii) a mutation in a nucleotide encoding a protein which interacts with the transcription product of the STK11 gene.

In some embodiments, the STK11 mutation is a mutation in the nucleotide sequence encoding STK11. In some embodiments, the STK11 mutation is a mutation in a regulatory sequence controlling expression of the nucleotide sequence encoding STK11. In some embodiments, the STK11 mutation is a mutation in a nucleotide encoding a protein which interacts with the transcription product of the STK11 gene. In some embodiments, the STK11 mutation is a mutation in the translation product of the STK11 gene. In some embodiments, the STK11 mutation is a mutation in the transcription product of the STK11 gene.

In some embodiments, the STK11 mutation is an inactivating or loss-of function mutation.

As used herein, a “loss-of-function mutation”, also referred to as “an inactivating mutation” refers to a mutation that results in expression of a mutant protein that exhibits reduced or absent biological activity or enzymatic activity compared to wild-type protein. A loss-of-function mutation in a gene (e.g., the STK11 gene) can also result in no expression of the wild-type protein, or the expression of only a fragment of the protein that exhibits reduced or absent biological or enzymatic activity compared to a wild-type protein. The mutation can be in a DNA nucleotide sequence, mRNA sequence, or protein sequence. In some embodiments, the mutation is a DNA mutation (e.g., a substitution, deletion, insertion, truncation, splice site, translation start site, fusion, or frameshift mutation).

In some embodiments, a loss-of-function mutation (e.g., a loss-of-function STK11 mutation) is one of the following:
1) a nonsense mutation (a genetic alteration that causes the premature termination of a protein). The altered protein may be partially or completely inactivated, resulting in a change or loss of protein function;
2) a frameshift mutation (an insertion or deletion involving a number of base pairs that is not a multiple of three, which consequently disrupts the triplet reading frame of a DNA sequence). Frameshift mutations generally cause the creation of a premature termination (stop) codon, and result in a truncated protein product;
3) a splice-site mutation (a genetic alteration in the DNA sequence that occurs at the boundary of an exon and an intron (splice site)). This change can disrupt RNA splicing resulting in the loss of exons or the inclusion of introns and an altered protein-coding sequence;
4) a translation start site mutation (a mutation that disrupts the translation initiation sequence, abolishing the initiation of translation at the normal start site, resulting in loss of mRNA translation or translation of an abnormal messenger RNA (mRNA)). Translation start site mutations result in loss of protein expression or in synthesis of a protein with an abnormal amino acid sequence;
5) a recurrent somatic mutation (having at least 5 instances recorded in the Catalogue of Somatic Mutations in Cancer (COSMIC) database) (Tate J G, et al. Nucleic Acids Res (2019) 47 (D1), D941-D947);
6) a DNA fusion (a gene created by joining parts of two different genes; may be made when part of DNA from a chromosome moves to another chromosome);
7) any other mutation predicted to reduce the function of the encoded protein by the OncoKB algorithm (Chakravarty D, et al. JCO Precis Oncol. 2017, 2017) or MutationAssessor (Reva B, Antipin Y, Sander C. Nucleic acids research. 201 1: 39 (17): el 18):

In certain embodiments, the mutation is not a variant of unknown significance (a mutation for which the association with disease risk is unclear, also known as an unclassified variant, a variant of uncertain significance, or VUS (Richards S, et al. Standards and Guidelines for the Interpretation of Sequence Variants: A Joint Consensus Recommendation of the American College of Medical Genetics and Genomics and the Association for Molecular Pathology. Genet Med. 2015 May; 17 (5): 405-424).

In some embodiments, the mutation is not a germline mutation (a gene change in a reproductive cell (egg or sperm) that becomes incorporated into the DNA of every cell in the body of the offspring), identified in the dbSNP (Sherry, S. T., et al., Nucleic Acids Res, 2001, 29:308-311).

Loss of function mutations of the STK11 gene (e.g., in a cancer cell) can result in the loss of expression of STK11 protein, the expression of only a fragment of the STK11 protein or the expression of a STK11 protein with reduced or absent enzymatic activity. (e.g., no serine/threonine kinase enzymatic activity).

Non-limiting examples of STK11 mutations that are loss-of-function mutations as defined herein are listed in Table 1 (adapted from WO2022087270). The mutations include in Table 1 were predicted to have deleterious function by OncoKB or had at least 5 occurrences in COSMIC, and excluded mutations and copy number alterations of unknown significance (i.e., VUS) and germline mutations.

One of skill in the art would appreciate that many STK11 mutations are known, or are otherwise identifiable.

TABLE 1
Exemplary STK11 loss-of-function mutations

| Protein Change | Mutation type | HGVS Genomic Nomenclature |
|---|---|---|
| A198Pfs*89 | Frameshift Deletion | 19:g.1220498del |
| A206Rfs*81 | Frameshift Deletion | 19:g.1220597del |
| A347Rfs*9 | Frameshift Deletion | 19:g.1223099_1223109del |
| A43Pfs*8 | Frameshift Deletion | 19:g.1207037del |
| C132Wfs*29 | Frameshift Deletion | 19:g.1219344del |
| D176Tfs*111 | Frameshift Deletion | 19:g.1220432del |
| D208Qfs*53 | Frameshift Deletion | 19:g.1220599_1220612del |
| D229Tfs*58 | Frameshift Deletion | 19:g.1220666del |
| D23Sfs*25 | Frameshift Deletion | 19:g.1206979_1206988del |
| D327Vfs*28 | Frameshift Deletion | 19:g.1223039_1223052del |
| D53Tfs*11 | Frameshift Deletion | 19:g.1207065del |
| E120Kfs*9 | Frameshift Deletion | 19:g.1218483del |
| E57Kfs*7 | Frameshift Deletion | 19:g.1207077del |
| F148Sfs*13 | Frameshift Deletion | 19:g.1219389del |
| F204Sfs*83 | Frameshift Deletion | 19:g.1220592del |
| F264Rfs*22 | Frameshift Deletion | 19:g.1221264_1221267del |
| G135Afs*23 | Frameshift Deletion | 19:g.1219350_1219363del |
| G163Afs*124 | Frameshift Deletion | 19:g.1220394del |
| G188Afs*99 | Frameshift Deletion | 19:g.1220469del |
| G196Afs*91 | Frameshift Deletion | 19:g.1220492del |
| G268Vfs*9 | Frameshift Deletion | 19:g.1221279_1221309del |
| G279Afs*8 | Frameshift Deletion | 19:g.1221312del |
| G279Rfs*6 | Frameshift Deletion | 19:g.1221311_1221317del |
| G279Vfs*8 | Frameshift Deletion | 19:g.1221313_1221314delinsT |
| I300Afs*12 | Frameshift Deletion | 19:g.1221983_1221999del |
| I300Gfs*2 | Frameshift Deletion | 19:g.1221983_1222003delinsGGAT |
| K311Nfs*4 | Frameshift Deletion | 19:g.1222996_1223003del |
| K44Tfs*2 | Frameshift Deletion | 19:g.1207042_1207057del |
| K64Rfs*32 | Frameshift Deletion | 19:g.1207102del |
| L117lfs*45 | Frameshift Deletion | 19:g.1218470_1218471del |
| L201Pfs*63 | Frameshift Deletion | 19:g.1220580_1220584del |
| L282Sfs*5 | Frameshift Deletion | 19:g.1221320del |
| LS0Rfs*12 | Frameshift Deletion | 19:g.1207061_1207067del |
| M136Nfs*24 | Frameshift Deletion | 19:g.1219351_1219358del |
| N181Tfs*106 | Frameshift Deletion | 19:g.1220444del |
| N226Rfs*39 | Frameshift Deletion | 19:g.1220659_1220660del |
| N90Tfs*6 | Frameshift Deletion | 19:g.1207176del |
| P217Rfs*70 | Frameshift Deletion | 19:g.1220629del |
| P221Afs*61 | Frameshift Deletion | 19:g.1220643_1220662delinsGCGC |
| P275Gfs*9 | Frameshift Deletion | 19:g.1221299_1221300del |
| P280Lfs*7 | Frameshift Deletion | 19:g.1221316_1221317delinsT |
| P281Gfs*3 | Frameshift Deletion | 19:g.1221318_1221321delinsGG |
| P281Rfs*6 | Frameshift Deletion | 19:g.1221314del |
| P314Lfs*22 | Frameshift Deletion | 19:g.1223003del |
| Q100Nfs*3 | Frameshift Deletion | 19:g.1218423del |
| Q100Pfs*51 | Frameshift Deletion | 19:g.1218422_1218456del |
| Q152Rfs*9 | Frameshift Deletion | 19:g.1219401del |
| R211Lfs*76 | Frameshift Deletion | 19:g.1220613_1220615delinsTT |
| R39Pfs*8 | Frameshift Deletion | 19:g.1207026_1207038del |
| R40Afs*ll | Frameshift Deletion | 19:g.1207029del |
| R42Lfs*9 | Frameshift Deletion | 19:g.1207036_1207038delinsTT |
| S216Rfs*43 | Frameshift Deletion | 19:g.1220627_1220646del |
| S283* | Frameshift Deletion | 19:g.1221321_1221322del |
| T249Gfs*37 | Frameshift Deletion | 19:g.1221222_1221226delinsG |
| V243Sfs*44 | Frameshift Deletion | 19:g.1220706del |
| W332Cfs*4 | Frameshift Deletion | 19:g.1223058del |
| Y36Sfs*125 | Frameshift Deletion | 19:g.1207019_1207023del |
| Y60* | Frameshift Deletion | 19:g.1207092del |
| Y60Sfs*4 | Frameshift Deletion | 19:g.1207091del |
| A218Gfs*48 | Frameshift Insertion | 19:g.1220628_1220629insC |
| A76Gfs*21 | Frameshift Insertion | 19:g.1207135_1207136insGG |
| D343Gfs*17 | Frameshift Insertion | 19:g.1223087_1223088insA |
| D53Gfs*110 | Frameshift Insertion | 19:g.1207064_1207065insG |
| E145* | Frameshift Insertion | 19:g.1219380_1219381insT |
| E265* | Frameshift Insertion | 19:g.1221266_1221267insT |
| E57Gfs*106 | Frameshift Insertion | 19:g.1207076_1207077insG |
| F255lfs*11 | Frameshift Insertion | 19:g.1221239_1221240insA |
| H202Pfs*64 | Frameshift Insertion | 19:g.12205851220587delinsTCCC |
| L282Afs*3 | Frameshift Insertion | 19:g.1221313_1221314insC |
| V197Rfs*69 | Frameshift Insertion | 19:g.1220491_1220492insG |
| V243Gfs*23 | Frameshift Insertion | 19:g.1220705_1220706insG |
| V66Sfs*98 | Frameshift Insertion | 19:g.1207108_1207109delinsAGCACC |
| C19orf26-STK11 | Fusion | |
| fusion | | |
| C19orf26-STK11 | Fusion | |
| fusion | | |
| CACNA1A-STK11 | Fusion | |
| fusion | | |
| MIDN-STKll fusion | Fusion | |
| REEP6-STK11 | Fusion | |
| fusion | | |
| STK11-intragenic | Fusion | |
| STK11-SBNO2 | Fusion | |
| fusion | | |
| A205T | Missense Mutation | 19:g.1220595G > A |
| D162_G163delinsYF | Missense Mutation | 19:g.1220391_1220395delinsTACTT |
| D176G | Missense Mutation | 19:g.1220434A > G |
| D194G | Missense Mutation | 19:g.1220488A > G |
| D194N | Missense Mutation | 19:g.1220487G > A |
| D194V | Missense Mutation | 19:g.1220488A > T |
| D194Y | Missense Mutation | 19:g.1220487G > T |
| E165K | Missense Mutation | 19:g.1220400G > A |
| E16SQ | Missense Mutation | 19:g.1220400G > C |
| E223K | Missense Mutation | 19:g.1220649G > A |
| E223V | Missense Mutation | 19:g.1220650A > T |
| F354L | Missense Mutation | 19:g.1223125C > G |
| G163R | Missense Mutation | 19:g.1220394G > C |
| G163S | Missense Mutation | 19:g.1220394G > A |
| G196R | Missense Mutation | 19:g.1220493G > C |
| G196V | Missense Mutation | 19:g.1220494G > T |
| G242R | Missense Mutation | 19:g.1220706G > A |
| G242V | Missense Mutation | 19:g.1220707_1220708delinsTT |
| G242W | Missense Mutation | 19:g.1220706G > T |
| G251C | Missense Mutation | 19:g.1221228G > T |
| G251V | Missense Mutation | 19:g.1221229G > T |
| H174D | Missense Mutation | 19:g.1220427C > G |
| H174L | Missense Mutation | 19:g.1220428A > T |
| H174R | Missense Mutation | 19:g.1220428A > G |
| K78N | Missense Mutation | 19:g.1207146G > T |
| N181S | Missense Mutation | 19:g.1220449A > G |
| N181T | Missense Mutation | 19:g.1220449A > C |
| P179L | Missense Mutation | 19:g.1220443C > T |
| P179Q | Missense Mutation | 19:g.1220443C > A |
| P179R | Missense Mutation | 19:g.1220443C > G |
| P221L | Missense Mutation | 19:g.1220644C > T |
| P281L | Missense Mutation | 19:g.1221319C > T |
| R304W | Missense Mutation | 19:g.1221995C > T |
| R409W | Missense Mutation | 19:g.1226569C > T |
| S216F | Missense Mutation | 19:g.1220629C > T |
| W239C | Missense Mutation | 19:g.1220699G > T |
| W239S | Missense Mutation | 19:g.1220698G > C |
| W308C | Missense Mutation | 19:g.1222987G > T |
| W308L | Missense Mutation | 19:g.1222986G > T |
| E120* | Nonsense Mutation | 19:g.1218483G > T |
| E120_E121delinsK* | Nonsense Mutation | 19:g.1218483_1218486delinsAAAT |
| E130* | Nonsense Mutation | 19:g.1219336G > T |
| E165* | Nonsense Mutation | 19:g.1220400G > T |
| E223* | Nonsense Mutation | 19:g.1220649G > T |
| E256* | Nonsense Mutation | 19:g.1221243G > T |
| E291* | Nonsense Mutation | 19:g.1221956G > T |
| E293* | Nonsense Mutation | 19:g.1221962G > T |
| E33* | Nonsense Mutation | 19:g.1207009G > T |
| E57* | Nonsense Mutation | 19:g.1207081G > T |
| E65* | Nonsense Mutation | 19:g.1207105G > T |
| E70* | Nonsense Mutation | 19:g.1207120G > T |
| K108* | Nonsense Mutation | 19:g.1218447A > T |
| K146* | Nonsense Mutation | 19:g.1219384A > T |
| K191* | Nonsense Mutation | 19:g.1220478A > T |
| K329* | Nonsense Mutation | 19:g.1223048A > T |
| K44* | Nonsense Mutation | 19:g.1207042A > T |
| K64* | Nonsense Mutation | 19:g.1207102A > T |
| K78* | Nonsense Mutation | 19:g.1207144A > T |
| K84* | Nonsense Mutation | 19:g.1207162A > T |
| K96* | Nonsense Mutation | 19:g.1207198A > T |
| K97* | Nonsense Mutation | 19:g.1207201A > T |
| L85* | Nonsense Mutation | 19:g.1207166T > A |
| Q100* | Nonsense Mutation | 19:g.1218423C > T |
| Q123* | Nonsense Mutation | 19:g.1218492C > T |
| Q159* | Nonsense Mutation | 19:g.1220382C> |
| Q170* | Nonsense Mutation | 19:g.1220415C > T |
| Q214* | Nonsense Mutation | 19:g.1220622C > T |
| Q220* | Nonsense Mutation | 19:g.1220640C > T |
| Q305* | Nonsense Mutation | 19:g.1221998C > T |
| Q37* | Nonsense Mutation | 19:g.1207021C > T |
| S240* | Nonsense Mutation | 19:g.1220701C > A |
| S69* | Nonsense Mutation | 19:g.1207118C > A |
| W239* | Nonsense Mutation | 19:g.1220699G > A |
| W308* | Nonsense Mutation | 19:g.1222987G > A |
| W332* | Nonsense Mutation | 19:g.1223059G > A |
| Y118* | Nonsense Mutation | 19:g.1218479C > G |
| Y131* | Nonsense Mutation | 19:g.1219341C > A |
| Y246* | Nonsense Mutation | 19:g.1221215C > G |
| Y253* | Nonsense Mutation | 19:g.1221236C > A |
| Y272* | Nonsense Mutation | 19:g.1221293C > A |
| Y36* | Nonsense Mutation | 19:g.1207020C > G |
| Y36_K48delins* | Nonsense Mutation | 19:g.1207019_1207054del |
| Y60* | Nonsense Mutation | 19:g.1207092C > A |
| Y60* | Nonsense Mutation | 19:g.1207090_1207091insA |
| Y60* | Nonsense Mutation | 19:g.1207092C > G |
| Y60* | Nonsense Mutation | 19:g.1207090_1207091insAG |
| X155_splice | Splice-Site | 19:g.1219413G > C |
| X288_splice | Splice-Site | 19:g.1221340G > A |
| X307_splice | Splice-Site | 19:g.1222983G > C |
| X307_splice | Splice-Site | 19:g.1222983G > T |
| X97_splice | Splice-Site | 19:g.1207201_1207203delinsGAT |
| X125_splice | Splice-Site | 19:g.1218500A > G |
| X125_splice | Splice-Site | 19:g.1219321A > T |
| X125_splice | Splice-Site | 19:g.1219322C > T |
| X148_splice | Splice-Site | 19:g.1219386_1219414del |
| X155_splice | Splice-Site | 19:g.1220370A > T |
| X155_splice | Splice-Site | 19:g.1220371G > T |
| X155_splice | Splice-Site | 19:g.1219413G > T |
| X155_splice | Splice-Site | 19:g.1220370A > G |
| X155_splice | Splice-Site | 19:g.1219412_1219422del |
| X155_splice | Splice-Site | 19:g.1220371_1220372delinsTT |
| X155_splice | Splice-Site | 19:g.1219414T > A |
| X155_splice | Splice-Site | 19:g.1220370A > C |
| X155_splice | Splice-Site | 19:g.1220201_1220383del |
| X171_splice | Splice-Site | 19:g.1220416_1220760del |
| X199_splice | Splice-Site | 19:g.1220504_1220544del |
| X199_splice | Splice-Site | 19:g.1220506T > C |
| X200_splice | Splice-Site | 19:g.1220578A > G |
| X200_splice | Splice-Site | 19:g.1220578A > T |
| X200_splice | Splice-Site | 19:g.1220574_1220607del |
| X245_splice | Splice-Site | 19:g.1220717G > T |
| X245_splice | Splice-Site | 19:g.1220717G > A |
| X245_splice | Splice-Site | 19:g.1220718T > G |
| X245_splice | Splice-Site | 19:g.1220717G > C |
| X285_splice | Splice-Site | 19:g.1221332_1221361del |
| X288_splice | Splice-Site | 19:g.1221946A > C |
| X288_splice | Splice-Site | 19:g.1221947G > A |
| X288_splice | Splice-Site | 19:g.1221947G > T |
| X307_splice | Splice-Site | 19:g.1222006G > T |
| X307_splice | Splice-Site | 19:g.1222983G > A |
| X307_splice | Splice-Site | 19:g.1222982A > G |
| X307_splice | Splice-Site | 19:g.1222982A > T |
| X307_splice | Splice-Site | 19:g.1222611_1223004del |
| X97_splice | Splice-Site | 19:g.1218415G > T |
| X97_splice | Splice-Site | 19:g.1207203G > A |
| X97_splice | Splice-Site | 19:g.1207204T > A |
| M1? | Translation Start Site | 19:g.1206915G > A |

One of skill in the art would also appreciate that STK11 mutations may co-occur with other mutations. Mutations in STK11 co-occur frequently with KRAS mutations. (Koivunen, J. et al. Br J Cancer 2008, 99, 245-252). STK11 somatic mutations also co-occur frequently with KEAP1 mutations (See Papillon-Cavanagh S. et al. ESMO Open, 2020, 5, E000706). Some authors report that the presence of STK11 and KEAP1 mutations have a bigger impact on immunotherapy resistance in patients with KRAS mutations than in patients with wild-type KRAS (See Ricciuti B. et al. Journal of Thoracic Oncology 2021, 17, 400-410).

In some embodiments provided herein, the cancer is identified as having modified STK11 activity or expression and as having modified KRAS activity or expression. In some embodiment, the modified KRAS activity or expression is the presence of a mutant KRAS. In some embodiment, the mutant KRAS is selected from KRAS^{G12C}, KRAS^{G12D}, KRAS^{G12V}, KRAS^{G12A}, KRAS^{G12S}, KRAS^{G12R}, KRAS^{G13C}, KRAS^{G13D}, KRAS^{G13S}, KRAS^{Q61H} and KRAS^{Q61K}. In some embodiments, the mutant KRAS is selected from KRAS^{G12C}, KRAS^{G12D} and KRAS^{G12V}. In some embodiments provided herein, the cancer is identified as having modified STK11 activity or expression and as having wildtype KRAS activity or expression. In some embodiments provided herein, the cancer is identified as having modified STK11 activity or expression and as having modified KRAS (e.g., KRAS^{G12C}, KRAS^{G12D}, KRAS^{G12V}) activity or expression or wildtype KRAS activity or expression. In some embodiments, the cancer is further identified as having modified KEAP1 activity or expression (e.g., KEAP1 mutations).

One of skill in the art would also appreciate that STK11 mutations may frequently occur in certain diseases (e.g., cancer).

### Histone Deacetylase Inhibitors

In some embodiments, the method of treating a subject having, or at risk of developing, a cancer described therein comprises administering to the subject a histone deacetylase (HDAC) inhibitor. Unless otherwise specified, references to HDAC inhibitors in the methods and uses described herein refer to any of the HDAC inhibitor classes and HDAC inhibitor compounds described herein (e.g., in this section).

Histone deacetylase (HDAC) inhibitors are generally a class of therapeutic drugs that inhibit histone deacetylases. The 18 isoforms of histone deacetylases have been identified and classified into four classes: Class I, Class II, Class III, and Class IV. Class II HDACs are further grouped into Class IIa and IIb. The four classes distinguish the 18 identified isoforms of HDACs into two primary families: HDACs 1-11 that are zinc dependent metalloenzymes and SIRTs 1-7. Class I HDACs include HDAC1. HDAC2, HDAC3, and HDAC8. Class II HDACs include Class Ila and Class IIb. Class IIa HDACs include HDAC4. HDAC 5, HDAC7, and HDAC9. Class IIb HDACs include HDAC6 and HDAC10. Class III HDACs, also known as sirtuins (SIRTs) include SIRT1, SIRT2, SIRT3, SIRT4, SIRT5, SIRT6, and SIRT7. And Class IV HDACs include HDAC11. A review of HDACs including compositions and sequence characteristics is described in Li, G. et al Frontiers in Cell and Developmental Biology (2020) 8:576946 (doi: 10.3389/fcell.2020.576946), which is hereby incorporated by reference in its entirety.

HDAC inhibitors can either be pan-HDAC inhibitors, which generally inhibit most or all HDAC isoforms, or selective HDAC inhibitors, which generally inhibit one or more HDAC isoforms selectively over other HDAC isoforms. Many HDAC inhibitors have been described in the art.

For example, a review of HDAC inhibitors over thirty years of development is described in Ho, T. C. S., et al. Journal of Medicinal Chemistry (2020) 63 (21): 12460-12484 (doi: 10.1021/acs.jmedchem.0c00830), which is hereby incorporated by reference in its entirety. A review of developments of HDAC inhibitors is described in Bondarev, A., et al. British Journal of Clinical Pharmacology (2021) 87:4577-4597 (doi: 10.1111/bcp.14889), which is hereby incorporated by reference in its entirety. A review of hybridized multi-targeting HDAC inhibitors is described in Bass, A. K. A., et al. European Journal of Medicinal Chemistry (2021) 209:112904 (doi: 10.1016/j.ejmech.2020.112904), which is hereby incorporated by reference in its entirety. One of skill in the art would appreciate that many HDAC inhibitors have been described in the art via patent publications, posters, conferences, and journals. Some of these HDAC inhibitors are described as pan-HDAC inhibitors. Some of these HDAC inhibitors are described as selective HDAC inhibitors, inhibiting a specific class of HDACs (e.g., Class I, Class Ila, Class IIb, Class III, or Class IV) or inhibiting specific isoforms of HDAC (e.g., HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, and/or SIRT7).

Examples of selective HDAC inhibitors have been described in the art. For example, HDAC inhibitors are described in international patent publications WO/2010/014611 and WO/2010/144371, each of which is hereby incorporated by reference in their entirety.

“Selective” as used herein and in reference to HDAC inhibitors refer to a compound's HDAC inhibitory properties that preferentially inhibit one or more HDAC isoforms, including preferentially inhibiting one or more HDAC isoforms in a specific biological complex. A selective HDAC inhibitor inhibits the target HDACs (including, e.g., HDAC isoforms that are part of specific complexes such as CoREST) at a lower concentration than the concentration at which it inhibits the non-target HDACs (including e.g., the same HDAC isoforms as part of different complexes). Thus, a selective HDAC inhibitor is more potent (has a lower IC₅₀) against the target HDACs than against the non-target HDACs. In one embodiment, a selective HDAC inhibitor is at least 3 times more potent against the target HDACs than against non-target HDACs (i.e., has an IC₅₀ for the target HDACs that is at least 3 times lower than the IC₅₀ for the non-target HDACs). In one embodiment, a selective HDAC inhibitor is at least 5 times more potent against the target HDACs than against non-target HDACs (i.e., has an IC₅₀ for the target HDACs that is at least 5 times lower than the IC₅₀ for the non-target HDACs). In one embodiment, a selective HDAC inhibitor is at least 10 times more potent against the target HDACs than against non-target HDACs (i.e., has an IC₅₀ for the target HDACs that is at least 10 times lower than the IC₅₀ for the non-target HDACs). In one embodiment, a selective HDAC inhibitor is at least 30 times more potent against the target HDACs than against non-target HDACs (i.e., has an IC₅₀ for the target HDACs that is at least 30 times lower than the IC₅₀ for the non-target HDACs). In one embodiment, a selective HDAC inhibitor is at least 50 times more potent against the target HDACs than against non-target HDACs (i.e., has an IC₅₀ for the target HDACs that is at least 50 times lower than the IC₅₀ for the non-target HDACs). In one embodiment, a selective HDAC inhibitor is at least 100 times more potent against the target HDACs than against non-target HDACs (i.e., has an IC₅₀ for the target HDACs that is at least 100 times lower than the IC₅₀ for the non-target HDACs). In one embodiment, a selective HDAC inhibitor is at least 500 times more potent against the target HDACs than against non-target HDACs (i.e., has an IC₅₀ for the target HDACs that is at least 500 times lower than the IC₅₀ for the non-target HDACs). In one embodiment, a selective HDAC inhibitor is at least 1000 times more potent against the target HDACs than against non-target HDAC's (i.e., has an IC₅₀ for the target HDACs that is at least 1000 times lower than the IC₅₀ for the non-target HDACs).

For example, HDAC1,2-selective inhibitors are described in international patent publications WO/2016/094824, WO/2016/109549, WO/2018/098296. WO/2019/012172, WO/2020/068950, and WO/2020/076951, each of which is hereby incorporated by reference in their entirety.

Certain biological complexes include certain classes or isoforms of HDACs. For example, at least four biological complexes comprise various Class I HDAC isoforms as well as other subunits. The HDAC-co-repressor of repressor element-1 silencing transcription factor (CoREST) complex comprises HDAC1 and HDAC2, as well as other subunits such as LSD1 and RCOR1. The nucleosome remodeling and deacetylase (NuRD) complex also comprises HDAC1 and HDAC2, as well as other subunits such as MTA3 and RBBP7. The Sin3-HDAC (Sin3) complex has also been described as comprising HDAC1 and HDAC2 as well as other subunits Sin3 and RBBP7. The NCoR complex has been described as comprising HDAC3 as well as other subunits NCOR, HSPA, and TBL1.

HDAC inhibitors may selectively inhibit certain isoforms of certain biological complexes over the same isoforms in different complexes. For examples, CoREST Complex-Selective HDAC inhibitors are described in Fuller. N. O., et al. (2019) CS Chem. Neurosci. 10 (3): 1729-1743 (10.1021/acschemneuro.8b00620), which is hereby incorporated by reference in its entirety. Fuller et al. describes certain efforts where HDACs in the benzamide chemical class (CI-994 and BML-210) demonstrated selectivity for CoREST. NuRD, and NCoR, but not the Sin3 complex. Fuller et al. further describes compounds that selectively target the HDACs (e.g., HDAC 1 and HDAC2) in the CoREST complex.

The structures of the CoREST Complex-Selective HDAC inhibitors described in Fuller are shown below:

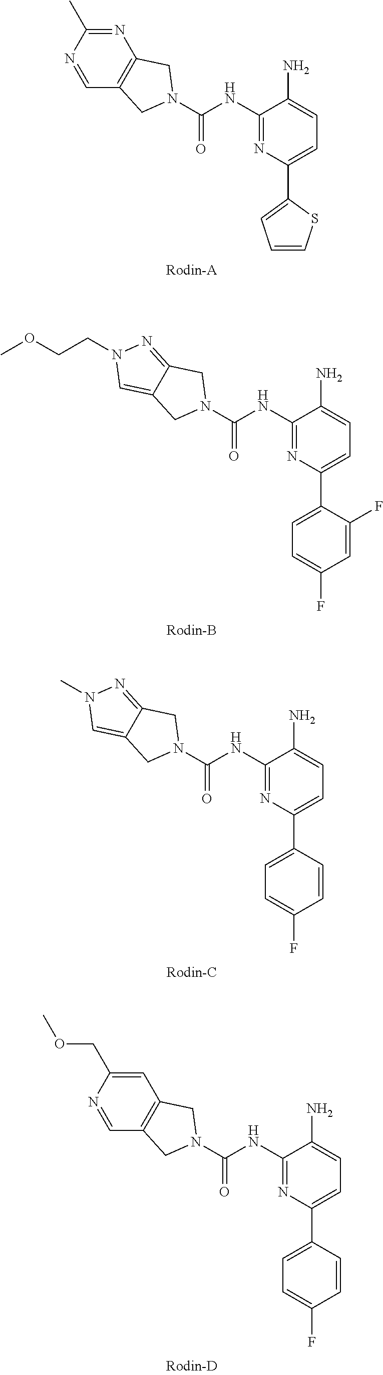 [SMILES: Cc1ncc2c(n1)CN(C(=O)Nc1nc(-c3cccs3)ccc1N)C2 ; COCCn1cc2c(n1)CN(C(=O)Nc1nc(-c3ccc(F)cc3F)ccc1N)C2 ; Cn1cc2c(n1)CN(C(=O)Nc1nc(-c3ccc(F)cc3)ccc1N)C2 ; COCc1cc2c(cn1)CN(C(=O)Nc1nc(-c3ccc(F)cc3)ccc1N)C2]

Reports describing other compounds that selectively target the HDACs (e.g., HDAC 1 and HDAC2) in the CoREST complex have identified a compound “RDN-929” have also published.

In some embodiments, the histone deacetylase inhibitor is an HDAC inhibitor that selectively inhibits HDAC1 over HDAC3. In some embodiments, the histone deacetylase inhibitor is an HDAC inhibitor that selectively inhibits HDAC1 and HDAC2 over HDAC3. In some embodiments, the histone deacetylase inhibitor is an HDAC inhibitor that selectively inhibits HDAC1 over HDAC8. In some embodiments, the histone deacetylase inhibitor is an HDAC inhibitor that selectively inhibits HDAC1 and HDAC2 over HDAC8. In some embodiments, the histone deacetylase inhibitor is an HDAC inhibitor that selectively inhibits HDAC1 over HDAC3 and HDAC8. In some embodiments, the histone deacetylase inhibitor is an HDAC inhibitor that selectively inhibits HDAC1 and HDAC2 over HDAC3 and HDAC8.

In some embodiments, the histone deacetylase inhibitor is an HDAC Class I-selective inhibitor. In some embodiments, the histone deacetylase inhibitor is an HDAC Class I inhibitor that selectively inhibits HDAC1 over HDAC3. In some embodiments, the histone deacetylase inhibitor is an HDAC Class I inhibitor that selectively inhibits HDAC1 and HDAC2 over HDAC3. In some embodiments, the histone deacetylase inhibitor is an HDAC Class I inhibitor that selectively inhibits HDAC1 over HDAC8. In some embodiments, the histone deacetylase inhibitor is an HDAC Class I inhibitor that selectively inhibits HDAC1 and HDAC2 over HDAC8. In some embodiments, the histone deacetylase inhibitor is an HDAC Class I inhibitor that selectively inhibits HDAC1 over HDAC3 and HDAC8. In some embodiments, the histone deacetylase inhibitor is an HDAC Class I inhibitor that selectively inhibits HDAC1 and HDAC2 over HDAC3 and HDAC8. In some embodiments, the histone deacetylase inhibitor is an HDAC Class I inhibitor that selectively inhibits HDAC1 and HDAC2 over all other HDAC isoforms.

In some embodiments, the method of treating a subject having, or at risk of developing, a cancer, the method comprises administering to the subject a selective HDAC1 inhibitor.

In some embodiments, the histone deacetylase inhibitor is an HDAC1-selective inhibitor. In some embodiments, the histone deacetylase inhibitor is an HDAC1,2-selective inhibitor. In some embodiments, the histone deacetylase inhibitor is an HDAC1-selective inhibitor that selectively inhibits HDAC 1 over HDAC3. In some embodiments, the histone deacetylase inhibitor is an HDAC1,2-selective inhibitor that selectively inhibits HDAC 1,2 over HDAC3. In some embodiments, the histone deacetylase inhibitor is an HDAC1-selective inhibitor that selectively inhibits HDAC 1 over HDAC8. In some embodiments, the histone deacetylase inhibitor is an HDAC1,2-selective inhibitor that selectively inhibits HDAC 1,2 over HDAC8. In some embodiments, the histone deacetylase inhibitor is an HDAC1-selective inhibitor that selectively inhibits HDAC 1 over HDAC3 and HDAC8. In some embodiments, the histone deacetylase inhibitor is an HDAC1,2-selective inhibitor that selectively inhibits HDAC 1,2 over HDAC3 and HDAC8.

In some embodiments, the method of treating a subject having, or at risk of developing, a cancer, the method comprises administering to the subject a CoREST-selective deacetylase inhibitor.

In some embodiments, the histone deacetylase inhibitor is a CoREST-selective deacetylase inhibitor. In some embodiments, the histone deacetylase inhibitor is a CoREST-selective deacetylase inhibitor that inhibits HDAC1. In some embodiments, the histone deacetylase inhibitor is a CoREST-selective deacetylase inhibitor that inhibits HDAC1 and HDAC2.

In some embodiments, the selective HDAC1,2 inhibitor has reduced cytotoxicity and an improved therapeutic index over less-selective HDAC inhibitors. In some embodiments, the selective HDAC1,2 inhibitor has reduced cytotoxicity against erythroid cells and or myeloid cells.

In some embodiments of any of the methods and uses described herein, the histone deacetylase (HDAC) inhibitor is (R)—N-(4-amino-4′-fluoro-[1,1′-biphenyl]-3-yl)-4-(S-methylsulfonimidoyl)benzamide, having Formula (I) (Compound I) also known as TNG260.

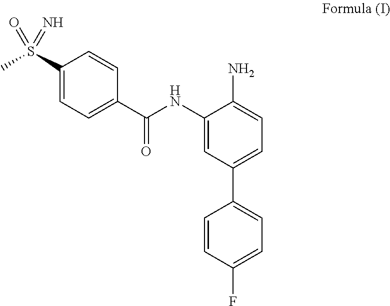 [SMILES: C[S@@](=N)(=O)c1ccc(C(=O)Nc2cc(-c3ccc(F)cc3)ccc2N)cc1]

or a pharmaceutically acceptable salt thereof.

### Pharmaceutical Compositions and Administration

Generally, the histone deacetylase (HDAC) inhibitors are administered in an effective amount (e.g., a therapeutically effective amount). The amount of the compound described herein (e.g., HDAC inhibitor) actually administered will typically be determined by a physician, in the light of the relevant circumstances, including the condition to be treated, the chosen route of administration, the actual HDAC inhibitor administered, the age, weight, and response of the individual patient, the severity of the patient's symptoms, and the like.

When employed as pharmaceuticals, the compounds described herein (e.g., HDAC inhibitor) are typically administered in the form of a pharmaceutical composition. Such compositions can be prepared in a manner well known in the pharmaceutical art and comprise at least one active compound.

In some embodiments, the compound described herein (e.g., HDAC inhibitor) is administered as a pharmaceutical composition comprising an effective amount of an HDAC inhibitor as described herein, and a pharmaceutically acceptable carrier. In some embodiments, with respect to the pharmaceutical composition, the carrier is a parenteral carrier, oral or topical carrier. The term “pharmaceutically acceptable carrier” refers to a carrier, adjuvant, or vehicle that may be administered to a patient, together with a compound provided herewith, and which does not destroy the pharmacological activity thereof and is nontoxic when administered in doses sufficient to deliver a therapeutic amount of the compound. Pharmaceutically acceptable carriers that may be used in the pharmaceutical compositions provided herewith include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices. serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose based substances, polyethylene glycol, sodium carboxymethylcellulose, polyacrylates, waxes, polyethylene polyoxypropylene block polymers, polyethylene glycol and wool fat. Cyclodextrins such as α-, β-, and γ-cyclodextrin, or chemically modified derivatives such as hydroxyalkylcyclodextrins, including 2 and 3 hydroxypropyl-β-cyclodextrins, or other solubilized derivatives may also be advantageously used to enhance delivery of the compounds (e.g., HDAC inhibitor) described herein.

The pharmaceutical compositions provided herewith may be administered orally, parenterally. by inhalation spray, topically, rectally, nasally, buccally, vaginally or via an implanted reservoir, preferably by oral administration or administration by injection. The pharmaceutical compositions provided herewith may contain any conventional nontoxic pharmaceutically acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature; a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like. The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The HDAC inhibitors provided herein can also be administered by a transdermal device. Accordingly, transdermal administration can be accomplished using a patch either of the reservoir or porous membrane type, or of a solid matrix variety.

The pharmaceutical compositions provided herewith may also be administered in the form of suppositories for rectal administration. These compositions can be prepared by mixing a compound provided herewith with a suitable nonirritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions provided herewith may be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The above-described components for orally administrable, injectable or topically administrable, rectally administrable and nasally administrable compositions are merely representative. Other materials as well as processing techniques and the like are set forth in Part 8 of Remington's Pharmaceutical Sciences, 17th edition, 1985, Mack Publishing Company, Easton, Pennsylvania, which is hereby incorporated in its entirety.

The compounds (e.g., HDAC inhibitors) described herein can also be administered in sustained release forms or from sustained release drug delivery systems. A description of representative sustained release materials can be found in Remington's Pharmaceutical Sciences.

When the compositions provided herewith comprise a combination of a compound (e.g., HDAC inhibitor) described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds provided herewith. Alternatively, those agents may be part of a single dosage form, mixed together with the compounds provided herewith in a single composition.

The compounds (e.g., HDAC inhibitors) described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or composition to achieve the desired or stated effect. Typically, the pharmaceutical compositions provided herewith will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound (e.g., HDAC inhibitor), composition or combination provided herewith may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long term basis upon any recurrence of disease symptoms.

### “Effective Amount”

In general, the “effective amount” of a compound (e.g., HDAC inhibitor) refers to an amount sufficient to elicit the desired biological response e.g., to treat a disease or disorder described herein. As will be appreciated by those of ordinary skill in this art, the effective amount of a compound of the disclosure may vary depending on such factors as the desired biological endpoint, the pharmacokinetics of the compound, the disease being treated, the mode of administration, and the age, health, and condition of the subject. An effective amount encompasses therapeutic and prophylactic treatment (i.e., encompasses a “therapeutically effective amount” and a “prophylactically effective amount”).

As used herein, and unless otherwise specified, a “therapeutically effective amount” of a compound is an amount sufficient to provide a therapeutic benefit in the therapeutic treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the therapeutic treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

### Methods of Treatment

Provided herein are methods of treating a subject having, or at risk of developing, a disease or disorder, the method comprising administering to the subject an effective amount of a histone deacetylase inhibitor.

In some embodiments, provided is a method of treating a subject having, or at risk of developing, a disease or disorder, the method comprising administering to the subject an effective amount of a histone deacetylase inhibitor, wherein the cancer is identified as having modified STK11 activity or expression.

In some embodiments, provided is a method of treating a subject having, or at risk of developing, a cancer, the method comprising administering to the subject an effective amount of a histone deacetylase inhibitor, wherein the cancer is identified as having modified STK11 activity or expression.

In some embodiments, the method involves selecting a patient for treatment using one of the Patient Selection methods described herein, prior to administering to the patient the histone deacetylase inhibitor and optionally one or more additional therapeutic agents.

In some embodiments, provided is a method of treating a subject having, or at risk of developing, a cancer, the method comprising administering to the subject an effective amount of a histone deacetylase inhibitor in combination with a second therapeutic agent, wherein the cancer is identified as having modified STK11 activity or expression. In some embodiments, provided is a method of treating a subject having, or at risk of developing, a cancer, the method comprising administering to the subject an effective amount of a histone deacetylase inhibitor in combination with a second therapeutic agent, wherein the cancer is identified as having modified STK11 activity or expression and the cancer is identified as having wildtype KRAS activity or expression. In some embodiments, provided is a method of treating a subject having, or at risk of developing, a cancer, the method comprising administering to the subject an effective amount of a histone deacetylase inhibitor in combination with a second therapeutic agent, wherein the cancer is identified as having modified STK11 activity or expression and the cancer is identified as having modified KRAS (e.g., KRAS^{G12C}, KRAS^{G12D}, KRAS^{G12V}) activity or expression. In some embodiments, provided is a method of treating a subject having, or at risk of developing, a cancer, the method comprising administering to the subject an effective amount of a histone deacetylase inhibitor in combination with a second therapeutic agent, wherein the cancer is identified as having modified STK11 activity or expression and the cancer is identified as having modified KRAS (e.g., KRAS^{G12C}, KRAS^{G12D}, KRAS^{G12V}) activity or expression or wildtype KRAS activity or expression.

In some embodiments, provided is a method of treating a subject having, or at risk of developing, a cancer, the method comprising administering to the subject an effective amount of a histone deacetylase inhibitor in combination with an immune checkpoint modulator, wherein the cancer is identified as having modified STK11 activity or expression. In some embodiments, provided is a method of treating a subject having, or at risk of developing, a cancer, the method comprising administering to the subject an effective amount of a histone deacetylase inhibitor in combination with an anti-PD1 agent or an anti-PD-L1 agent, wherein the cancer is identified as having modified STK11 activity or expression. In some embodiments, provided is a method of treating a subject having, or at risk of developing, a cancer, the method comprising administering to the subject an effective amount of a histone deacetylase inhibitor in combination with an anti-PD1 agent or an anti-PD-L1 agent, wherein the cancer is identified as having modified STK11 activity or expression, wherein the anti-PD1 agent or the anti-PD-L1 agent is selected from the group consisting of nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab. BMS936559; durvalumab; avelumab; envafolimab; cosibelimab; sugemalimab. AUNP-12; atezolizumab and CA-170. In some embodiments, provided is a method of treating a subject having, or at risk of developing, a cancer, the method comprising administering to the subject an effective amount of a histone deacetylase inhibitor in combination with an anti-PD1 therapy or an anti-PD-L1 therapy, wherein the cancer is identified as having modified STK11 activity or expression.

In some embodiments, provided is a method of treating a subject having, or at risk of developing, a cancer, the method comprising administering to the subject an effective amount of a selective histone deacetylase inhibitor in combination with a second therapeutic agent, wherein the cancer is identified as having modified STK11 activity or expression. In some embodiments, provided is a method of treating a subject having, or at risk of developing, a cancer, the method comprising administering to the subject an effective amount of a selective histone deacetylase inhibitor in combination with an immune checkpoint modulator, wherein the cancer is identified as having modified STK11 activity or expression. In some embodiments, provided is a method of treating a subject having, or at risk of developing, a cancer, the method comprising administering to the subject an effective amount of a selective histone deacetylase inhibitor in combination with an anti-PD1 agent or an anti-PD-L1 agent, wherein the cancer is identified as having modified STK11 activity or expression. In some embodiments, provided is a method of treating a subject having, or at risk of developing, a cancer, the method comprising administering to the subject an effective amount of a selective histone deacetylase inhibitor in combination with an anti-PD1 agent or an anti-PD-L1 agent, wherein the cancer is identified as having modified STK11 activity or expression, wherein the anti-PD1 agent or the anti-PD-L1 agent is selected from the group consisting of nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab, BMS936559; durvalumab; avelumab; envafolimab; cosibelimab; sugemalimab, AUNP-12; atezolizumab and CA-170. In some embodiments, provided is a method of treating a subject having, or at risk of developing, a cancer, the method comprising administering to the subject an effective amount of a selective histone deacetylase inhibitor in combination with an anti-PD1 therapy or an anti-PD-L1 therapy, wherein the cancer is identified as having modified STK11 activity or expression.

In some embodiments, provided is a method of treating a subject having, or at risk of developing, a cancer, the method comprising administering to the subject an effective amount of an HDAC Class I-selective histone deacetylase inhibitor in combination with a second therapeutic agent, wherein the cancer is identified as having modified STK11 activity or expression. In some embodiments, provided is a method of treating a subject having, or at risk of developing, a cancer, the method comprising administering to the subject an effective amount of an HDAC Class I-selective inhibitor in combination with an immune checkpoint modulator, wherein the cancer is identified as having modified STK11 activity or expression. In some embodiments, provided is a method of treating a subject having, or at risk of developing, a cancer, the method comprising administering to the subject an effective amount of an HDAC Class I-selective inhibitor in combination with an anti-PD1 agent or an anti-PD-L1 agent, wherein the cancer is identified as having modified STK11 activity or expression. In some embodiments, provided is a method of treating a subject having, or at risk of developing, a cancer, the method comprising administering to the subject an effective amount of an HDAC Class I-selective inhibitor in combination with an anti-PD1 agent or an anti-PD-L1 agent, wherein the cancer is identified as having modified STK11 activity or expression, wherein the anti-PD1 agent or the anti-PD-L1 agent is selected from the group consisting of nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab, BMS936559; durvalumab; avelumab; envafolimab; cosibelimab; sugemalimab, AUNP-12; atezolizumab and CA-170. In some embodiments. provided is a method of treating a subject having, or at risk of developing, a cancer, the method comprising administering to the subject an effective amount of an HDAC Class I-selective inhibitor in combination with an anti-PD1 therapy or an anti-PD-L1 therapy, wherein the cancer is identified as having modified STK11 activity or expression.

In some embodiments, provided is a method of treating a subject having, or at risk of developing, a cancer, the method comprising administering to the subject an effective amount of an HDAC1-selective histone deacetylase inhibitor in combination with a second therapeutic agent, wherein the cancer is identified as having modified STK11 activity or expression. In some embodiments, provided is a method of treating a subject having, or at risk of developing, a cancer, the method comprising administering to the subject an effective amount of an HDAC1-selective inhibitor in combination with an immune checkpoint modulator, wherein the cancer is identified as having modified STK11 activity or expression. In some embodiments, provided is a method of treating a subject having, or at risk of developing, a cancer, the method comprising administering to the subject an effective amount of an HDAC1-selective inhibitor in combination with an anti-PD1 agent or an anti-PD-L1 agent, wherein the cancer is identified as having modified STK11 activity or expression. In some embodiments, provided is a method of treating a subject having, or at risk of developing, a cancer, the method comprising administering to the subject an effective amount of an HDAC1-selective inhibitor in combination with an anti-PD1 agent or an anti-PD-L1 agent, wherein the cancer is identified as having modified STK11 activity or expression, wherein the anti-PD1 agent or the anti-PD-L1 agent is selected from the group consisting of nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab. BMS936559; durvalumab; avelumab; envafolimab; cosibelimab; sugemalimab, AUNP-12; atezolizumab and CA-170. In some embodiments, provided is a method of treating a subject having, or at risk of developing, a cancer, the method comprising administering to the subject an effective amount of an HDAC1-selective inhibitor in combination with an anti-PD1 therapy or an anti-PD-L1 therapy, wherein the cancer is identified as having modified STK11 activity or expression.

In some embodiments, provided is a method of treating a subject having, or at risk of developing, a cancer, the method comprising administering to the subject an effective amount of an HDAC1,2-selective histone deacetylase inhibitor in combination with a second therapeutic agent, wherein the cancer is identified as having modified STK11 activity or expression. In some embodiments, provided is a method of treating a subject having, or at risk of developing, a cancer, the method comprising administering to the subject an effective amount of an HDAC1,2-selective inhibitor in combination with an immune checkpoint modulator, wherein the cancer is identified as having modified STK11 activity or expression. In some embodiments, provided is a method of treating a subject having, or at risk of developing, a cancer, the method comprising administering to the subject an effective amount of an HDAC1,2-selective inhibitor in combination with an anti-PD1 agent or an anti-PD-L1 agent, wherein the cancer is identified as having modified STK11 activity or expression. In some embodiments, provided is a method of treating a subject having, or at risk of developing, a cancer, the method comprising administering to the subject an effective amount of an HDAC1,2-selective inhibitor in combination with an anti-PD1 agent or an anti-PD-L1 agent, wherein the cancer is identified as having modified STK11 activity or expression, wherein the anti-PD1 agent or the anti-PD-L1 agent is selected from the group consisting of nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab, BMS936559; durvalumab; avelumab; envafolimab; cosibelimab; sugemalimab, AUNP-12; atezolizumab, and CA-170. In some embodiments, provided is a method of treating a subject having, or at risk of developing, a cancer, the method comprising administering to the subject an effective amount of an HDAC1,2-selective inhibitor in combination with an anti-PD1 therapy or an anti-PD-L1 therapy, wherein the cancer is identified as having modified STK11 activity or expression.

In some embodiments, provided is a method of treating a subject having, or at risk of developing, a cancer, the method comprising administering to the subject an effective amount of a CoREST-selective deacetylase inhibitor in combination with a second therapeutic agent, wherein the cancer is identified as having modified STK11 activity or expression. In some embodiments, provided is a method of treating a subject having, or at risk of developing, a cancer, the method comprising administering to the subject an effective amount of a CoREST-selective deacetylase inhibitor in combination with an immune checkpoint modulator, wherein the cancer is identified as having modified STK11 activity or expression. In some embodiments, provided is a method of treating a subject having, or at risk of developing, a cancer, the method comprising administering to the subject an effective amount of a CoREST-selective deacetylase inhibitor in combination with an anti-PD1 agent or an anti-PD-L1 agent, wherein the cancer is identified as having modified STK11 activity or expression. In some embodiments, provided is a method of treating a subject having, or at risk of developing, a cancer, the method comprising administering to the subject an effective amount of a CoREST-selective deacetylase inhibitor in combination with an anti-PD1 agent or an anti-PD-L1 agent, wherein the cancer is identified as having modified STK11 activity or expression, wherein the anti-PD1 agent or the anti-PD-L1 agent is selected from the group consisting of nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab, BMS936559; durvalumab; avelumab; envafolimab; cosibelimab; sugemalimab, AUNP-12; atezolizumab, and CA-170. In some embodiments, provided is a method of treating a subject having, or at risk of developing, a cancer, the method comprising administering to the subject an effective amount of a CoREST-selective deacetylase inhibitor in combination with an anti-PD1 therapy or an anti-PD-L1 therapy, wherein the cancer is identified as having modified STK11 activity or expression.

In some embodiments, provided is a method of treating a subject having, or at risk of developing, a cancer, the method comprising administering to the subject an effective amount of a histone deacetylase inhibitor, wherein the cancer is identified as having modified STK11 activity or expression and the cancer is identified as resistant to anti-PD1 therapy or anti-PD-L1 therapy. In some embodiments, provided is a method of treating a subject having, or at risk of developing, a cancer, the method comprising administering to the subject an effective amount of a histone deacetylase inhibitor, wherein the cancer is identified as having modified STK11 activity or expression and the cancer is identified as having intrinsic resistance to anti-PD1 therapy or anti-PD-L1 therapy. In some embodiments, provided is a method of treating a subject having, or at risk of developing, a cancer, the method comprising administering to the subject an effective amount of a histone deacetylase inhibitor, wherein the cancer is identified as having modified STK11 activity or expression and the cancer is identified as having acquired resistance to anti-PD1 therapy or anti-PD-L1 therapy.

In some embodiments, provided is a method of treating a subject having, or at risk of developing, a lung cancer, the method comprising administering to the subject an effective amount of a histone deacetylase inhibitor in combination with a second therapeutic agent, wherein the lung cancer is identified as having modified STK11 activity or expression. In some embodiments, provided is a method of treating a subject having, or at risk of developing, a lung cancer, the method comprising administering to the subject an effective amount of a histone deacetylase inhibitor in combination with a second therapeutic agent, wherein the lung cancer is identified as having modified STK11 activity or expression and the lung cancer is identified as having wildtype KRAS activity or expression. In some embodiments, provided is a method of treating a subject having, or at risk of developing, a lung cancer, the method comprising administering to the subject an effective amount of a histone deacetylase inhibitor in combination with a second therapeutic agent, wherein the lung cancer is identified as having modified STK11 activity or expression and the lung cancer is identified as having modified KRAS (e.g., KRAS^{G12C}, KRAS^{G12D}, KRAS^{G12V}) activity or expression. In some embodiments, provided is a method of treating a subject having, or at risk of developing, a lung cancer, the method comprising administering to the subject an effective amount of a histone deacetylase inhibitor in combination with a second therapeutic agent, wherein the lung cancer is identified as having modified STK11 activity or expression and the lung cancer is identified as having modified KRAS (e.g., KRAS^{G12C}, KRAS^{G12D}, KRAS^{G12V}) activity or expression or wildtype KRAS activity or expression. In some embodiments, provided is a method of treating a subject having, or at risk of developing, a lung cancer, the method comprising administering to the subject an effective amount of a histone deacetylase inhibitor in combination with an immune checkpoint modulator, wherein the lung cancer is identified as having modified STK11 activity or expression. In some embodiments, provided is a method of treating a subject having, or at risk of developing, a lung cancer, the method comprising administering to the subject an effective amount of a histone deacetylase inhibitor in combination with an anti-PD1 agent or an anti-PD-L1 agent, wherein the lung cancer is identified as having modified STK11 activity or expression. In some embodiments, provided is a method of treating a subject having, or at risk of developing, a lung cancer, the method comprising administering to the subject an effective amount of a histone deacetylase inhibitor in combination with an anti-PD1 agent or an anti-PD-L1 agent, wherein the lung cancer is identified as having modified STK11 activity or expression, wherein the anti-PD1 agent or the anti-PD-L1 agent is selected from the group consisting of nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab, BMS936559; durvalumab; avelumab; envafolimab; cosibelimab; sugemalimab, AUNP-12; atezolizumab and CA-170. In some embodiments, provided is a method of treating a subject having, or at risk of developing, a lung cancer, the method comprising administering to the subject an effective amount of a histone deacetylase inhibitor in combination with an anti-PD1 therapy or an anti-PD-L1 therapy, wherein the lung cancer is identified as having modified STK11 activity or expression.

In some embodiments, provided is a method of treating a subject having, or at risk of developing, a lung cancer, the method comprising administering to the subject an effective amount of a histone deacetylase inhibitor, wherein the lung cancer is identified as having modified STK11 activity or expression and the lung cancer is identified as resistant to anti-PD1 therapy or anti-PD-L1 therapy. In some embodiments, provided is a method of treating a subject having, or at risk of developing, a lung cancer, the method comprising administering to the subject an effective amount of a histone deacetylase inhibitor, wherein the lung cancer is identified as having modified STK11 activity or expression and the lung cancer is identified as having intrinsic resistance to anti-PD1 therapy or anti-PD-L1 therapy. In some embodiments, provided is a method of treating a subject having, or at risk of developing, a lung cancer, the method comprising administering to the subject an effective amount of a histone deacetylase inhibitor, wherein the lung cancer is identified as having modified STK11 activity or expression and the lung cancer is identified as having acquired resistance to anti-PD1 therapy or anti-PD-L1 therapy.

In some embodiments, provided is a method of treating a subject having, or at risk of developing, a lung cancer, the method comprising administering to the subject an effective amount of a selective histone deacetylase inhibitor in combination with a second therapeutic agent, wherein the lung cancer is identified as having modified STK11 activity or expression. In some embodiments, provided is a method of treating a subject having, or at risk of developing, a lung cancer, the method comprising administering to the subject an effective amount of a selective histone deacetylase inhibitor in combination with an immune checkpoint modulator, wherein the lung cancer is identified as having modified STK11 activity or expression. In some embodiments, provided is a method of treating a subject having, or at risk of developing, a lung cancer, the method comprising administering to the subject an effective amount of a selective histone deacetylase inhibitor in combination with an anti-PD1 agent or an anti-PD-L1 agent, wherein the lung cancer is identified as having modified STK11 activity or expression. In some embodiments, provided is a method of treating a subject having, or at risk of developing, a lung cancer, the method comprising administering to the subject an effective amount of a selective histone deacetylase inhibitor in combination with an anti-PD1 agent or an anti-PD-L1 agent, wherein the lung cancer is identified as having modified STK11 activity or expression, wherein the anti-PD1 agent or the anti-PD-L1 agent is selected from the group consisting of nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab, BMS936559; durvalumab; avelumab; envafolimab; cosibelimab; sugemalimab, AUNP-12; atezolizumab and CA-170. In some embodiments, provided is a method of treating a subject having, or at risk of developing, a lung cancer, the method comprising administering to the subject an effective amount of a selective histone deacetylase inhibitor in combination with an anti-PD1 therapy or an anti-PD-L1 therapy, wherein the lung cancer is identified as having modified STK11 activity or expression.

In some embodiments, provided is a method of treating a subject having, or at risk of developing, a lung cancer, the method comprising administering to the subject an effective amount of an HDAC Class I-selective histone deacetylase inhibitor in combination with a second therapeutic agent, wherein the lung cancer is identified as having modified STK11 activity or expression. In some embodiments, provided is a method of treating a subject having, or at risk of developing, a lung cancer, the method comprising administering to the subject an effective amount of an HDAC Class I-selective inhibitor in combination with an immune checkpoint modulator, wherein the lung cancer is identified as having modified STK11 activity or expression. In some embodiments, provided is a method of treating a subject having, or at risk of developing, a lung cancer, the method comprising administering to the subject an effective amount of an HDAC Class I-selective inhibitor in combination with an anti-PD1 agent or an anti-PD-L1 agent, wherein the lung cancer is identified as having modified STK11 activity or expression. In some embodiments, provided is a method of treating a subject having, or at risk of developing, a lung cancer, the method comprising administering to the subject an effective amount of an HDAC Class I-selective inhibitor in combination with an anti-PD1 agent or an anti-PD-L1 agent, wherein the lung cancer is identified as having modified STK11 activity or expression, wherein the anti-PD1 agent or the anti-PD-L1 agent is selected from the group consisting of nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab. BMS936559; durvalumab; avelumab; envafolimab; cosibelimab; sugemalimab, AUNP-12; atezolizumab and CA-170. In some embodiments, provided is a method of treating a subject having, or at risk of developing, a lung cancer, the method comprising administering to the subject an effective amount of an HDAC Class I-selective inhibitor in combination with an anti-PD1 therapy or an anti-PD-L1 therapy, wherein the lung cancer is identified as having modified STK11 activity or expression.

In some embodiments, provided is a method of treating a subject having, or at risk of developing, a lung cancer, the method comprising administering to the subject an effective amount of an HDAC1-selective histone deacetylase inhibitor in combination with a second therapeutic agent, wherein the lung cancer is identified as having modified STK11 activity or expression. In some embodiments, provided is a method of treating a subject having, or at risk of developing, a lung cancer, the method comprising administering to the subject an effective amount of an HDAC1-selective inhibitor in combination with an immune checkpoint modulator, wherein the lung cancer is identified as having modified STK11 activity or expression. In some embodiments, provided is a method of treating a subject having, or at risk of developing, a lung cancer, the method comprising administering to the subject an effective amount of an HDAC1-selective inhibitor in combination with an anti-PD1 agent or an anti-PD-L1 agent, wherein the lung cancer is identified as having modified STK11 activity or expression. In some embodiments, provided is a method of treating a subject having, or at risk of developing, a lung cancer, the method comprising administering to the subject an effective amount of an HDAC1-selective inhibitor in combination with an anti-PD1 agent or an anti-PD-L1 agent, wherein the lung cancer is identified as having modified STK11 activity or expression, wherein the anti-PD1 agent or the anti-PD-L1 agent is selected from the group consisting of nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab, BMS936559; durvalumab; avelumab; envafolimab; cosibelimab; sugemalimab, AUNP-12; atezolizumab and CA-170. In some embodiments, provided is a method of treating a subject having, or at risk of developing, a lung cancer, the method comprising administering to the subject an effective amount of an HDAC1-selective inhibitor in combination with an anti-PD1 therapy or an anti-PD-L1 therapy, wherein the lung cancer is identified as having modified STK11 activity or expression.

In some embodiments, provided is a method of treating a subject having, or at risk of developing, a lung cancer, the method comprising administering to the subject an effective amount of an HDAC1,2-selective histone deacetylase inhibitor in combination with a second therapeutic agent, wherein the lung cancer is identified as having modified STK11 activity or expression. In some embodiments, provided is a method of treating a subject having, or at risk of developing, a lung cancer, the method comprising administering to the subject an effective amount of an HDAC1,2-selective inhibitor in combination with an immune checkpoint modulator, wherein the lung cancer is identified as having modified STK11 activity or expression. In some embodiments, provided is a method of treating a subject having, or at risk of developing, a lung cancer, the method comprising administering to the subject an effective amount of an HDAC1,2-selective inhibitor in combination with an anti-PD1 agent or an anti-PD-L1 agent, wherein the lung cancer is identified as having modified STK11 activity or expression. In some embodiments, provided is a method of treating a subject having, or at risk of developing, a lung cancer, the method comprising administering to the subject an effective amount of an HDAC1,2-selective inhibitor in combination with an anti-PD1 agent or an anti-PD-L1 agent, wherein the lung cancer is identified as having modified STK11 activity or expression, wherein the anti-PD1 agent or the anti-PD-L1 agent is selected from the group consisting of nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab, BMS936559; durvalumab; avelumab; envafolimab; cosibelimab; sugemalimab, AUNP-12; atezolizumab and CA-170. In some embodiments, provided is a method of treating a subject having, or at risk of developing, a lung cancer, the method comprising administering to the subject an effective amount of an HDAC1,2-selective inhibitor in combination with an anti-PD1 therapy or an anti-PD-L1 therapy, wherein the lung cancer is identified as having modified STK11 activity or expression.

In some embodiments, provided is a method of treating a subject having, or at risk of developing, a lung cancer, the method comprising administering to the subject an effective amount of a CoREST-selective deacetylase inhibitor in combination with a second therapeutic agent, wherein the lung cancer is identified as having modified STK11 activity or expression. In some embodiments, provided is a method of treating a subject having, or at risk of developing, a lung cancer, the method comprising administering to the subject an effective amount of a CoREST-selective deacetylase inhibitor in combination with an immune checkpoint modulator, wherein the lung cancer is identified as having modified STK11 activity or expression. In some embodiments, provided is a method of treating a subject having, or at risk of developing, a lung cancer, the method comprising administering to the subject an effective amount of a CoREST-selective deacetylase inhibitor in combination with an anti-PD1 agent or an anti-PD-L1 agent, wherein the lung cancer is identified as having modified STK11 activity or expression. In some embodiments, provided is a method of treating a subject having, or at risk of developing, a lung cancer, the method comprising administering to the subject an effective amount of a CoREST-selective deacetylase inhibitor in combination with an anti-PD1 agent or an anti-PD-L1 agent, wherein the lung cancer is identified as having modified STK11 activity or expression, wherein the anti-PD1 agent or the anti-PD-L1 agent is selected from the group consisting of nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab, BMS936559; durvalumab; avelumab; envafolimab; cosibelimab; sugemalimab, AUNP-12; atezolizumab and CA-170. In some embodiments, provided is a method of treating a subject having, or at risk of developing, a lung cancer, the method comprising administering to the subject an effective amount of a CoREST-selective deacetylase inhibitor in combination with an anti-PD1 therapy or an anti-PD-L1 therapy, wherein the lung cancer is identified as having modified STK11 activity or expression.

In some of the embodiments described herein, the combination of the HDAC inhibitor (e.g., selective HDAC inhibitor, HDAC1 selective inhibitor, HDAC1,2 selective inhibitor, class I selective HDAC inhibitor, CoREST complex selective HDAC inhibitor) and an immune checkpoint modulator is synergistic.

In some embodiments described herein, the method comprises identifying the subject as having one or more cancer cells that have modified STK11 activity or expression. In some embodiments described herein, the method comprises identifying the subject as having one or more cancer cells that have modified STK11 activity or expression and are resistant to anti-PD1 therapy or anti-PD-L1 therapy.

In some embodiments described herein, the method comprises administering to the subject an immune checkpoint modulator and an HDAC inhibitor, wherein the treatment modulates and/or improves the Teff cell to Treg cell ratio in the tumor or tumor microenvironment.

In some embodiments described herein, the method comprises administering to the subject an immune checkpoint modulator and an HDAC inhibitor, wherein the treatment reduces or depletes Treg cells in the tumor or tumor microenvironment.

In some embodiments described herein, the method comprises administering to the subject an immune checkpoint modulator and an HDAC inhibitor, wherein the treatment induces or increases the expression of cytokines that promote anti-tumor activity. T In some embodiments, the method further comprises the cytokines are selected from the group of CXCL9, CXCL10, and CXCL11.

In some embodiments described herein, the method comprises administering to the subject an immune checkpoint modulator and an HDAC inhibitor, wherein the treatment reduces the expression of cytokines that promote Treg cell recruitment. In some embodiments, the cytokines are CCL1 or CCL22.

In some embodiments described herein, the method comprises administering to the subject an immune checkpoint modulator and an HDAC inhibitor, wherein the administering of the HDAC inhibitor does not substantially reduce erythroid or myeloid cell viability (e.g., reduces the cell viability by less than 10%, by less than 20%, by less than 30%, by less than 40% or by less than 50%).

In some embodiments described herein, the cancer presents an immune evasion phenotype characterized by STK11 mutant expression comprising: administering an HDAC1,2 selective inhibitor, wherein the HDAC1,2 selective inhibitor is capable of attenuating or reversing the immune evasion phenotype. In some embodiments, the method further comprises administering an immune checkpoint modulator.

In some embodiments described herein, the method comprises administering to the subject an immune checkpoint modulator and an HDAC inhibitor, wherein the treatment results in increased IFNgamma expression in the tumor or tumor microenvironment.

In some embodiments, provided is a method of treating a subject having, or at risk of developing, an immune evasive cancer, the method comprising administering to the subject an effective amount of a histone deacetylase inhibitor, wherein the immune evasive cancer is identified as having modified STK11 activity or expression. In some embodiments, provided is a method of treating a subject having, or at risk of developing, an immune evasive cancer, the method comprising administering to the subject an effective amount of a histone deacetylase inhibitor in combination with a second therapeutic agent, wherein the immune evasive cancer is identified as having modified STK11 activity or expression. In some embodiments, the second therapeutic agent is an immune checkpoint modulator as described herein.

In some embodiments, provided is a method of reversing immune evasion in a subject having, or at risk of developing, a cancer, the method comprising administering to the subject an effective amount of a histone deacetylase inhibitor, wherein the cancer is identified as having modified STK11 activity or expression. In some embodiments, provided is a method of reversing immune evasion in a subject having, or at risk of developing, a cancer, the method comprising administering to the subject an effective amount of a histone deacetylase inhibitor in combination with a second therapeutic agent, wherein the cancer is identified as having modified STK11 activity or expression. In some embodiments, provided is a method of reversing immune evasion in a subject having, or at risk of developing, a cancer, the method comprising administering to the subject an effective amount of a histone deacetylase inhibitor in combination with a second therapeutic agent, wherein the cancer is identified as having modified STK11 activity or expression and the immune evasion is caused by anti-PD1 therapy or anti-PD-L1 therapy. In some embodiments, the second therapeutic agent is an immune checkpoint modulator as described herein.

In some embodiments described herein, the subject has a cancer. In some embodiments, the subject is at risk of developing a cancer.

In some embodiments described herein, the cancer is lung cancer (e.g., lung adenocarcinoma, non-small cell lung cancer (NSCLC), squamous cell lung carcinoma), colorectal cancer (e.g., colon adenocarcinoma, rectal adenocarcinoma), breast cancer (e.g., invasive ductal carcinoma), pancreatic cancer (e.g., pancreatic adenocarcinoma), endometrial cancer (e.g., endometrioid carcinoma), neuroendocrine cancer (e.g., large cell neuroendocrine carcinoma), melanoma, non-melanoma skin cancer (e.g., skin squamous cell carcinoma), cholangiocarcinoma, gallbladder cancer, ovarian cancer (e.g., ovarian serous adenocarcinoma), bladder cancer (e.g., bladder urothelial carcinoma), prostate cancer (e.g., prostate adenocarcinoma), cervical cancer, endocervical cancer or cancer of unknown primary (e.g., adenocarcinoma of unknown primary).

In some embodiments described herein, the cancer is lung cancer (e.g., lung adenocarcinoma, non-small cell lung cancer (NSCLC)), colorectal cancer (e.g., colon adenocarcinoma), pancreatic cancer (e.g., pancreatic adenocarcinoma), endometrial cancer (e.g., endometrioid carcinoma), melanoma, non-melanoma skin cancer (e.g., skin squamous cell carcinoma), cholangiocarcinoma, ovarian cancer (e.g., ovarian serous adenocarcinoma), cervical cancer, endocervical cancer or cancer of unknown primary (e.g., adenocarcinoma of unknown primary).

In some embodiments described herein, the cancer is lung cancer (e.g., lung adenocarcinoma, non-small cell lung cancer (NSCLC)), colorectal cancer (e.g., colon adenocarcinoma), pancreatic cancer (e.g., pancreatic adenocarcinoma), endometrial cancer (e.g., endometrioid carcinoma), melanoma, non-melanoma skin cancer (e.g., skin squamous cell carcinoma), cholangiocarcinoma, ovarian cancer (e.g., ovarian serous adenocarcinoma) or cancer of unknown primary (e.g., adenocarcinoma of unknown primary).

In some embodiments described herein, the cancer is lung cancer. In some embodiments described herein, the cancer is lung adenocarcinoma. In some embodiments described herein, the cancer is non-small cell lung cancer (NSCLC).

In some embodiments described herein, the cancer is colon cancer. In some embodiments described herein, the cancer is colon adenocarcinoma. In some embodiments described herein, the cancer is colorectal cancer.

In some embodiments described herein, the cancer is breast cancer (e.g., invasive ductal carcinoma). In some embodiments described herein, the cancer is pancreatic cancer (e.g., pancreatic adenocarcinoma). In some embodiments described herein, the cancer is endometrial cancer (e.g., endometrioid carcinoma). In some embodiments described herein, the cancer is neuroendocrine cancer (e.g., large cell neuroendocrine carcinoma). In some embodiments described herein, the cancer is melanoma. In some embodiments described herein, the cancer is non-melanoma skin cancer (e.g., skin squamous cell carcinoma). In some embodiments described herein, the cancer is cholangiocarcinoma. In some embodiments described herein, the cancer is gallbladder cancer. In some embodiments described herein, the cancer is ovarian cancer (e.g., ovarian serous adenocarcinoma). In some embodiments described herein, the cancer is bladder cancer (e.g., bladder urothelial carcinoma). In some embodiments described herein, the cancer is prostate cancer (e.g., prostate adenocarcinoma). In some embodiments described herein, the cancer is cervical cancer. In some embodiments described herein, the cancer is endocervical cancer. In some embodiments described herein, the cancer is cancer of unknown primary (e.g., adenocarcinoma of unknown primary).

In some of the embodiments described herein (e.g., in this section), the cancer has increased or decreased STK11 expression. In one embodiment, the increased or decreased STK expression is assessed by determining copy number of the gene encoding STK11 relative to a control sample, wherein an increase in the copy number indicates an increased level of expression and a decrease in the copy number indicates a decreased level of expression. In one embodiment, the increased or decreased STK expression is assessed by determining the level of STK11 protein or mRNA relative to a control sample. In one embodiment, the cancer has decreased STK11 expression.

In some of the embodiments described herein (e.g., in this section), the cancer has a STK11 mutation. In one embodiment, the STK11 mutation is a mutation selected from: mutation selected from: (i) a mutation in the nucleotide sequence encoding STK11; (ii) a mutation in a regulatory sequence controlling expression of the nucleotide sequence encoding STK11; (iii) a mutation in a nucleotide encoding a protein which interacts with the transcription product of the STK11 gene; (iv) a mutation in the translation product of the STK11 gene; and (v) a mutation in the transcription product of the STK11 gene.

In one embodiment, the STK11 mutation is a mutation selected from (i) a mutation in the nucleotide sequence encoding STK11: (ii) a mutation in a regulatory sequence controlling expression of the nucleotide sequence encoding STK11; and (iii) a mutation in a nucleotide encoding a protein which interacts with the transcription product of the STK11 gene.

In one embodiment, the STK11 mutation a mutation in the nucleotide sequence encoding STK11 In one embodiment, the STK11 mutation is a mutation in the translation product of the STK11 gene. In one embodiment, the STK11 mutation is a mutation in the transcription product of the STK11 gene. In one embodiment, the STK11 mutation is an inactivating (loss of function) mutation.

In some of the embodiments described herein (e.g., in this section), the cancer is resistant to anti-PD 1 therapy or anti-PD-L1 therapy. In one embodiment, the cancer has intrinsic resistance to anti-PD1 therapy or anti-PD-L1 therapy. In one embodiment, the cancer has acquired resistance to anti-PD1 therapy or anti-PD-L1 therapy.

In some of the embodiments described herein (e.g., in this section), the cancer is resistant to chemotherapy (e.g., platinum-containing chemotherapy). In one embodiment, the cancer has intrinsic resistance to chemotherapy (e.g., platinum-containing chemotherapy). In one embodiment, the cancer has acquired resistance to chemotherapy (e.g., platinum-containing chemotherapy).

In some of the embodiments described herein (e.g., in this section), the cancer does not respond to or benefit from treatment with an immune checkpoint modulator when administered alone or as part of a treatment regimen that does not include an HDAC inhibitor.

### “Subject”

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgous monkeys, rhesus monkeys), cattle, pigs, horses, sheep, goats, rodents, cats, and/or dogs. In some embodiments, the subject is a human. In certain embodiments, the subject is a non-human animal. The terms “human.” “patient,” and “subject” may be used interchangeably herein, as context permits.

### “Treating”

As used herein, and unless otherwise specified, the terms “treat,” “treating” and “treatment” contemplate an action that occurs while a subject is suffering from the specified disease, disorder or condition (e.g., cancer), which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”). In some embodiments, provided herein are contemplated methods of therapeutic treatment wherein the action occurs while a subject is suffering from the specified disease, disorder or condition (e.g., cancer) and results in a reduction in the severity of the disease, disorder or condition, or retardation or slowing of the progression of the disease, disorder or condition. In an alternate embodiment, provided herein are methods of prophylactic treatment wherein the action occurs before a subject begins to suffer from the specified disease, disorder or condition (e.g., cancer) and results in preventing a disease, disorder or condition, or one or more symptoms associated with the disease, disorder or condition, or preventing the recurrence of the disease, disorder or condition.

### Combination Therapy

Provided herein are methods of treatment of diseases or disorders (e.g., cancers with modified STK11 activity or expression) HDAC inhibitors in combination with one or more additional therapeutic agents.

The term “Combination” refers to either a fixed combination in one dosage unit form, or a combined administration where a compound described herein (e.g., HDAC inhibitor) and a combination partner (e.g., another drug as explained below, also referred to as “additional therapeutic agent” or “co-agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g., a compound described herein (e.g., HDAC inhibitor) and a combination partner, are both administered to a patient simultaneously in the form of a single entity or dosage. The term “non-fixed combination” means that the therapeutic agents, e.g., a compound described herein (e.g., HDAC inhibitor) and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more therapeutic agent.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times.

In certain embodiments, compounds described herein (e.g., HDAC inhibitor) are combined with other therapeutic agents, including, but not limited to, immune checkpoint modulators and other immunotherapies, other anti-cancer agents (e.g., chemotherapeutic agents, targeted agents), anti-allergic agents, anti-nausea agents (or anti-emetics), pain relievers, cytoprotective agents, radiation therapy and combinations thereof.

In some embodiments, the combination therapy modulates the ratio of Tem to Treg cells. In some embodiments, the combination therapy increases the ratio of Tem to Treg cells in the tumor or the tumor microenvironment. Effector memory T cells (Tem) express CD45RO but lack expression of CCR7 and CD62L. They also have intermediate to high expression of CD44. CD62L acts as a “homing receptor” for lymphocytes to enter secondary lymphoid tissues. Thus, Tem cells are typically found in the peripheral circulation and tissues, rather than in the lymph nodes, and exhibit immediate effector function. In response to antigen stimulation, Tem cells proliferate and differentiate into CD62L⁻ effector T cells. Effector T cells (Teff) are fully differentiated T cells. Effector T cells are short-lived cells, as opposed to memory cells which have a potential of long-term survival but have strong cytotoxic activity.

Regulatory T cells (Tregs) are a specialized subpopulation of T cells that act to suppress immune response, thereby maintaining homeostasis and self-tolerance. Tregs are able to inhibit T cell proliferation and cytokine production and play a critical role in preventing autoimmunity.

In some embodiments, the combination therapy modulates cytokine secretion in the tumor or tumor microenvironment. In some embodiments, the cytokine is selected from the group of CXCL9, CXCL10, and CXCL11. In some embodiments, the expression of CXCL9, CXCL10, and/or CXCL11 increases. In some embodiments, the cytokine is selected from the group of CCL1 and CCL22. In some embodiments, the expression of CCL1 and/or CCL22 decreases. In some embodiments, the combination therapy modulates IFNgamma expression and/or secretion in the tumor or tumor microenvironment. In some embodiments, the IFNgamma expression and/or secretion is increased.

### Immunotherapies

In some embodiments, at least one of the other therapeutic agents is an immunotherapeutic agent. In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and one or more immunotherapeutic agents.

In some embodiments, the immunotherapeutic agent is a cell-based therapy. In some embodiments, provided is a method of treating a disease or disorder (e.g., cancer having modified STK11 activity or expression) comprising administering or coadministering, in any order, to a patient in need thereof, a compound described herein (e.g., HDAC inhibitor) and an adoptive cell-based therapy. In some embodiments, the adoptive cell-based therapy is a CAR-T therapy or a TIL therapy. In some embodiments, the adoptive cell-based therapy is a CAR-T therapy. In some embodiments, the adoptive cell-based therapy is a TIL therapy.

In some embodiments, the immunotherapeutic agent is a cancer vaccine such as a neoantigen. These vaccines can be developed using peptides or RNA, In some embodiments, the immunotherapeutic agent is an oncolytic virus. In some embodiments, the immunotherapeutic agent is a STING pathway agonist. Exemplary STING agonists include MK-1454 and ADU-S100.

In some embodiments, the immunotherapeutic agent is an immune checkpoint modulator as described herein.

### Immune Checkpoint Modulators

As used herein, an “immune checkpoint modulator” is an agent that modulates the immune checkpoint pathway, either by blocking any inhibitory immune checkpoint protein or by activating any stimulatory immune checkpoint protein. Unless otherwise specified, references to immune checkpoint modulators in the methods and uses described herein refer to any of the immune checkpoint modulators described herein (e.g., in this section).

In one embodiment, the immune checkpoint modulator is a T cell co-stimulatory receptor agonist or a dendritic cell co-stimulatory receptor agonist. In one embodiment, the immune checkpoint modulator is a T cell co-stimulatory receptor agonist. In one embodiment, the immune checkpoint modulator is a dendritic cell co-stimulatory receptor agonist.

In some embodiments, the immune checkpoint modulator is an immune checkpoint inhibitor. In some embodiments, the immune checkpoint inhibitor is an antibody.

In one embodiment, the immune checkpoint modulator is a co-stimulatory antibody (e.g., anti-4-1BB antibody, anti-OX40 antibody, anti-GITR antibody, anti-CD28 antibody, anti-CD27 antibody, anti-ICOS antibody, anti-CD40 antibody). In one embodiment, the immune checkpoint modulator is an anti-4-1BB antibody. In one embodiment, the immune checkpoint modulator is an anti-OX40 antibody. In one embodiment, the immune checkpoint modulator is an anti-GITR antibody. In one embodiment, the immune checkpoint modulator is an anti-CD28 antibody. In one embodiment, the immune checkpoint modulator is an anti-CD27 antibody. In one embodiment, the immune checkpoint modulator is an anti-ICOS antibody. In one embodiment, the immune checkpoint modulator is an anti-CD40 antibody.

In one embodiment, the immune checkpoint modulator is an anti-CTLA agent. In one embodiment, the immune checkpoint modulator is an anti-CTLA-4 antibody (e.g., ipilimumab, tremelimumab). In one embodiment, the immune checkpoint modulator is ipilimumab. In one embodiment, the immune checkpoint modulator is tremelimumab.

Anti-PD-1/PD-L1 therapies aim to block the activity of PD-1 and PDL1 immune checkpoint proteins, preventing the transmission of an “off” signal to T cells, thus allowing the T cells to infiltrate and destroy tumors. Anti-PD-1/PD-L1 agents prevent the association of the programmed death-ligand 1 (PD-L1) with its receptor, programmed cell death protein 1 (PD-1). Anti-PD-1 agents bind to the PD-1 protein, whereas anti-PD-L1 agents bind to the PD-L1 ligand.

In one embodiment, the immune checkpoint modulator is a PD-1 ligand (i.e., PD-LI, B7-HI or CD274) or PD-2 ligand (i.e., PD-L2, B7-DC or CD273)).

In one embodiment, the immune checkpoint modulator is an anti-PD-1 agent (PD-1 inhibitor). In one embodiment, the immune checkpoint modulator is an anti-PD-1 antibody (e.g., nivolumab (i.e., MDX-1106, BMS-936558, ONO-4538): AMP-224: pembrolizumab (MK-3475); pidilizumab (CT-011), cemiplimab; dostarlimab; prolgolimab; spartalizumab; camrelizumab; sasanlimab, sintilimab; tislelizumab; toripalimab; retifanlimab; MEDI0680; budigalimab; geptanolimab). In one embodiment, the immune checkpoint modulator is nivolumab. In one embodiment, the immune checkpoint modulator is pembrolizumab. In one embodiment, the immune checkpoint modulator is pidilizumab. In one embodiment, the immune checkpoint inhibitor is cemiplimab. In one embodiment, the immune checkpoint inhibitor is dostarlimab. In one embodiment, the immune checkpoint inhibitor is prolgolimab. In one embodiment, the immune checkpoint inhibitor is spartalizumab. In one embodiment, the immune checkpoint inhibitor is camrelizumab. In one embodiment, the immune checkpoint inhibitor is sasanlimab, sintilimab. In one embodiment, the immune checkpoint inhibitor is tislelizumab. In one embodiment, the immune checkpoint inhibitor is toripalimab. In one embodiment, the immune checkpoint inhibitor is retifanlimab. In one embodiment, the immune checkpoint inhibitor is MEDI0680. In one embodiment, the immune checkpoint inhibitor is budigalimab. In one embodiment, the immune checkpoint inhibitor is geptanolimab.

In one embodiment, the immune checkpoint modulator is an anti-PD-L1 agent (PD-L1 inhibitor). In one embodiment, the immune checkpoint modulator is an anti-PD-L1 antibody (e.g., BMS936559 (i.e., MDX-1105); durvalumab (MEDI4736); avelumab (MSB0010718C) envafolimab; cosibelimab; sugemalimab. AUNP-12; or atezolizumab (MPDL-3280A). In one embodiment, the immune checkpoint modulator is durvalumab. In one embodiment, the immune checkpoint modulator is atezolizumab. In one embodiment, the immune checkpoint modulator is avelumab. In one embodiment, the immune checkpoint modulator is envafolimab. In one embodiment, the immune checkpoint modulator is cosibelimab. In one embodiment, the immune checkpoint modulator is sugemalimab. In one embodiment, the immune checkpoint modulator is AUNP-12. In one embodiment, the immune checkpoint inhibitor is anti-PD-L1 small molecule (e.g., CA-170).

In one embodiment, the immune checkpoint modulator is a checkpoint co-inhibitory antibody (e.g., anti-TIM3, anti-LAG3, Eftilagimod alpha (IMP321), anti-TIGIT, anti B7-H3 (e.g., enoblituzumab (MGA271)).

In one embodiment, the immune checkpoint modulator is an anti-TWEAKR antibody, an anti-HVEM antibody, an anti-TIM-1 antibody, or an anti-VISTA antibody.

In one embodiment, provided is a method of treating a subject having, or at risk of developing, a cancer, wherein the cancer is identified as having modified STK11 activity or expression, the method comprising administering to the subject an effective amount of a histone deacetylase (HDAC) inhibitor and one or more immune checkpoint modulators independently selected from an anti-CTLA-4 antibody, an anti-PD-1 antibody, an anti-PD-L1 antibody, an anti-4-1 BB antibody, an anti-OX-40 antibody, an anti-GITR antibody, an anti-CD27 antibody, an anti-CD28 antibody, an anti-CD40 antibody, an anti-LAG3 antibody, an anti-ICOS antibody, an anti-TWEAKR antibody, an anti-HVEM antibody, an anti-TIM-1 antibody, an anti-TIM-3 antibody, an anti-VISTA antibody, and an anti-TIGIT antibody.

### Chemotherapy

In one embodiment, at least one of the other therapeutic agents is a chemotherapeutic agent.

Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BICNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®), Rubex®), etoposide (Vepesid®), fludarabine phosphate (Fludara®), 5-fluorouracil (Adrucil®), Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), pemetrexed (Alimta®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

In some embodiments, each chemotherapeutic agent is independently selected from cisplatin (Platinol®), carboplatin (Paraplatin®), paclitaxel (Taxol®), nab-paclitaxel (Abraxane®), docetaxel (Taxotere®), Gemcitabine (difluorodeoxycitidine), vinorelbine (Navelbine®), etoposide (Vepesid®), epemetrexed (Alimta®).

In one embodiment, at least one chemotherapeutic agent is a platinum containing therapeutic agent (e.g., cisplatin or carboplatin). In one embodiment, at least one chemotherapeutic agent is cisplatin. In one embodiment, one chemotherapeutic agent is a platinum-containing chemotherapeutic agent, and a second chemotherapeutic agent is pemetrexed.

### Targeted Therapy

In one embodiment, at least one of the other therapeutic agents is a targeted agent.

In one embodiment, each targeted agent is independently selected from an anti-angiogenesis agent (e.g., an anti-VEGF agent), a KRAS inhibitor, an ALK inhibitor, a ROS1 inhibitor, a BRAF inhibitor, a RET inhibitor, a MEK inhibitor, a MET inhibitor and a TRK inhibitor.

In one embodiment, each targeted agent is independently selected from bevacizumab, ramucirumab, sotorasib, crizotinib, ceritinib, alectinib, brigatinib, lorlatinib, entrectinib, dabrafenib, trametinib, capmatinib, tepotinib and larotrectinib

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and a KRAS inhibitor. In some embodiments, the KRAS inhibitor is a KRAS^{G12C} inhibitor (e.g., sotorasib, adagrasib, ARS-3248, LY3499446, LY3537982, GDC-6036, D3ₛ-001, D-1553, JDQ443, BI 1823911, RMC-6291, GFH925, JAB-21822, BPI-421286, HBI-2438). In some embodiments, the KRAS^{G12C} inhibitor is sotorasib. In some embodiments, the KRAS inhibitor is a KRAS^{G12D} inhibitor (e.g., MRTX1133, RMC-9805). In some embodiments, the KRAS inhibitor is a KRAS^{Q61H} inhibitor (e.g., RMC-0708). In some embodiments, the KRAS inhibitor is a KRAS^{G13C} inhibitor (e.g., RMC-8839). In some embodiments, the KRAS inhibitor is a pan-KRAS inhibitor (e.g., RMC-6236, BI 1701963).

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and an HDM2 inhibitor and/or with 5-FU.

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and a CDK4 inhibitor, including, but not limited to, LEE011 or a CDK 4/6 inhibitor (e.g., palbociclib (Ibrance®), ribociclib (Kisqali®), and abemaciclib (Verzenio®).

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and targeted treatments contingent on the dependency of individual target tumors on relevant pathways as determined by suitable predictive markers, including but not limited to: inhibitors of HDM2i, PI3K/mTOR-I, MAPKi, RTKi (FGFRi, METi, IGFiRi, JAKi, and WNTi).

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and a disease-specific huMABs (e.g., an anti-HER3 huMAB).

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and ADCs/ADCCs contingent on the expression of relevant surface targets on target tumors of interest.

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and a CAAP1 inhibitor.

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and a AKAP17A inhibitor.

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and a BCL2L1 inhibitor. In some embodiments, the BCL2L1 inhibitor is AT-101.

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and a TSC1/2 inhibitor.

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and a UBE2H inhibitor.

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and a NF2 inhibitor.

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and a ZC3HC1 inhibitor.

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and a MGEA5 inhibitor.

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and a CNOT4 inhibitor.

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and a API5 inhibitor.

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and a HEXIM1 inhibitor.

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and a PTEN inhibitor.

In some embodiments, provided is a method of treating a disease or disorder (e.g., cancer having modified STK11 activity or expression) comprising administering or co-administering, in any order, to a patient in need thereof, a compound described herein (e.g., HDAC inhibitor) and a DNA damage pathway inhibitor. In some embodiments, the DNA damage pathway inhibitor is selected from the group consisting of bleomycin, an ATM inhibitor (e.g., AZD1390), a USP1 inhibitor, a WEE1 inhibitor (e.g., AZD1775), and a Chk1 inhibitor (e.g., AZD7762).

### Other Therapeutic Agents

Some patients may experience allergic reactions to the compounds described herein (e.g., HDAC inhibitor) and/or other anti-cancer agent(s) during or after administration; therefore, anti-allergic agents are often administered to minimize the risk of an allergic reaction. Suitable anti-allergic agents include corticosteroids, including, but not limited to, dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®), Orapred®, Pediapred®) and Prelone®), prednisone (sold under the tradenames Deltasone®), Liquid Red®, Meticorten® and Orasone®), methylprednisolone (also known as 6-methylprednisolone, methylprednisolone acetate, methylprednisolone sodium succinate, sold under the tradenames Duralone R, Medralone®) Medrol®, M-Prednisol® and Solu-Medrol®): antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®).

Some patients may experience nausea during and after administration of the compound described herein (e.g., HDAC inhibitor) and/or other anti-cancer agent(s); therefore, anti-emetics are used in preventing nausea (upper stomach) and vomiting. Suitable anti-emetics include aprepitant (Emend®), ondansetron (Zofran®), granisetron HCl (Kytril®), lorazepam (Ativan®, dexamethasone (Decadron®), prochlorperazine (Compazine®), casopitant (Rezonic® and Zunrisa®), and combinations thereof.

Medication to alleviate the pain experienced during the treatment period is often prescribed to make the patient more comfortable. Common over-the-counter analgesics, such Tylenol®, are often used. However, opioid analgesic drugs including, but not limited to, hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), and fentanyl (e.g., Duragesic®) are also useful for moderate or severe pain.

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. Suitable cytoprotective agents include Amifostine (Ethyol®), glutamine, dimesna (Tavocept®), mesna (Mesnex®), dexrazoxane (ZinecardR or Totect®), xaliproden (Xaprila®), and leucovorin (also known as calcium leucovorin, citrovorum factor and folinic acid).

The structure of the active compounds identified by code numbers, generic or trade names may be taken from the actual edition of the standard compendium “The Merck Index” or from databases, e.g., Patents International (e.g., IMS World Publications).

The above-mentioned compounds, which can be used in combination with a compound of described herein (e.g., HDAC inhibitor), can be prepared and administered as described in the art, including, but not limited to, in the documents cited herein.

In some embodiments, provided are pharmaceutical compositions comprising at least one compound described herein (e.g., HDAC inhibitor) together with a pharmaceutically acceptable carrier suitable for administration to a human or animal subject, either alone or together with other anti-cancer agents. In particular, compositions will either be formulated together as a combination therapeutic or administered separately.

In some embodiments, provided is a method of treating a subject having, or at risk of developing, a cancer, the method comprising administering to the subject a combination of an HDAC inhibitor as described herein (e.g., selective HDAC inhibitor, HDAC1 selective inhibitor, HDAC1,2 selective inhibitor, class I selective HDAC inhibitor, CoREST complex selective HDAC inhibitor), an immune checkpoint modulator as described herein (e.g., an anti-CTLA-4 antibody, an anti-PD-1 antibody, an anti-PD-L1 antibody or a combination thereof) and an additional therapeutic agent as described herein (e.g., targeted agent, a chemotherapeutic agent, radiation or a combination thereof).

In combination therapy, the compound described herein (e.g., HDAC inhibitor) and other anti-cancer agent(s) may be administered either simultaneously, concurrently or sequentially with no specific time limits, wherein such administration provides therapeutically effective levels of the two compounds in the body of the patient.

In one embodiment, the HDAC inhibitor is administered simultaneously with the additional therapeutic agents.

In one embodiment, the HDAC inhibitor is administered sequentially with the additional therapeutic agents.

In one embodiment, the HDAC inhibitor is administered prior to the additional therapeutic agents.

In one embodiment, the HDAC inhibitor is administered subsequent to the additional therapeutic agents.

In one embodiment, the method comprises administering to the subject an immune checkpoint modulator followed by administering to the subject an HDAC inhibitor.

In one embodiment, the method comprises administering to the subject an HDAC inhibitor followed by administering to the subject an immune checkpoint modulator.

In one embodiment, the method comprises administering to the subject an immune checkpoint modulator, followed by administering to the subject a targeted agent, a chemotherapeutic agent, radiation or a combination thereof, followed by administering to the subject an HDAC inhibitor.

In one embodiment, the method comprises administering to the subject an immune checkpoint modulator, followed by administering to the subject an HDAC inhibitor, followed by administering to the subject a targeted agent, a chemotherapeutic agent, radiation or a combination thereof.

In one embodiment, the method comprises administering to the subject an HDAC inhibitor, followed by administering to the subject a targeted agent, a chemotherapeutic agent, radiation or a combination thereof, followed by administering to the subject an immune checkpoint modulator.

In one embodiment, the method comprises administering to the subject an HDAC inhibitor, followed by administering to the subject an immune checkpoint modulator, followed by administering to the subject a targeted agent, a chemotherapeutic agent, radiation or a combination thereof.

In one embodiment, the method comprises administering to the subject a targeted agent, a chemotherapeutic agent, radiation or a combination thereof, followed by administering to the subject an immune checkpoint modulator, followed by administering to the subject an HDAC inhibitor.

In one embodiment, the method comprises administering to the subject a targeted agent, a chemotherapeutic agent, radiation or a combination thereof, followed by administering to the subject an HDAC inhibitor, followed by administering to the subject an immune checkpoint modulator.

In some embodiments, the compound described herein (e.g., HDAC inhibitor) and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound described herein (e.g., HDAC inhibitor) and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In some embodiments, provided are kits that include one or more compound described herein (e.g., HDAC inhibitor) and a second therapeutic agent as disclosed herein are provided. Representative kits include (a) a compound described herein (e.g., HDAC inhibitor) and (b) at least one other therapeutic agent, e.g., as indicated above, whereby such kit may comprise a package insert or other labeling including directions for administration.

A compound described herein (e.g., HDAC inhibitor) may also be used in combination with known therapeutic processes, for example, the administration of hormones or especially radiation.

### Patient Selection and Monitoring

### Patient Selection for Treatment

In one embodiment, provided is a method of selecting a subject for treatment with an HDAC inhibitor the method comprising: identifying subjects having a cancer characterized by the presence of cells having modified STK11 activity or expression; and selecting thus identified subjects for treatment with one of the methods of treatment described herein.

In one embodiment, provided is a method of selecting a subject for treatment with an HDAC inhibitor the method comprising: identifying subjects having a cancer characterized by the presence of cells having modified STK11 activity or expression; and selecting thus identified subjects for treatment with an HDAC inhibitor.

In one embodiment, provided is a method of selecting a subject for treatment with a combination of an HDAC inhibitor and one or more additional therapeutic agents, the method comprising:

identifying subjects having a cancer characterized by the presence of cells having modified STK11 activity or expression; and selecting thus identified subjects for treatment with an HDAC inhibitor and one or more additional therapeutic agents.

In one embodiment, provided is a method of selecting a subject for treatment with a combination of an HDAC inhibitor and one or more additional therapeutic agents, the method comprising: identifying subjects having a cancer characterized by the presence of cells having modified STK11 activity or expression; and selecting thus identified subjects for treatment with an HDAC inhibitor and one or more immune checkpoint modulators.

In one embodiment, provided is a method of selecting a subject for treatment with a combination of an HDAC inhibitor and two or more additional therapeutic agents, the method comprising: identifying subjects having a cancer characterized by the presence of cells having modified STK11 activity or expression; and selecting thus identified subjects for treatment with an HDAC inhibitor and two or more additional therapeutic agents, wherein at least two of the additional therapeutic agents are immune checkpoint modulators.

In one embodiment, provided is a method of selecting a subject for treatment with a combination of an HDAC inhibitor, an immune checkpoint modulator and one or more additional therapeutic agents selected from a chemotherapeutic agent, a targeted agent and radiotherapy, the method comprising: identifying subjects that have previously been treated with a combination of an immune checkpoint modulator and one or more additional therapeutic agents selected from a chemotherapeutic agent, a targeted agent and radiotherapy, wherein treatment with the combination of immune checkpoint modulator and additional therapeutic agent did not provide any additional benefit as compared to treatment with the additional therapeutic agent alone; and selecting thus identified subjects for treatment.

In one embodiment, the method further comprises: identifying subjects having a cancer characterized by the presence of cells having decreased STK11 activity or expression; and selecting thus identified subjects for treatment.

In some embodiments described herein, the cancer is lung cancer (e.g., lung adenocarcinoma, non-small cell lung cancer (NSCLC), squamous cell lung carcinoma), colorectal cancer (e.g., colon adenocarcinoma, rectal adenocarcinoma), breast cancer (e.g., invasive ductal carcinoma), pancreatic cancer (e.g., pancreatic adenocarcinoma), endometrial cancer (e.g., endometrioid carcinoma), neuroendocrine cancer (e.g., large cell neuroendocrine carcinoma), melanoma, non-melanoma skin cancer (e.g., skin squamous cell carcinoma), cholangiocarcinoma, gallbladder cancer, ovarian cancer (e.g., ovarian serous adenocarcinoma), bladder cancer (e.g., bladder urothelial carcinoma), prostate cancer (e.g., prostate adenocarcinoma), cervical cancer, endocervical cancer or cancer of unknown primary (e.g., adenocarcinoma of unknown primary).

In some embodiments described herein, the cancer is lung cancer (e.g., lung adenocarcinoma, non-small cell lung cancer (NSCLC)), colorectal cancer (e.g., colon adenocarcinoma), pancreatic cancer (e.g., pancreatic adenocarcinoma), endometrial cancer (e.g., endometrioid carcinoma), melanoma, non-melanoma skin cancer (e.g., skin squamous cell carcinoma), cholangiocarcinoma, ovarian cancer (e.g., ovarian serous adenocarcinoma), cervical cancer, endocervical cancer or cancer of unknown primary (e.g., adenocarcinoma of unknown primary).

In some embodiments described herein, the cancer is lung cancer (e.g., lung adenocarcinoma, non-small cell lung cancer (NSCLC)), colorectal cancer (e.g., colon adenocarcinoma), pancreatic cancer (e.g., pancreatic adenocarcinoma), endometrial cancer (e.g., endometrioid carcinoma), melanoma, non-melanoma skin cancer (e.g., skin squamous cell carcinoma), cholangiocarcinoma, ovarian cancer (e.g., ovarian serous adenocarcinoma) or cancer of unknown primary (e.g., adenocarcinoma of unknown primary).

In some embodiments described herein, the cancer is lung cancer. In some embodiments described herein, the cancer is lung adenocarcinoma. In some embodiments described herein, the cancer is non-small cell lung cancer (NSCLC).

In some embodiments described herein, the cancer is colon cancer. In some embodiments described herein, the cancer is colon adenocarcinoma. In some embodiments described herein, the cancer is colorectal cancer.

In some of the embodiments described herein (e.g., in this section), the cancer has increased or decreased STK11 expression. In one embodiment, the increased or decreased STK expression is assessed by determining copy number of the gene encoding STK11 relative to a control sample, wherein an increase in the copy number indicates an increased level of expression and a decrease in the copy number indicates a decreased level of expression. In one embodiment, the increased or decreased STK expression is assessed by determining the level of STK11 protein or mRNA relative to a control sample. In one embodiment, the cancer has decreased STK11 expression.

In some of the embodiments described herein (e.g., in this section), the cancer has a STK11 mutation. In one embodiment, the STK11 mutation is a mutation selected from: mutation selected from: (i) a mutation in the nucleotide sequence encoding STK11; (ii) a mutation in a regulatory sequence controlling expression of the nucleotide sequence encoding STK11; (iii) a mutation in a nucleotide encoding a protein which interacts with the transcription product of the STK11 gene; (iv) a mutation in the translation product of the STK11 gene; and (v) a mutation in the transcription product of the STK11 gene.

In one embodiment, the STK11 mutation is a mutation selected from (i) a mutation in the nucleotide sequence encoding STK11; (ii) a mutation in a regulatory sequence controlling expression of the nucleotide sequence encoding STK11; and (iii) a mutation in a nucleotide encoding a protein which interacts with the transcription product of the STK11 gene.

In one embodiment, the STK11 mutation a mutation in the nucleotide sequence encoding STK11 In one embodiment, the STK11 mutation is a mutation in the translation product of the STK11 gene. In one embodiment, the STK11 mutation is a mutation in the transcription product of the STK11 gene. In one embodiment, the STK11 mutation is an inactivating (loss of function) mutation.

In one embodiment, the cancer is further characterized by one or more additional mutations. In one embodiment, the additional mutations are selected from KRAS mutations and KEAP1 mutations. In one embodiment, the additional mutations are KRAS mutations. In one embodiment, the KRAS mutations are selected from G12C, G12D, G12V, G12A, G12S, G12R, G13C, G13D, G13S, Q61H and Q61K mutations. In one embodiment, the KRAS mutations are a mutations at position G12, optionally wherein the KRAS mutations are selected from G12D mutations, G12C mutations, G12V mutations or combinations thereof. In one embodiment, the KRAS mutations are activating mutations. In one embodiment, the additional mutations are KEAP1 mutations. In one embodiment, the KEAP1 mutations are inactivating mutations. In one embodiment, the additional mutations are KRAS mutations and KEAP1 mutations. In one embodiment, the cancer is characterized by the lack of an EGFR mutation.

In one embodiment, the cancer is resistant to anti-PD1 therapy or anti-PD-L1 therapy. In one embodiment, the cancer has intrinsic resistance to anti-PD1 therapy or anti-PD-L1 therapy. In one embodiment, the cancer has acquired resistance to anti-PD1 therapy or anti-PD-L1 therapy. In one embodiment, the cancer is resistant to chemotherapy (e.g., platinum-containing chemotherapy). In one embodiment, the cancer has intrinsic resistance to chemotherapy (e.g., platinum-containing chemotherapy). In one embodiment, the cancer has acquired resistance to chemotherapy (e.g., platinum-containing chemotherapy).

In one embodiment, the cancer does not respond to or benefit from treatment with an immune checkpoint modulator when administered alone or as part of a treatment regimen that does not include an HDAC inhibitor.

In some embodiments, the patient is immunocompetent. In some embodiments, the patient has received an adoptive cell therapy. In some embodiments, the adoptive cell therapy is a TIL therapy or a CAR-T cell therapy.

Determining Whether a Subject Will Respond to Treatment with HDAC Inhibitors

In one embodiment, provided is a method for ascertaining susceptibility of a subject having or having been diagnosed with cancer to treatment with an HDAC inhibitor as described herein, the method comprising: determining: i) the presence or absence of a STK11 mutation; and/or ii) the level of STK11 activity or expression in the subject or a sample derived from the subject; wherein the presence of a STK11 mutation (e.g., an STK loss-of-function mutation) and/or a modified (e.g., decreased) level of STK11 activity or expression is indicative of susceptibility to treatment with an HDAC inhibitor.

In one embodiment, provided is a method for ascertaining susceptibility of a subject having or having been diagnosed with cancer to treatment with a combination of an HDAC inhibitor as described herein and an immune checkpoint modulator as described herein, the method comprising: determining: i) the presence or absence of a STK11 mutation; and/or ii) the level of STK11 activity or expression in the subject or a sample derived from the subject: wherein the presence of a STK11 mutation (e.g., an STK loss-of-function mutation) and/or a modified (e.g., decreased) level of STK11 activity or expression is indicative of susceptibility to treatment with a combination of an HDAC inhibitor and an immune checkpoint modulator.

In one embodiment, the level of STK11 expression is assessed by determining the copy number of the gene encoding STK11 relative to a control sample, wherein an increase in the copy number indicates an increased level of expression and a decrease in the copy number indicates a decreased level of expression. In one embodiment, the level of STK11 expression is assessed by determining the level of STK11 protein or mRNA relative to a control sample.

In one embodiment, provided is a method for ascertaining whether a subject having or having been diagnosed with cancer will respond to treatment with an HDAC inhibitor as described herein, the method comprising: determining the presence or absence of a STK11 mutation in the subject or a sample derived from the subject, wherein the presence of a STK11 mutation (e.g., an STK loss-of-function mutation) indicates that the subject will respond to treatment with an HDAC inhibitor.

In one embodiment, provided is a method for ascertaining whether a subject having or having been diagnosed with cancer will respond to treatment with an HDAC inhibitor as described herein and an immune checkpoint modulator as described herein, the method comprising: determining the presence or absence of a STK11 mutation in the subject or a sample derived from the subject, wherein the presence of a STK11 mutation (e.g., an STK loss-of-function mutation) indicates that the subject will respond to treatment with a combination of an HDAC inhibitor and an immune checkpoint modulator.

In one embodiment, provided is a method for ascertaining whether a subject having or having been diagnosed with cancer will respond to treatment with an HDAC inhibitor as described herein, the method comprising:
a) detecting levels of STK11 (e.g., STK11 protein and/or STK11 mRNA) in a cancer test sample (e.g., in a cancer sample obtained from the subject);
b) comparing the cancer test sample with a reference (e.g., a reference sample taken from a non-cancerous or normal control subject), wherein modified (e.g., decreased) levels of STK11 in said test sample indicates that the subject will respond to treatment with an HDAC inhibitor.

In one embodiment, provided is a method for ascertaining whether a subject having or having been diagnosed with cancer will respond to treatment with an HDAC inhibitor as described herein and an immune checkpoint modulator as described herein, the method comprising:
a) detecting levels of STK11 (e.g., STK11 protein and/or STK11 mRNA) in a cancer test sample (e.g., in a cancer sample obtained from the subject);
b) comparing the cancer test sample with a reference (e.g., a reference sample taken from a non-cancerous or normal control subject), wherein modified (e.g., decreased) levels of STK11 in said test sample indicates that the subject will respond to treatment with a combination of an HDAC inhibitor and an immune checkpoint modulator.

In one embodiment, provided is a method for ascertaining whether a subject having or having been diagnosed with cancer will respond to treatment with an HDAC inhibitor as described herein, the method comprising:
a) determining the copy number of the gene encoding STK11 in a cancer test sample (e.g., in a cancer sample obtained from the subject);
b) comparing the cancer test sample with a reference (e.g., a reference sample taken from a non-cancerous or normal control subject), wherein a decrease in the copy number indicates that the subject will respond to treatment with an HDAC inhibitor.

In one embodiment, provided is a method for ascertaining whether a subject having or having been diagnosed with cancer will respond to treatment with an HDAC inhibitor as described herein and an immune checkpoint modulator as described herein, the method comprising:
a) determining the copy number of the gene encoding STK11 in a cancer test sample (e.g., in a cancer sample obtained from the subject);
b) comparing the cancer test sample with a reference (e.g., a reference sample taken from a non-cancerous or normal control subject), wherein a decrease in the copy number indicates that the subject will respond to treatment with a combination of an HDAC inhibitor and an immune checkpoint modulator.

In one embodiment, the STK11 mutation is a loss of function mutation. In one embodiment, the modified level of STK11 activity or expression is determined by comparing the level of activity or expression in the subject or sample derived from the subject (e.g., a tumor sample) and comparing it with a control (e.g., a healthy subject or a sample derived from a healthy subject). In one embodiment, the modified level of STK11 activity is a reduced level of STK11 activity.

In one embodiment, the cancer is lung cancer (e.g., lung adenocarcinoma, non-small cell lung cancer (NSCLC), squamous cell lung carcinoma), colorectal cancer (e.g., colon adenocarcinoma, rectal adenocarcinoma), breast cancer (e.g., invasive ductal carcinoma), pancreatic cancer (e.g., pancreatic adenocarcinoma), endometrial cancer (e.g., endometrioid carcinoma), neuroendocrine cancer (e.g., large cell neuroendocrine carcinoma), melanoma, non-melanoma skin cancer (e.g., skin squamous cell carcinoma), cholangiocarcinoma, gallbladder cancer, ovarian cancer (e.g., ovarian serous adenocarcinoma), bladder cancer (e.g., bladder urothelial carcinoma), prostate cancer (e.g., prostate adenocarcinoma), cervical cancer, endocervical cancer or cancer of unknown primary (e.g., adenocarcinoma of unknown primary).

In one embodiment, the cancer is lung cancer (e.g., lung adenocarcinoma, non-small cell lung cancer (NSCLC)), colorectal cancer (e.g., colon adenocarcinoma), pancreatic cancer (e.g., pancreatic adenocarcinoma), endometrial cancer (e.g., endometrioid carcinoma), melanoma, non-melanoma skin cancer (e.g., skin squamous cell carcinoma), cholangiocarcinoma, ovarian cancer (e.g., ovarian serous adenocarcinoma), cervical cancer, endocervical cancer or cancer of unknown primary (e.g., adenocarcinoma of unknown primary).

In one embodiment, the cancer is lung cancer (e.g., lung adenocarcinoma, non-small cell lung cancer (NSCLC)), colorectal cancer (e.g., colon adenocarcinoma), pancreatic cancer (e.g., pancreatic adenocarcinoma), endometrial cancer (e.g., endometrioid carcinoma), melanoma, non-melanoma skin cancer (e.g., skin squamous cell carcinoma), cholangiocarcinoma, ovarian cancer (e.g., ovarian serous adenocarcinoma) or cancer of unknown primary (e.g., adenocarcinoma of unknown primary).

In one embodiment, the cancer is lung cancer (e.g., lung adenocarcinoma, non-small cell lung cancer (NSCLC)). In one embodiment, the cancer is lung adenocarcinoma. In one embodiment, the cancer is non-small cell lung cancer (NSCLC).

In some embodiments described herein, the cancer is colon cancer. In some embodiments described herein, the cancer is colon adenocarcinoma. In some embodiments described herein, the cancer is colorectal cancer.

Determining Whether a Cancer Will Respond to Treatment with HDAC Inhibitors

In one embodiment, provided is a method for ascertaining susceptibility of a cancer to treatment with an HDAC inhibitor as described herein, the method comprising: determining: i) the presence or absence of a STK11 mutation; and/or ii) the level of STK11 activity or expression in a cancer test sample (e.g., a sample derived from a subject); wherein the presence of a STK11 mutation (e.g., an STK loss-of-function mutation) and/or a modified (e.g., decreased) level of STK11 activity or expression is indicative of susceptibility to treatment with an HDAC inhibitor.

In one embodiment, provided is a method for ascertaining susceptibility of a cancer to treatment with a combination of an HDAC inhibitor as described herein and an immune checkpoint modulator as described herein, the method comprising: determining: i) the presence or absence of a STK11 mutation; and/or ii) the level of STK11 activity or expression in a cancer test sample (e.g., a sample derived from a subject); wherein the presence of a STK11 mutation (e.g., an STK loss-of-function mutation) and/or a modified (e.g., decreased) level of STK11 activity or expression is indicative of susceptibility to treatment with a combination of an HDAC inhibitor and an immune checkpoint modulator.

In one embodiment, the level of STK11 expression is assessed by determining the copy number of the gene encoding STK11 relative to a control sample, wherein an increase in the copy number indicates an increased level of expression and a decrease in the copy number indicates a decreased level of expression. In one embodiment, the level of STK11 expression is assessed by determining the level of STK11 protein or mRNA relative to a control sample.

In one embodiment, provided is a method for ascertaining whether a cancer will respond to treatment with an HDAC inhibitor as described herein, the method comprising: determining the presence or absence of a STK11 mutation in a cancer test sample (e.g., a sample derived from a subject), wherein the presence of a STK11 mutation (e.g., an STK loss-of-function mutation) indicates that the cancer will respond to treatment with an HDAC inhibitor.

In one embodiment, provided is a method for ascertaining whether a cancer will respond to treatment with an HDAC inhibitor as described herein and an immune checkpoint modulator as described herein, the method comprising: determining the presence or absence of a STK11 mutation in a cancer test sample (e.g., a sample derived from a subject), wherein the presence of a STK11 mutation (e.g., an STK loss-of-function mutation) indicates that the cancer will respond to treatment with a combination of an HDAC inhibitor and an immune checkpoint modulator.

In one embodiment, provided is a method for ascertaining whether a cancer will respond to treatment with an HDAC inhibitor as described herein, the method comprising:
a) detecting levels of STK11 (e.g., STK11 protein and/or STK11 mRNA) in a cancer test sample (e.g., in a cancer sample obtained from a subject);
b) comparing the cancer test sample with a reference (e.g., a reference sample taken from a non-cancerous or normal control subject), wherein modified (e.g., decreased) levels STK11 in said test sample indicates that the cancer will respond to treatment with an HDAC inhibitor.

In one embodiment, provided is a method for ascertaining whether a cancer will respond to treatment with an HDAC inhibitor as described herein and an immune checkpoint modulator as described herein, the method comprising:
a) detecting levels of STK11 (e.g., STK11 protein and/or STK11 mRNA) in a cancer test sample (e.g., in a cancer sample obtained from a subject);
b) comparing the cancer test sample with a reference (e.g., a reference sample taken from a non-cancerous or normal control subject), wherein modified (e.g., decreased) levels STK11 in said test sample indicates that the cancer will respond to treatment with a combination of an HDAC inhibitor and an immune checkpoint modulator.

In one embodiment, provided is a method for ascertaining whether a cancer will respond to treatment with an HDAC inhibitor as described herein, the method comprising:
a) determining the copy number of the gene encoding STK11 in a cancer test sample (e.g., in a cancer sample obtained from a subject);
b) comparing the cancer test sample with a reference (e.g., a reference sample taken from a non-cancerous or normal control subject), wherein a decrease in the copy number indicates that the cancer will respond to treatment with an HDAC inhibitor.

In one embodiment, provided is a method for ascertaining whether a cancer will respond to treatment with an HDAC inhibitor as described herein and an immune checkpoint modulator as described herein, the method comprising:
a) determining the copy number of the gene encoding STK11 in a cancer test sample (e.g., in a cancer sample obtained from a subject);
b) comparing the cancer test sample with a reference (e.g., a reference sample taken from a non-cancerous or normal control subject), wherein a decrease in the copy number indicates that the cancer will respond to treatment with a combination of an HDAC inhibitor and an immune checkpoint modulator.

In one embodiment, the STK11 mutation is a loss of function mutation. In one embodiment, the modified level of STK11 activity or expression is determined by comparing the level of activity or expression in a cancer test sample (e.g., a sample derived from a subject) and comparing it with a control (e.g., a sample derived from a healthy subject). In one embodiment, the modified level of STK11 activity is a reduced level of STK11 activity.

In one embodiment, the cancer is lung cancer (e.g., lung adenocarcinoma, non-small cell lung cancer (NSCLC), squamous cell lung carcinoma), colorectal cancer (e.g., colon adenocarcinoma, rectal adenocarcinoma), breast cancer (e.g., invasive ductal carcinoma), pancreatic cancer (e.g., pancreatic adenocarcinoma), endometrial cancer (e.g., endometrioid carcinoma), neuroendocrine cancer (e.g., large cell neuroendocrine carcinoma), melanoma, non-melanoma skin cancer (e.g., skin squamous cell carcinoma), cholangiocarcinoma, gallbladder cancer, ovarian cancer (e.g., ovarian serous adenocarcinoma), bladder cancer (e.g., bladder urothelial carcinoma), prostate cancer (e.g., prostate adenocarcinoma), cervical cancer, endocervical cancer or cancer of unknown primary (e.g., adenocarcinoma of unknown primary).

In one embodiment, the cancer is lung cancer (e.g., lung adenocarcinoma, non-small cell lung cancer (NSCLC)), colorectal cancer (e.g., colon adenocarcinoma), pancreatic cancer (e.g., pancreatic adenocarcinoma), endometrial cancer (e.g., endometrioid carcinoma), melanoma, non-melanoma skin cancer (e.g., skin squamous cell carcinoma), cholangiocarcinoma, ovarian cancer (e.g., ovarian serous adenocarcinoma), cervical cancer, endocervical cancer or cancer of unknown primary (e.g., adenocarcinoma of unknown primary).

In one embodiment, the cancer is lung cancer (e.g., lung adenocarcinoma, non-small cell lung cancer (NSCLC)), colorectal cancer (e.g., colon adenocarcinoma), pancreatic cancer (e.g., pancreatic adenocarcinoma), endometrial cancer (e.g., endometrioid carcinoma), melanoma, non-melanoma skin cancer (e.g., skin squamous cell carcinoma), cholangiocarcinoma, ovarian cancer (e.g., ovarian serous adenocarcinoma) or cancer of unknown primary (e.g., adenocarcinoma of unknown primary).

In one embodiment, the cancer is lung cancer (e.g., lung adenocarcinoma, non-small cell lung cancer (NSCLC)). In one embodiment, the cancer is lung adenocarcinoma. In one embodiment, the cancer is non-small cell lung cancer (NSCLC).

In some embodiments described herein, the cancer is colon cancer. In some embodiments described herein, the cancer is colon adenocarcinoma. In some embodiments described herein, the cancer is colorectal cancer.

### Sample Preparation

The present methods can comprise detecting somatic mutations, loss of heterozygosity, whole gene deletions, decreased expression, or DNA methylation in the promoter region of STK11. In some embodiments, mutations in STK11 may arise in a variety of sites in a cancer.

The disclosure further provides assays for the detection of levels of STK11, (e.g., STK11 protein and/or STK11 mRNA). In one embodiment, the disclosure provides assays for the detection of loss of STK11 protein expression (e.g., as measured by immunohistochemistry). The disclosure further provides assays for detecting STK11 mutations (e.g., STK11 loss of function mutations).

The gene mutation or expression may be analyzed from a patient sample. The patient sample can be any bodily tissue or fluid that includes nucleic acids from the cancer (e.g., lung cancer) in the subject. In some embodiments, the sample is a bodily fluid such as blood (e.g., serum or plasma) bone marrow, cerebral spinal fluid, peritoneal/pleural fluid, lymph fluid, ascites, serous fluid, sputum, lacrimal fluid, stool, and urine. In certain embodiments, the sample is a blood sample comprising circulating tumor cells or cell free DNA. In some embodiments, the sample is not a blood sample. In other embodiments, the sample can be a tissue, including normal or tumor tissue (e.g., a lung tissue). The tissue can be fresh frozen or formalin-fixed, paraffin-embedded (FFPE). In certain embodiments, a tumor FFPE (e.g., lung tumor FFPE) sample is obtained.

Methods and reagents are known in the art for obtaining, processing, and analyzing samples.

Cells can be harvested from a biological sample using standard techniques known in the art. Methods for extracting cellular DNA from fluid or tissue samples are known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed (e.g., with detergents) to extract DNA. After cell lysis, proteins are removed from DNA using various proteases. DNA is then extracted with phenol, precipitated in alcohol, and dissolved in an aqueous solution. Alternatively, genomic DNA can be extracted with kits such as the QIAamp® Tissue Kit (Qiagen, Chatsworth, Calif.) and the Wizard® Genomic DNA purification kit (Promega).

### Measurement of Gene Expression

In some embodiments, modified (e.g., reduced) levels of STK11 are modified (e.g., reduced) STK11 gene expression levels. In some embodiments, modified (e.g., reduced) levels of STK11 are modified (e.g., reduced) STK11 mRNA levels. Measurement of gene expression can be performed using any method or reagent known in the art.

Detection of gene expression can be by any appropriate method, including for example, detecting the quantity of mRNA transcribed from the gene or the quantity of cDNA produced from the reverse transcription of the mRNA transcribed from the gene or the quantity of the polypeptide or protein encoded by the gene. These methods can be performed on a sample-by-sample basis or modified for high throughput analysis. For example, using Affymetrix™ U133 microarray chips.

In some embodiments, gene expression is detected and quantitated by hybridization to a probe that specifically hybridizes to the appropriate probe for that biomarker. The probes also can be attached to a solid support for use in high throughput screening assays using methods known in the art.

In some embodiments, the expression level of a gene is determined through exposure of a nucleic acid sample to the probe-modified chip. Extracted nucleic acid is labeled, for example, with a fluorescent tag, preferably during an amplification step.

Hybridization of the labeled sample is performed at an appropriate stringency level. The degree of probe-nucleic acid hybridization is quantitatively measured using a detection device.

Alternatively, any one of gene copy number, transcription, or translation can be determined using known techniques. For example, an amplification method such as PCR may be useful. General procedures for PCR are taught in MacPherson et al., PCR: A Practical Approach, (IRL Press at Oxford University Press (1991)). However, PCR conditions used for each application reaction are empirically determined. A number of parameters influence the success of a reaction. Among them are annealing temperature and time, extension time, Mg 2+ and/or ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination. In some embodiments, the hybridized nucleic acids are detected by detecting one or more labels attached to the sample nucleic acids. The labels can be incorporated by any of a number of means well known to those of skill in the art. However, in some embodiments, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g., fluorescein-labeled UTP and/or CTP) incorporates a label into the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

In one example, the gene expression can be measured through an in-situ hybridization protocol that can detect RNA molecules on a slide containing tissue sections or cells (e.g., through RNAscope®).

Detectable labels suitable for use in the methods disclosed herein include any composition detectable by spectroscopic, photochemical, biochemical, immunochemical, electrical, optical or chemical means. Useful labels include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²P) enzymes (e.g., horseradish peroxidase, alkaline phosphatase and others commonly used in an ELISA), and calorimetric labels such as colloidal gold or colored glass or plastic (e.g., polystyrene, polypropylene, latex, etc.) beads.

Detection of labels is well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. The detectable label may be added to the target (sample) nucleic acid(s) prior to, or after the hybridization, such as described in WO 97/10365. These detectable labels are directly attached to or incorporated into the target (sample) nucleic acid prior to hybridization. In contrast, “indirect labels” are joined to the hybrid duplex after hybridization. Generally, the indirect label is attached to a binding moiety that has been attached to the target nucleic acid prior to the hybridization. For example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization with Nucleic Acid Probes, P. Tijssen, ed. Elsevier, N.Y. (1993).

In some embodiments, the detection of modified (e.g., reduced) levels of STK11 is by quantitative reverse transcriptase (RT)-polymerase chain reaction (PCR), RNA-Seq, or microarray.

### Detection of Polypeptides

Protein levels of STK11 can be determined by examining protein expression or the protein product. Determining the protein level involves measuring the amount of any immunospecific binding that occurs between an antibody that selectively recognizes and binds to the polypeptide of the biomarker in a sample obtained from a subject and comparing this to the amount of immunospecific binding of at least one biomarker in a control sample.

A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), Western blot analysis, immunoprecipitation assays, immunofluorescent assays, flow cytometry, immunohistochemistry, HPLC, mass spectrometry, confocal microscopy, enzymatic assays, surface plasmon resonance and PAGE-SDS.

In some embodiments, the detection of modified (e.g., reduced) STK11 protein levels is by Western blot. In some embodiments, the detection of modified (e.g., reduced) STK11 protein levels is by fluorescence-activated cell sorting (FACS). In some embodiments, the detection of modified (e.g., reduced) STK11 protein levels is by immunohistochemistry.

### Other Detection Methods

Mutations in targets of interest (e.g., STK11 mutations) can be detected by methods known to those of skill in the art.

For detection of somatic mutations, sequencing may be performed on DNA extracted from a tissue such as a tumor tissue. The tumor tissue can be fresh tissue or preserved tissue (e.g., formalin fixed tissue, e.g., paraffin-embedded tissue). Sequencing may also be performed using cell-free DNA. The coding regions and sometimes adjacent regions (e.g., introns, promoter) of genes of interest are sequenced using next generation sequencing (NGS) or Sanger sequencing.

Loss of function mutations or gene rearrangements may be detected or validated using secondary methods such as qPCR, PCR, immunohistochemistry, Sanger sequencing, comparative genomic hybridization, or the PacBio system.

### SELECTED EMBODIMENTS

Embodiment 1. A method of treating a subject having, or at risk of developing, a cancer, the method comprising administering to the subject an effective amount of a histone deacetylase (HDAC) inhibitor, wherein the cancer is identified as having modified STK11 activity or expression.

Embodiment 2. The method of embodiment 1, wherein the histone deacetylase inhibitor is administered in combination with one or more additional therapeutic agents.

Embodiment 3. The method of embodiment 2, wherein at least one of the additional therapeutic agents is an immune checkpoint modulator.

Embodiment 4. A method of treating a cancer in a subject comprising administering to the subject an immune checkpoint modulator and an HDAC inhibitor, wherein the treatment modulates and/or improves the Teff cell to Treg cell ratio in the tumor or tumor microenvironment, wherein the cancer is identified as having modified STK11 activity or expression.

Embodiment 5. A method of treating a cancer in a subject comprising administering to the subject an immune checkpoint modulator and an HDAC inhibitor, wherein the treatment reduces or depletes Treg cells in the tumor or tumor microenvironment, wherein the cancer is identified as having modified STK11 activity or expression.

Embodiment 6. A method of treating a cancer in a subject comprising administering to the subject an immune checkpoint modulator and an HDAC inhibitor, wherein the treatment induces or increases the expression of cytokines that promote anti-tumor activity, wherein the cancer is identified as having modified STK11 activity or expression.

Embodiment 7. The method of embodiment 6, wherein the cytokines are selected from the group of CXCL9, CXCL10, and CXCL 11.

Embodiment 8. A method of treating a cancer in a subject comprising administering to the subject an immune checkpoint modulator and an HDAC inhibitor, wherein the treatment reduces the expression of cytokines that promote Treg cell recruitment, wherein the cancer is identified as having modified STK11 activity or expression.

Embodiment 9. The method of embodiment 8, wherein the cytokines are CCL1 or CCL22.

Embodiment 10. A method of treating a cancer in a subject comprising administering to the subject an HDAC inhibitor, wherein the administering of the HDAC inhibitor does not reduce erythroid or myeloid cell viability, wherein the cancer is identified as having modified STK11 activity or expression.

Embodiment 11. A method of treating cancer in a subject, wherein the cancer presents an immune evasion phenotype characterized by STK11 mutant expression comprising: administering an HDAC1,2 selective inhibitor, wherein the HDAC1,2 selective inhibitor is capable of attenuating or reversing the immune evasion phenotype.

Embodiment 12. The method of embodiment 10 or 11, further comprising administering an immune checkpoint modulator.

Embodiment 13. A method of treating a cancer in a subject comprising administering to the subject an HDAC inhibitor, wherein an immune checkpoint modulator has been, is, or will be administered to the subject, wherein the cancer is identified as having modified STK11 activity or expression.

Embodiment 14. A method of treating a cancer in a subject comprising administering to the subject an immune checkpoint modulator, wherein an HDAC inhibitor has been, is, or will be administered to the subject, wherein the cancer is identified as having modified STK11 activity or expression.

Embodiment 15. The method of embodiment 2, wherein the HDAC inhibitor is administered in combination with two or more additional therapeutic agents, wherein at least two of the additional therapeutic agents are immune checkpoint modulators.

Embodiment 16. The method of any one of embodiments 3-15, wherein each immune checkpoint modulator is independently a checkpoint inhibitor, a T cell co-stimulatory receptor agonist or a dendritic cell co-stimulatory receptor agonist.

Embodiment 17. The method of any one of embodiments 3-16, wherein at least one immune checkpoint modulator is independently a T cell co-stimulatory receptor agonist or a dendritic cell co-stimulatory receptor agonist.

Embodiment 18. The method of any one of embodiments 3-16, wherein at least one immune checkpoint modulator is a checkpoint inhibitor.

Embodiment 19. The method of embodiment 18, wherein each checkpoint inhibitor is independently selected from an anti-CTLA-4 agent, an anti-PD-1 agent, an anti-PD-L1 agent, an anti-4-1BB agent, an anti-OX-40 agent, an anti-GITR agent, an anti-CD27 agent, an anti-CD28 agent, an anti-CD40 agent, an anti-LAG3 agent, an anti-ICOS agent, an anti-TWEAKR agent, an anti-HVEM agent, an anti-TIM-1 agent, an anti-TIM-3 agent, an anti-VISTA agent, and an anti-TIGIT agent.

Embodiment 20. The method of embodiment 18, wherein each checkpoint inhibitor is independently selected from an anti-CTLA-4 agent, an anti-PD-1 agent and an anti-PD-L1 agent.

Embodiment 21. The method of embodiment 18, wherein each checkpoint inhibitor is independently selected from an anti-PD1 agent and an anti-PD-L1 agent.

Embodiment 22. The method of embodiment 18, wherein the checkpoint inhibitor is an anti-PD1 agent.

Embodiment 23. The method of embodiment 18, wherein the checkpoint inhibitor is an anti-PD-L1 agent.

Embodiment 24. The method of embodiment 2, wherein the HDAC inhibitor is administered in combination with an anti-CTLA-4 agent and an anti PD-1 or anti PD-L1 agent.

Embodiment 25. The method of any one of embodiments 18-22, wherein each immune checkpoint inhibitor is independently an antibody.

Embodiment 26. The method of embodiment 25, wherein the each checkpoint inhibitor is independently selected from an anti-CTLA-4 antibody, an anti-PD-1 antibody, an anti-PD-L1 antibody, an anti-4-1 BB antibody, an anti-OX-40 antibody, an anti-GITR antibody, an anti-CD27 antibody, an anti-CD28 antibody, an anti-CD40 antibody, an anti-LAG3 antibody, an anti-ICOS antibody, an anti-TWEAKR antibody, an anti-HVEM antibody, an anti-TIM-1 antibody, an anti-TIM-3 antibody, an anti-VISTA antibody, and an anti-TIGIT antibody.

Embodiment 27. The method of embodiment 25, wherein each checkpoint inhibitor is independently selected from an anti-CTLA-4 antibody, an anti-PD-1 antibody and an anti-PD-L1 antibody.

Embodiment 28. The method of embodiment 25, wherein each immune checkpoint inhibitor is independently selected from nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab, BMS936559; durvalumab; avelumab; envafolimab; cosibelimab; sugemalimab, AUNP-12; atezolizumab and CA-170.

Embodiment 29. The method of embodiment 25, wherein each checkpoint inhibitor is independently selected from an anti-PD1 antibody and an anti-PD-L1 antibody.

Embodiment 30. The method of embodiment 25, wherein the checkpoint inhibitor is an anti-PD1 antibody.

Embodiment 31. The method of embodiment 25, wherein the checkpoint inhibitor is an anti-PD1-L1 antibody.

Embodiment 32. The method of embodiment 26 or 27, wherein the anti-CTLA-4 antibody is ipilimumab.

Embodiment 33. The method of any one of embodiments 26, 27, 29 and 30, wherein the anti-PD-1 antibody is pembrolizumab or nivolumab.

Embodiment 34. The method of any one of embodiments 26, 27, 29 and 30, wherein the anti-PD-1 antibody is pembrolizumab.

Embodiment 35. The method of any one of embodiments 26, 27, 29 and 30, wherein the anti-PD-1 antibody is nivolumab.

Embodiment 36. The method of any one of embodiments 26, 27, 29 and 31, wherein the anti-PD-L1 antibody is atezolizumab (CAS number 1380723-44-3), avelumab (CAS number 1537032-82-8), or durvalumab (CAS number 1428935-60-7).

Embodiment 37. The method of any one of embodiments 3-15, wherein the immune checkpoint modulator is a T cell co-stimulatory receptor agonist or a dendritic cell co-stimulatory receptor agonist.

Embodiment 38. The method of any one of embodiments 2-37, wherein at least one additional therapeutic agent is a targeted agent.

Embodiment 39. The method of embodiment 38, wherein each targeted agent is independently selected from an anti-angiogenesis agent (e.g., an anti-VEGF agent), a KRAS inhibitor, an ALK inhibitor, a ROS1 inhibitor, a BRAF inhibitor, a RET inhibitor, a MEK inhibitor, a MET inhibitor and a TRK inhibitor.

Embodiment 40. The method of embodiment 38, wherein each targeted agent is independently selected from bevacizumab, ramucirumab, sotorasib, crizotinib, ceritinib, alectinib, brigatinib, lorlatinib, entrectinib, dabrafenib, trametinib, capmatinib, tepotinib and larotrectinib.

Embodiment 41. The method of any one of embodiments 2-40, wherein at least one additional therapeutic agent is a chemotherapeutic agent.

Embodiment 42. The method of embodiment 41, wherein each chemotherapeutic agent is independently selected from cisplatin, carboplatin, paclitaxel, Albumin-bound paclitaxel (nab-paclitaxel), docetaxel, gemcitabine, vinorelbine, etoposide and pemetrexed.

Embodiment 43. The method of embodiment 41, wherein at least one chemotherapeutic agent is a platinum-containing therapeutic agent.

Embodiment 44. The method of embodiment 41, wherein one chemotherapeutic agent is a platinum-containing chemotherapeutic agent (e.g., cisplatin) and a second chemotherapeutic agent is pemetrexed.

Embodiment 45. The method of any one of embodiments 2-44, wherein at least one additional therapeutic agent is radiation.

Embodiment 46. The method of any one of embodiments 1-45, wherein the cancer is resistant to anti-PD1 therapy or anti-PD-L1 therapy.

Embodiment 47. The method of embodiment 46, wherein the cancer has intrinsic resistance to anti-PD1 therapy or anti-PD-L1 therapy.

Embodiment 48. The method of embodiment 46, wherein the cancer has acquired resistance to anti-PD1 therapy or anti-PD-L1 therapy.

Embodiment 49. The method of any one of embodiments 1-48, wherein the cancer is resistant to chemotherapy (e.g., platinum-containing chemotherapy).

Embodiment 50. The method of embodiment 49, wherein the cancer has intrinsic resistance to chemotherapy (e.g., platinum-containing chemotherapy).

Embodiment 51. The method of embodiment 49, wherein the cancer has acquired resistance to chemotherapy (e.g., platinum-containing chemotherapy).

Embodiment 52. The method of any one of embodiments 1-51, wherein the cancer does not respond to or benefit from treatment with an immune checkpoint modulator when administered alone or as part of a treatment regimen that does not include an HDAC inhibitor.

Embodiment 53. The method of any one of embodiments 1-51, wherein the cancer is selected from the group consisting of: lung cancer (e.g., lung adenocarcinoma, non-small cell lung cancer (NSCLC), squamous cell lung carcinoma), colorectal cancer (e.g., colon adenocarcinoma, rectal adenocarcinoma), breast cancer (e.g., invasive ductal carcinoma), pancreatic cancer (e.g., pancreatic adenocarcinoma), endometrial cancer (e.g., endometrioid carcinoma), neuroendocrine cancer (e.g., large cell neuroendocrine carcinoma), melanoma, non-melanoma skin cancer (e.g., skin squamous cell carcinoma), cholangiocarcinoma, gallbladder cancer, ovarian cancer (e.g., ovarian serous adenocarcinoma), bladder cancer (e.g., bladder urothelial carcinoma), prostate cancer (e.g., prostate adenocarcinoma), cervical cancer, endocervical cancer or cancer of unknown primary (e.g., adenocarcinoma of unknown primary).

Embodiment 54. The method of embodiment 52, wherein the cancer is lung cancer.

Embodiment 55. The method of embodiment 54, wherein the cancer is lung adenocarcinoma.

Embodiment 56. The method of embodiment 54, wherein the cancer is non-small cell lung cancer (NSCLC).

Embodiment 57. The method of embodiment 56, wherein the cancer is non-squamous non-small cell lung cancer (NSCLC).

Embodiment 58. The method of embodiment 52, wherein the cancer is colorectal cancer or colon adenocarcinoma.

Embodiment 59. The method of any one of embodiments 1-58, wherein the cancer is identified as having a STK11 mutation and one or more additional mutations.

Embodiment 60. The method of embodiment 59, wherein the additional mutations are selected from KRAS mutations and KEAP1 mutations.

Embodiment 61. The method of embodiment 59, wherein the additional mutations are KRAS mutations.

Embodiment 62. The method of embodiment 61, wherein the KRAS mutations are a mutations at position G12, optionally wherein the KRAS mutations are selected from G12D mutations, G12C mutations, G12V mutations or combinations thereof.

Embodiment 63. The method of embodiment 59, wherein the additional mutations are KEAP1 mutations.

Embodiment 64. The method of embodiment 59, wherein the additional mutations are KRAS mutations and KEAP1 mutations.

Embodiment 65. The method of any one of embodiments 1-64, wherein the cancer does not have an EGFR mutation.

Embodiment 66. The method of any one of embodiments 1-65, wherein the cancer has increased or decreased STK11 expression.

Embodiment 67. The method of embodiment 66, wherein the increased or decreased STK expression is assessed by determining copy number of the gene encoding STK11 relative to a control sample, wherein an increase in the copy number indicates an increased level of expression and a decrease in the copy number indicates a decreased level of expression.

Embodiment 68. The method of embodiment 66, wherein the increased or decreased STK expression is assessed by determining the level of STK11 protein or mRNA relative to a control sample.

Embodiment 69. The method of embodiment 67 or 68, wherein the cancer has decreased STK11 expression.

Embodiment 70. The method of any one of embodiments 1-65, wherein the cancer has a STK11 mutation.

Embodiment 71. The method of embodiment 70, wherein the STK11 mutation is a mutation selected from:
(i) a mutation in the nucleotide sequence encoding STK11;
(ii) a mutation in a regulatory sequence controlling expression of the nucleotide sequence encoding STK11;
(iii) a mutation in a nucleotide encoding a protein which interacts with the transcription product of the STK11 gene.

Embodiment 72. The method of embodiment 70, wherein the STK11 mutation is a mutation in the translation product of the STK11 gene.

Embodiment 73. The method of embodiment 70, wherein the STK11 mutation is a mutation in the transcription product of the STK11 gene.

Embodiment 74. The method of any one of embodiments 70-73, wherein the STK11 mutation is an inactivating (loss of function) mutation.

Embodiment 75. The method of any one of embodiments 66-69, wherein the increased or decreased STK11 expression is determined in a sample derived from the subject.

Embodiment 76. The method of embodiment 75, wherein the increased or decreased STK11 expression is determined relative to a control.

Embodiment 77. The method of embodiment 76, wherein the control is healthy tissue, preferably of the same tissue type as the cancer tissue.

Embodiment 78. The method of any one of embodiments 70-74, wherein the STK11 mutation is identified in a sample derived from the subject.

Embodiment 79. The method of embodiment 78, wherein the STK11 mutation is identified in a tumor sample derived from the subject.

Embodiment 80. The method of embodiment 78, wherein the STK11 mutation is not identified in a healthy tissue sample derived from the subject.

Embodiment 81. The method of any one of embodiments 1-80, wherein the STK11 mutation is not a germline mutation.

Embodiment 82. The method of any one of embodiments 2-81, wherein the HDAC inhibitor is administered concurrently with the additional therapeutic agents.

Embodiment 83. The method of any one of embodiments 2-81, wherein the HDAC inhibitor is administered separately from the additional therapeutic agents.

Embodiment 84. The method of any one of embodiments 2-81, wherein the HDAC inhibitor is administered sequentially from the additional therapeutic agents.

Embodiment 85. The method of any one of embodiments 2-81, wherein the HDAC inhibitor is administered prior to the additional therapeutic agents.

Embodiment 86. The method of any one of embodiments 2-81, wherein the HDAC inhibitor is administered subsequent to the additional therapeutic agents.

Embodiment 87. The method of any one of embodiments 2-81, comprising administering to the subject an immune checkpoint modulator followed by administering to the subject an HDAC inhibitor.

Embodiment 88. The method of any one of embodiments 2-81, comprising administering to the subject an HDAC inhibitor followed by administering to the subject an immune checkpoint modulator.

Embodiment 89. The method of any one of embodiments 2-81, comprising administering to the subject an immune checkpoint modulator, followed by administering to the subject a targeted agent, a chemotherapeutic agent, radiation or a combination thereof, followed by administering to the subject an HDAC inhibitor.

Embodiment 90. The method of any one of embodiments 2-81, comprising administering to the subject an immune checkpoint modulator, followed by administering to the subject an HDAC inhibitor, followed by administering to the subject a targeted agent, a chemotherapeutic agent, radiation or a combination thereof.

Embodiment 91. The method of any one of embodiments 2-81, comprising administering to the subject an HDAC inhibitor, followed by administering to the subject a targeted agent, a chemotherapeutic agent, radiation or a combination thereof, followed by administering to the subject an immune checkpoint modulator.

Embodiment 92. The method of any one of embodiments 2-81, comprising administering to the subject an HDAC inhibitor, followed by administering to the subject an immune checkpoint modulator, followed by administering to the subject a targeted agent, a chemotherapeutic agent, radiation or a combination thereof.

Embodiment 93. The method of any one of embodiments 2-81, comprising administering to the subject a targeted agent, a chemotherapeutic agent, radiation or a combination thereof, followed by administering to the subject an immune checkpoint modulator, followed by administering to the subject an HDAC inhibitor.

Embodiment 94. The method of any one of embodiments 2-81, comprising administering to the subject a targeted agent, a chemotherapeutic agent, radiation or a combination thereof, followed by administering to the subject an HDAC inhibitor, followed by administering to the subject an immune checkpoint modulator.

Embodiment 95. The method of any one of embodiments 1-94, wherein the subject has a cancer identified as having modified STK11 activity or expression.

Embodiment 96. The method of any one of embodiments 1-95, wherein the histone deacetylase inhibitor is a selective inhibitor of histone deacetylase 1 (HDAC1-selective inhibitor).

Embodiment 97. The method of any one of embodiments 1-95, wherein the histone deacetylase inhibitor is a selective inhibitor of histone deacetylase 1 and histone deacetylase 2 (HDAC1,2-selective inhibitor).

Embodiment 98. The method of any one of embodiments 1-95, wherein the histone deacetylase inhibitor is a selective HDAC Class I inhibitor.

Embodiment 99. The method of any one of embodiments 1-95, wherein the histone deacetylase inhibitor is a CoREST-selective deacetylase inhibitor.

Embodiment 100. A method of selecting a subject for treatment with an HDAC inhibitor the method comprising: identifying subjects having a cancer characterized by the presence of cells having modified STK11 activity or expression; and selecting thus identified subjects for treatment with an HDAC inhibitor.

Embodiment 101. A method of selecting a subject for treatment with a combination of an HDAC inhibitor and one or more additional therapeutic agents, the method comprising: identifying subjects having a cancer characterized by the presence of cells having modified STK11 activity or expression; and selecting thus the identified subjects for treatment with an HDAC inhibitor and one or more additional therapeutic agents.

Embodiment 102. The method of embodiment 101, wherein at least one of the additional therapeutic agents is an immune checkpoint modulator.

Embodiment 103. A method of selecting a subject for treatment with a combination of an HDAC inhibitor and two or more additional therapeutic agents, the method comprising: identifying subjects having a cancer characterized by the presence of cells having modified STK11 activity or expression; and selecting thus identified subjects for treatment with an HDAC inhibitor and two or more additional therapeutic agents, wherein at least two of the additional therapeutic agents are immune checkpoint modulators.

Embodiment 104. A method of selecting a subject for treatment with a combination of an HDAC inhibitor, an immune checkpoint modulator and one or more additional therapeutic agents selected from a chemotherapeutic agent, a targeted agent and radiotherapy, the method comprising: identifying subjects that have previously been treated with a combination of an immune checkpoint modulator and one or more additional therapeutic agents selected from a chemotherapeutic agent, a targeted agent and radiotherapy, wherein treatment with the combination of immune checkpoint modulator and additional therapeutic agent did not provide any additional benefit as compared to treatment with the additional therapeutic agent alone; and selecting thus identified subjects for treatment.

Embodiment 105. The method of embodiment 104, wherein the method further comprises: identifying subjects having a cancer characterized by the presence of cells having decreased STK11 activity or expression; and, selecting thus identified subjects for treatment.

Embodiment 106. The method of embodiment 102-105, wherein each immune checkpoint modulator is independently a checkpoint inhibitor, a T cell co-stimulatory receptor agonist or a dendritic cell co-stimulatory receptor agonist.

Embodiment 107. The method of any one of embodiments 102-106, wherein at least one immune checkpoint modulator is independently a T cell co-stimulatory receptor agonist or a dendritic cell co-stimulatory receptor agonist.

Embodiment 108. The method of any one of embodiments 102-107, wherein at least one immune checkpoint modulator is a checkpoint inhibitor.

Embodiment 109. The method of embodiment 108, wherein each checkpoint inhibitor is independently selected from an anti-CTLA-4 agent, an anti-PD-1 agent, an anti-PD-L1 agent, an anti-4-1 BB agent, an anti-OX-40 agent, an anti-GITR agent, an anti-CD27 agent, an anti-CD28 agent, an anti-CD40 agent, an anti-LAG3 agent, an anti-ICOS agent, an anti-TWEAKR agent, an anti-HVEM agent, an anti-TIM-1 agent, an anti-TIM-3 agent, an anti-VISTA agent, and an anti-TIGIT agent.

Embodiment 110. The method of embodiment 108, wherein each checkpoint inhibitor is independently selected from an anti-CTLA-4 agent, an anti-PD-1 agent and an anti-PD-L1 agent.

Embodiment 111. The method of embodiment 108, wherein each checkpoint inhibitor is independently selected from an anti-PD1 agent and an anti-PD-L1 agent.

Embodiment 112. The method of embodiment 108, wherein the checkpoint inhibitor is an anti-PD1 agent.

Embodiment 113. The method of embodiment 108, wherein the checkpoint inhibitor is an anti-PD-L1 agent.

Embodiment 114. The method of embodiment 103, wherein the HDAC inhibitor is administered in combination with an anti-CTLA-4 agent and an anti PD-1 or anti PD-L1 agent.

Embodiment 115. The method of any one of embodiments 108-114, wherein each immune checkpoint inhibitor is independently an antibody.

Embodiment 116. The method of embodiment 115, wherein the each checkpoint inhibitor is independently selected from an anti-CTLA-4 antibody, an anti-PD-1 antibody, an anti-PD-L1 antibody, an anti-4-1 BB antibody, an anti-OX-40 antibody, an anti-GITR antibody, an anti-CD27 antibody, an anti-CD28 antibody, an anti-CD40 antibody, an anti-LAG3 antibody, an anti-ICOS antibody, an anti-TWEAKR antibody, an anti-HVEM antibody, an anti-TIM-1 antibody, an anti-TIM-3 antibody, an anti-VISTA antibody, and an anti-TIGIT antibody.

Embodiment 117. The method of embodiment 115, wherein each checkpoint inhibitor is independently selected from an anti-CTLA-4 antibody, an anti-PD-1 antibody and an anti-PD-L1 antibody.

Embodiment 118. The method of embodiment 115, wherein each immune checkpoint inhibitor is independently selected from nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab and geptanolimab.

Embodiment 119. The method of embodiment 115, wherein each checkpoint inhibitor is independently selected from an anti-PD1 antibody and an anti-PD-L1 antibody.

Embodiment 120. The method of embodiment 115, wherein the checkpoint inhibitor is an anti-PD1 antibody.

Embodiment 121. The method of embodiment 115, wherein the checkpoint inhibitor is an anti-PD1-L1 antibody.

Embodiment 122. The method of embodiment 116 or 117, wherein the anti-CTLA-4 antibody is ipilimumab.

Embodiment 123. The method of any one of embodiments 116, 117, 119 and 120, wherein the anti-PD-1 antibody is pembrolizumab or nivolumab.

Embodiment 124. The method of any one of embodiments 116, 117, 119 and 120, wherein the anti-PD-1 antibody is pembrolizumab.

Embodiment 125. The method of any one of embodiments 116, 117, 119 and 120, wherein the anti-PD-1 antibody is nivolumab.

Embodiment 126. The method of any one of embodiments 116, 117, 119 and 121, wherein the anti-PD-L1 antibody is atezolizumab (CAS number 1380723-44-3), avelumab (CAS number 1537032-82-8), or durvalumab (CAS number 1428935-60-7).

Embodiment 127. The method of embodiment 102, wherein the immune checkpoint modulator is a T cell co-stimulatory receptor agonist or a dendritic cell co-stimulatory receptor agonist.

Embodiment 128. The method of any one of embodiments 101-127, wherein at least one additional therapeutic agent is targeted agent.

Embodiment 129. The method of embodiment 128, wherein each targeted agent is independently selected from an anti-angiogenesis agent (e.g., an anti-VEGF agent), a KRAS inhibitor, an ALK inhibitor, a ROS1 inhibitor, a BRAF inhibitor, a RET inhibitor, a MEK inhibitor, a MET inhibitor and a TRK inhibitor.

Embodiment 130. The method of embodiment 128, wherein each targeted agent is independently selected from bevacizumab, ramucirumab, sotorasib, crizotinib, ceritinib, alectinib, brigatinib, lorlatinib, entrectinib, dabrafenib, trametinib, capmatinib, tepotinib and larotrectinib.

Embodiment 131. The method of any one of embodiments 101-130, wherein at least one additional therapeutic agent is a chemotherapeutic agent.

Embodiment 132. The method of embodiment 131, wherein each chemotherapeutic agent is independently selected from cisplatin, carboplatin, paclitaxel, Albumin-bound paclitaxel (nab-paclitaxel), docetaxel, gemcitabine, vinorelbine, etoposide and pemetrexed.

Embodiment 133. The method of embodiment 131, wherein at least one chemotherapeutic agent is a platinum-containing therapeutic agent.

Embodiment 134. The method of embodiment 131, wherein one chemotherapeutic agent is a platinum-containing chemotherapeutic agent (e.g., cisplatin) and a second chemotherapeutic agent is pemetrexed.

Embodiment 135. The method of any one of embodiments 101-134, wherein at least one additional therapeutic agent is radiation.

Embodiment 136. The method of any one of embodiments 100-135, wherein the cancer is resistant to anti-PD1 therapy or anti-PD-L1 therapy.

Embodiment 137. The method of embodiment 136, wherein the cancer has intrinsic resistance to anti-PD1 therapy or anti-PD-L1 therapy.

Embodiment 138. The method of embodiment 136, wherein the cancer has acquired resistance to anti-PD1 therapy or anti-PD-L1 therapy.

Embodiment 139. The method of any one of embodiments 100-138, wherein the cancer is resistant to chemotherapy (e.g., platinum-containing chemotherapy).

Embodiment 140. The method of embodiment 139, wherein the cancer has intrinsic resistance to chemotherapy (e.g., platinum-containing chemotherapy).

Embodiment 141. The method of embodiment 140, wherein the cancer has acquired resistance to chemotherapy (e.g., platinum-containing chemotherapy).

Embodiment 142. The method of any one of embodiments 100-141, wherein the cancer does not respond to or benefit from treatment with an immune checkpoint modulator when administered alone or as part of a treatment regimen that does not include an HDAC inhibitor.

Embodiment 143. The method of any one of embodiments 100-142, wherein the cancer is selected from the group consisting of: lung cancer (e.g., lung adenocarcinoma, non-small cell lung cancer (NSCLC), squamous cell lung carcinoma), colorectal cancer (e.g., colon adenocarcinoma, rectal adenocarcinoma), breast cancer (e.g., invasive ductal carcinoma), pancreatic cancer (e.g., pancreatic adenocarcinoma), endometrial cancer (e.g., endometrioid carcinoma), neuroendocrine cancer (e.g., large cell neuroendocrine carcinoma), melanoma, non-melanoma skin cancer (e.g., skin squamous cell carcinoma), cholangiocarcinoma, gallbladder cancer, ovarian cancer (e.g., ovarian serous adenocarcinoma), bladder cancer (e.g., bladder urothelial carcinoma), prostate cancer (e.g., prostate adenocarcinoma), cervical cancer, endocervical cancer or cancer of unknown primary (e.g., adenocarcinoma of unknown primary).

Embodiment 144. The method of embodiment 142, wherein the cancer is lung cancer.

Embodiment 145. The method of embodiment 144, wherein the cancer is lung adenocarcinoma.

Embodiment 146. The method of embodiment 144, wherein the cancer is non-small cell lung cancer (NSCLC).

Embodiment 147. The method of embodiment 146, wherein the cancer is non-squamous non-small cell lung cancer (NSCLC).

Embodiment 148. The method of any one of embodiments 100-147, wherein the cancer is identified as having a STK11 mutation and one or more additional mutations.

Embodiment 149. The method of embodiment 148, wherein the additional mutations are selected from KRAS mutations and KEAP1 mutations.

Embodiment 150. The method of embodiment 148, wherein the additional mutations are KRAS mutations.

Embodiment 151. The method of embodiment 150, wherein the KRAS mutations are a mutations at position G12, optionally wherein the KRAS mutations are selected from G12D mutations, G12C mutations, G12V mutations or combinations thereof.

Embodiment 152. The method of embodiment 148, wherein the additional mutations are KEAP1 mutations.

Embodiment 153. The method of embodiment 148, wherein the additional mutations are KRAS mutations and KEAP1 mutations.

Embodiment 154. The method of any one of embodiments 100-153, wherein the cancer does not have an EGFR mutation.

Embodiment 155. The method of any one of embodiments 100-154, wherein the cancer has increased or decreased STK11 expression.

Embodiment 156. The method of embodiment 155, wherein the increased or decreased STK expression is assessed by determining copy number of the gene encoding STK11 relative to a control sample, wherein an increase in the copy number indicates an increased level of expression and a decrease in the copy number indicates a decreased level of expression.

Embodiment 157. The method of embodiment 155, wherein the increased or decreased STK expression is assessed by determining the level of STK11 protein or mRNA relative to a control sample.

Embodiment 158. The method of embodiment 155 to 157, wherein the cancer has decreased STK11 expression.

Embodiment 159. The method of any one of embodiments 100-154, wherein the cancer has a STK11 mutation.

Embodiment 160. The method of embodiment 159, wherein the STK11 mutation is a mutation selected from:
(i) a mutation in the nucleotide sequence encoding STK11;
(ii) a mutation in a regulatory sequence controlling expression of the nucleotide sequence encoding STK11;
(iii) a mutation in a nucleotide encoding a protein which interacts with the transcription product of the STK11 gene.

Embodiment 161. The method of embodiment 159, wherein the STK11 mutation is a mutation in the translation product of the STK11 gene.

Embodiment 162. The method of embodiment 159, wherein the STK11 mutation is a mutation in the transcription product of the STK11 gene.

Embodiment 163. The method of any one of embodiments 159-161, wherein the STK11 mutation is an inactivating (loss of function) mutation.

Embodiment 164. The method of any one of embodiments 155-158, wherein the increased or decreased STK11 expression is determined in a sample derived from the subject.

Embodiment 165. The method of embodiment 164, wherein the increased or decreased STK11 expression is determined relative to a control.

Embodiment 166. The method of embodiment 165, wherein the control is healthy tissue, preferably of the same tissue type as the cancer tissue.

Embodiment 167. The method of any one of embodiments 159-163, wherein the STK11 mutation is identified in a sample derived from the subject.

Embodiment 168. The method of embodiment 167, wherein the STK11 mutation is identified in a tumor sample derived from the subject.

Embodiment 169. The method of embodiment 167, wherein the STK11 mutation is not identified in a healthy tissue sample derived from the subject.

Embodiment 170. The method of any one of embodiments 100-169, wherein the STK11 mutation is not a germline mutation.

Embodiment 171. A method of treating a cancer in a subject comprising administering to the subject a combination of an HDAC inhibitor and an immune checkpoint modulator, wherein the subject has been selected for treatment using a method according to any one of embodiments 100-170.

Embodiment 172. A method of treating a cancer in a subject comprising administering to the subject an HDAC inhibitor, wherein an immune checkpoint modulator has been, is, or will be administered to the subject, wherein the subject has been selected for treatment using a method according to any one of embodiments 100-170.

Embodiment 173. A method of treating a cancer in a subject comprising administering to the subject an immune checkpoint modulator, wherein an HDAC inhibitor has been, is, or will be administered to the subject, wherein the subject has been selected for treatment using a method according to any one of embodiments 100-170.

Embodiment 174. The method of any one of embodiments 100-173 wherein the histone deacetylase inhibitor is a selective inhibitor of histone deacetylase 1 (HDAC1-selective inhibitor).

Embodiment 175. The method of any one of embodiments 100-173 wherein the histone deacetylase inhibitor is a selective inhibitor of histone deacetylase 1 and histone deacetylase 2 (HDAC1,2-selective inhibitor).

Embodiment 176. The method of any one of embodiments 100-173 wherein the histone deacetylase inhibitor is a selective HDAC Class I inhibitor.

Embodiment 177. The method of any one of embodiments 100-173 wherein the histone deacetylase inhibitor is a CoREST-selective deacetylase inhibitor.

Embodiment 178. An HDAC inhibitor for use in a method of treating a subject having, or at risk of developing, a cancer, the method comprising administering to the subject an effective amount of a histone deacetylase (HDAC) inhibitor, wherein the cancer is identified as having modified STK11 activity or expression.

Embodiment 179. The HDAC inhibitor for use of embodiment 178, wherein the histone deacetylase inhibitor is administered in combination with one or more additional therapeutic agents.

Embodiment 180. The HDAC inhibitor for use of embodiment 179, wherein at least one of the additional therapeutic agents is an immune checkpoint modulator.

Embodiment 181. An HDAC inhibitor for use in a method of treating a cancer in a subject comprising administering to the subject an immune checkpoint modulator and an HDAC inhibitor, wherein the treatment modulates and/or improves the Teff cell to Treg cell ratio in the tumor or tumor microenvironment, wherein the cancer is identified as having modified STK11 activity or expression.

Embodiment 182. An HDAC inhibitor for use in a method of treating a cancer in a subject comprising administering to the subject an immune checkpoint modulator and an HDAC inhibitor, wherein the treatment reduces or depletes Treg cells in the tumor or tumor microenvironment, wherein the cancer is identified as having modified STK11 activity or expression.

Embodiment 183. An HDAC inhibitor for use in a method of treating a cancer in a subject comprising administering to the subject an immune checkpoint modulator and an HDAC inhibitor, wherein the treatment induces or increases the expression of cytokines that promote anti-tumor activity, wherein the cancer is identified as having modified STK11 activity or expression.

Embodiment 184. The HDAC inhibitor for use in a method of embodiment 183, wherein the cytokines are selected from the group of CXCL9, CXCL10, and CXCL 11.

Embodiment 185. An HDAC inhibitor for use in a method of treating a cancer in a subject comprising administering to the subject an immune checkpoint modulator and an HDAC inhibitor, wherein the treatment reduces the expression of cytokines that promote Treg cell recruitment, wherein the cancer is identified as having modified STK11 activity or expression.

Embodiment 186. The HDAC inhibitor for use in the method of embodiment 185, wherein the cytokines are CCL1 or CCL22.

Embodiment 187. An HDAC inhibitor for use in a method of treating a cancer in a subject comprising administering to the subject an HDAC inhibitor, wherein the administering of the HDAC inhibitor does not reduce erythroid or myeloid cell viability, wherein the cancer is identified as having modified STK11 activity or expression.

Embodiment 188. An HDAC inhibitor for use in a method of treating cancer in a subject, wherein the cancer presents an immune evasion phenotype characterized by STK11 mutant expression comprising: administering an HDAC1,2 selective inhibitor, wherein the HDAC1,2 selective inhibitor is capable of attenuating or reversing the immune evasion phenotype.

Embodiment 189. The HDAC inhibitor for use in a method of embodiment 187 or 188, further comprising administering an immune checkpoint modulator.

Embodiment 190. An HDAC inhibitor for use in a method of treating a cancer in a subject comprising administering to the subject an HDAC inhibitor, wherein an immune checkpoint modulator has been, is, or will be administered to the subject, wherein the cancer is identified as having modified STK11 activity or expression.

Embodiment 191. An HDAC inhibitor for use in a method of treating a cancer in a subject comprising administering to the subject an immune checkpoint modulator, wherein an HDAC inhibitor has been, is, or will be administered to the subject, wherein the cancer is identified as having modified STK11 activity or expression.

Embodiment 192. The HDAC inhibitor for use of embodiment 179, wherein the HDAC inhibitor is administered in combination with two or more additional therapeutic agents, wherein at least two of the additional therapeutic agents are immune checkpoint modulators.

Embodiment 193. The HDAC inhibitor for use of any one of embodiments 180-192, wherein each immune checkpoint modulator is independently a checkpoint inhibitor, a T cell co-stimulatory receptor agonist or a dendritic cell co-stimulatory receptor agonist.

Embodiment 194. The HDAC inhibitor for use of any one of embodiments 180-193, wherein at least one immune checkpoint modulator is independently a T cell co-stimulatory receptor agonist or a dendritic cell co-stimulatory receptor agonist.

Embodiment 195. The HDAC inhibitor for use of any one of embodiments 180-193, wherein at least one immune checkpoint modulator is a checkpoint inhibitor.

Embodiment 196. The HDAC inhibitor for use of embodiment 195, wherein each checkpoint inhibitor is independently selected from an anti-CTLA-4 agent, an anti-PD-1 agent, an anti-PD-L1 agent, an anti-4-1 BB agent, an anti-OX-40 agent, an anti-GITR agent, an anti-CD27 agent, an anti-CD28 agent, an anti-CD40 agent, an anti-LAG3 agent, an anti-ICOS agent, an anti-TWEAKR agent, an anti-HVEM agent, an anti-TIM-1 agent, an anti-TIM-3 agent, an anti-VISTA agent, and an anti-TIGIT agent.

Embodiment 197. The HDAC inhibitor for use of embodiment 195, wherein each checkpoint inhibitor is independently selected from an anti-CTLA-4 agent, an anti-PD-1 agent and an anti-PD-L1 agent.

Embodiment 198. The HDAC inhibitor for use of embodiment 195, wherein each checkpoint inhibitor is independently selected from an anti-PD1 agent and an anti-PD-L1 agent.

Embodiment 199. The HDAC inhibitor for use of embodiment 195, wherein the checkpoint inhibitor is an anti-PD1 agent.

Embodiment 200. The HDAC inhibitor for use of embodiment 195, wherein the checkpoint inhibitor is an anti-PD-L1 agent.

Embodiment 201. The HDAC inhibitor for use of embodiment 179, wherein the HDAC inhibitor is administered in combination with an anti-CTLA-4 agent and an anti PD-1 or anti PD-L1 agent.

Embodiment 202. The HDAC inhibitor for use of any one of embodiments 195-200, wherein each immune checkpoint inhibitor is independently an antibody.

Embodiment 203. The HDAC inhibitor for use of embodiment 202, wherein the each checkpoint inhibitor is independently selected from an anti-CTLA-4 antibody, an anti-PD-1 antibody, an anti-PD-L1 antibody, an anti-4-1 BB antibody, an anti-OX-40 antibody, an anti-GITR antibody, an anti-CD27 antibody, an anti-CD28 antibody, an anti-CD40 antibody, an anti-LAG3 antibody, an anti-ICOS antibody, an anti-TWEAKR antibody, an anti-HVEM antibody, an anti-TIM-1 antibody, an anti-TIM-3 antibody, an anti-VISTA antibody, and an anti-TIGIT antibody.

Embodiment 204. The HDAC inhibitor for use of embodiment 202, wherein each checkpoint inhibitor is independently selected from an anti-CTLA-4 antibody, an anti-PD-1 antibody and an anti-PD-L1 antibody.

Embodiment 205. The HDAC inhibitor for use of embodiment 202, wherein each immune checkpoint inhibitor is independently selected from nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab and geptanolimab.

Embodiment 206. The HDAC inhibitor for use of embodiment 202, wherein each checkpoint inhibitor is independently selected from an anti-PD1 antibody and an anti-PD-L1 antibody.

Embodiment 207. The HDAC inhibitor for use of embodiment 202, wherein the checkpoint inhibitor is an anti-PD1 antibody.

Embodiment 208. The HDAC inhibitor for use of embodiment 202, wherein the checkpoint inhibitor is an anti-PD1-L1 antibody.

Embodiment 209. The HDAC inhibitor for use of embodiment 203 or 204, wherein the anti-CTLA-4 antibody is ipilimumab.

Embodiment 210. The HDAC inhibitor for use of any one of embodiments 203, 204, 206 and 207, wherein the anti-PD-1 antibody is pembrolizumab or nivolumab.

Embodiment 211. The HDAC inhibitor for use of any one of embodiments 203, 204, 206 and 207, wherein the anti-PD-1 antibody is pembrolizumab.

Embodiment 212. The HDAC inhibitor for use of any one of embodiments 203, 204, 206 and 207, wherein the anti-PD-1 antibody is nivolumab.

Embodiment 213. The HDAC inhibitor for use of any one of embodiments 203, 204, 206 and 208, wherein the anti-PD-L1 antibody is atezolizumab (CAS number 1380723-44-3), avelumab (CAS number 1537032-82-8), or durvalumab (CAS number 1428935-60-7).

Embodiment 214. The HDAC inhibitor for use of any one of embodiments 180-193, wherein the immune checkpoint modulator is a T cell co-stimulatory receptor agonist or a dendritic cell co-stimulatory receptor agonist.

Embodiment 215. The HDAC inhibitor for use of any one of embodiments 179-214, wherein at least one additional therapeutic agent is targeted agent.

Embodiment 216. The HDAC inhibitor for use of embodiment 215, wherein each targeted agent is independently selected from an anti-angiogenesis agent (e.g., an anti-VEGF agent), a KRAS inhibitor, an ALK inhibitor, a ROS1 inhibitor, a BRAF inhibitor, a RET inhibitor, a MEK inhibitor, a MET inhibitor and a TRK inhibitor.

Embodiment 217. The HDAC inhibitor for use of embodiment 215, wherein each targeted agent is independently selected from bevacizumab, ramucirumab, sotorasib, crizotinib, ceritinib, alectinib, brigatinib, lorlatinib, entrectinib, dabrafenib, trametinib, capmatinib, tepotinib and larotrectinib.

Embodiment 218. The HDAC inhibitor for use of any one of embodiments 179-217, wherein at least one additional therapeutic agent is a chemotherapeutic agent.

Embodiment 219. The HDAC inhibitor for use of embodiment 218, wherein each chemotherapeutic agent is independently selected from cisplatin, carboplatin, paclitaxel, Albumin-bound paclitaxel (nab-paclitaxel), docetaxel, gemcitabine, vinorelbine, etoposide and pemetrexed.

Embodiment 220. The HDAC inhibitor for use of embodiment 218, wherein at least one chemotherapeutic agent is a platinum-containing therapeutic agent.

Embodiment 221. The HDAC inhibitor for use of embodiment 218, wherein one chemotherapeutic agent is a platinum-containing chemotherapeutic agent (e.g., cisplatin) and a second chemotherapeutic agent is pemetrexed.

Embodiment 222. The HDAC inhibitor for use of any one of embodiments 179-221, wherein at least one additional therapeutic agent is radiation.

Embodiment 223. The HDAC inhibitor for use of any one of embodiments 178-222, wherein the cancer is resistant to anti-PD1 therapy or anti-PD-L1 therapy.

Embodiment 224. The HDAC inhibitor for use of embodiment 223, wherein the cancer has intrinsic resistance to anti-PD1 therapy or anti-PD-L1 therapy.

Embodiment 225. The HDAC inhibitor for use of embodiment 223, wherein the cancer has acquired resistance to anti-PD1 therapy or anti-PD-L1 therapy.

Embodiment 226. The HDAC inhibitor for use of any one of embodiments 178-225, wherein the cancer is resistant to chemotherapy (e.g., platinum-containing chemotherapy).

Embodiment 227. The HDAC inhibitor for use of embodiment 226, wherein the cancer has intrinsic resistance to chemotherapy (e.g., platinum-containing chemotherapy).

Embodiment 228. The HDAC inhibitor for use of embodiment 226, wherein the cancer has acquired resistance to chemotherapy (e.g., platinum-containing chemotherapy).

Embodiment 229. The HDAC inhibitor for use of any one of embodiments 178-228, wherein the cancer does not respond to or benefit from treatment with an immune checkpoint modulator when administered alone or as part of a treatment regimen that does not include an HDAC inhibitor.

Embodiment 230. The HDAC inhibitor for use of any one of embodiments 178-229, wherein the cancer is selected from the group consisting of: lung cancer (e.g., lung adenocarcinoma, non-small cell lung cancer (NSCLC), squamous cell lung carcinoma), colorectal cancer (e.g., colon adenocarcinoma, rectal adenocarcinoma), breast cancer (e.g., invasive ductal carcinoma), pancreatic cancer (e.g., pancreatic adenocarcinoma), endometrial cancer (e.g., endometrioid carcinoma), neuroendocrine cancer (e.g., large cell neuroendocrine carcinoma), melanoma, non-melanoma skin cancer (e.g., skin squamous cell carcinoma), cholangiocarcinoma, gallbladder cancer, ovarian cancer (e.g., ovarian serous adenocarcinoma), bladder cancer (e.g., bladder urothelial carcinoma), prostate cancer (e.g., prostate adenocarcinoma), cervical cancer, endocervical cancer or cancer of unknown primary (e.g., adenocarcinoma of unknown primary).

Embodiment 231. The HDAC inhibitor for use of embodiment 230, wherein the cancer is lung cancer.

Embodiment 232. The HDAC inhibitor for use of embodiment 231, wherein the cancer is lung adenocarcinoma.

Embodiment 233. The HDAC inhibitor for use of embodiment 231, wherein the cancer is non-small cell lung cancer (NSCLC).

Embodiment 234. The HDAC inhibitor for use of embodiment 233, wherein the cancer is non-squamous non-small cell lung cancer (NSCLC).

Embodiment 235. The HDAC inhibitor for use of embodiment 230, wherein the cancer is colorectal cancer or colon adenocarcinoma.

Embodiment 236. The HDAC inhibitor for use of any one of embodiments 178-235, wherein the cancer is identified as having a STK11 mutation and one or more additional mutations.

Embodiment 237. The HDAC inhibitor for use of embodiment 236, wherein the additional mutations are selected from KRAS mutations and KEAP1 mutations.

Embodiment 238. The HDAC inhibitor for use of embodiment 236, wherein the additional mutations are KRAS mutations.

Embodiment 239. The HDAC inhibitor for use of embodiment 238, wherein the KRAS mutations are a mutations at position G12, optionally wherein the KRAS mutations are selected from G12D mutations, G12C mutations, G12V mutations or combinations thereof.

Embodiment 240. The HDAC inhibitor for use of embodiment 236, wherein the additional mutations are KEAP1 mutations.

Embodiment 241. The HDAC inhibitor for use of embodiment 236, wherein the additional mutations are KRAS mutations and KEAP1 mutations.

Embodiment 242. The HDAC inhibitor for use of any one of embodiments 178-241, wherein the cancer does not have an EGFR mutation.

Embodiment 243. The HDAC inhibitor for use of any one of embodiments 178-242, wherein the cancer has increased or decreased STK11 expression.

Embodiment 244. The HDAC inhibitor for use of embodiment 243, wherein the increased or decreased STK expression is assessed by determining copy number of the gene encoding STK11 relative to a control sample, wherein an increase in the copy number indicates an increased level of expression and a decrease in the copy number indicates a decreased level of expression.

Embodiment 245. The HDAC inhibitor for use of embodiment 243, wherein the increased or decreased STK expression is assessed by determining the level of STK11 protein or mRNA relative to a control sample.

Embodiment 246. The HDAC inhibitor for use of embodiment 244 or 245, wherein the cancer has decreased STK11 expression.

Embodiment 247. The HDAC inhibitor for use of any one of embodiments 178-242, wherein the cancer has a STK11 mutation.

Embodiment 248. The HDAC inhibitor for use of embodiment 247, wherein the STK11 mutation is a mutation selected from:
(i) a mutation in the nucleotide sequence encoding STK11;
(ii) a mutation in a regulatory sequence controlling expression of the nucleotide sequence encoding STK11;
(iii) a mutation in a nucleotide encoding a protein which interacts with the transcription product of the STK11 gene.

Embodiment 249. The HDAC inhibitor for use of embodiment 247, wherein the STK11 mutation is a mutation in the translation product of the STK11 gene.

Embodiment 250. The HDAC inhibitor for use of embodiment 247, wherein the STK11 mutation is a mutation in the transcription product of the STK11 gene.

Embodiment 251. The HDAC inhibitor for use of any one of embodiments 247-250, wherein the STK11 mutation is an inactivating (loss of function) mutation.

Embodiment 252. The HDAC inhibitor for use of any one of embodiments 243-246, wherein the increased or decreased STK11 expression is determined in a sample derived from the subject.

Embodiment 253. The HDAC inhibitor for use of embodiment 252, wherein the increased or decreased STK11 expression is determined relative to a control.

Embodiment 254. The HDAC inhibitor for use of embodiment 253, wherein the control is healthy tissue, preferably of the same tissue type as the cancer tissue.

Embodiment 255. The HDAC inhibitor for use of any one of embodiments 247-250, wherein the STK11 mutation is identified in a sample derived from the subject.

Embodiment 256. The HDAC inhibitor for use of embodiment 255, wherein the STK11 mutation is identified in a tumor sample derived from the subject.

Embodiment 257. The HDAC inhibitor for use of embodiment 255, wherein the STK11 mutation is not identified in a healthy tissue sample derived from the subject.

Embodiment 258. The HDAC inhibitor for use of any one of embodiments 178-257, wherein the STK11 mutation is not a germline mutation.

Embodiment 259. The HDAC inhibitor for use of any one of embodiments 179-258, wherein the HDAC inhibitor is administered concurrently with the additional therapeutic agents.

Embodiment 260. The HDAC inhibitor for use of any one of embodiments 179-258, wherein the HDAC inhibitor is administered separately from the additional therapeutic agents.

Embodiment 261. The HDAC inhibitor for use of any one of embodiments 179-258, wherein the HDAC inhibitor is administered sequentially from the additional therapeutic agents.

Embodiment 262. The HDAC inhibitor for use of any one of embodiments 179-258, wherein the HDAC inhibitor is administered prior to the additional therapeutic agents.

Embodiment 263. The HDAC inhibitor for use of any one of embodiments 179-258, wherein the HDAC inhibitor is administered subsequent to the additional therapeutic agents.

Embodiment 264. The HDAC inhibitor for use of any one of embodiments 179-258, wherein the method comprises administering to the subject an immune checkpoint modulator followed by administering to the subject an HDAC inhibitor.

Embodiment 265. The HDAC inhibitor for use of any one of embodiments 179-258, wherein the method comprises administering to the subject an HDAC inhibitor followed by administering to the subject an immune checkpoint modulator.

Embodiment 266. The HDAC inhibitor for use of any one of embodiments 179-258, wherein the method comprises administering to the subject an immune checkpoint modulator, followed by administering to the subject a targeted agent, a chemotherapeutic agent, radiation or a combination thereof, followed by administering to the subject an HDAC inhibitor.

Embodiment 267. The HDAC inhibitor for use of any one of embodiments 179-258, wherein the method comprises administering to the subject an immune checkpoint modulator, followed by administering to the subject an HDAC inhibitor, followed by administering to the subject a targeted agent, a chemotherapeutic agent, radiation or a combination thereof.

Embodiment 268. The HDAC inhibitor for use of any one of embodiments 179-258, wherein the method comprises administering to the subject an HDAC inhibitor, followed by administering to the subject a targeted agent, a chemotherapeutic agent, radiation or a combination thereof, followed by administering to the subject an immune checkpoint modulator.

Embodiment 269. The HDAC inhibitor for use of any one of embodiments 179-258, wherein the method comprises administering to the subject an HDAC inhibitor, followed by administering to the subject an immune checkpoint modulator, followed by administering to the subject a targeted agent, a chemotherapeutic agent, radiation or a combination thereof.

Embodiment 270. The HDAC inhibitor for use of any one of embodiments 179-258, wherein the method comprises administering to the subject a targeted agent, a chemotherapeutic agent, radiation or a combination thereof, followed by administering to the subject an immune checkpoint modulator, followed by administering to the subject an HDAC inhibitor.

Embodiment 271. The HDAC inhibitor for use of any one of embodiments 179-258, wherein the method comprises administering to the subject a targeted agent, a chemotherapeutic agent, radiation or a combination thereof, followed by administering to the subject an HDAC inhibitor, followed by administering to the subject an immune checkpoint modulator.

Embodiment 272. The HDAC inhibitor for use of any one of embodiments 178-271, wherein the subject has a cancer identified as having modified STK11 activity or expression.

Embodiment 273. The HDAC inhibitor for use of any one of embodiments 178-272, wherein the histone deacetylase inhibitor is a selective inhibitor of histone deacetylase 1 (HDAC1-selective inhibitor).

Embodiment 274. The HDAC inhibitor for use of any one of embodiments 178-272, wherein the histone deacetylase inhibitor is a selective inhibitor of histone deacetylase 1 and histone deacetylase 2 (HDAC1,2-selective inhibitor).

Embodiment 275. The HDAC inhibitor for use of any one of embodiments 178-272, wherein the histone deacetylase inhibitor is a selective HDAC Class I inhibitor.

Embodiment 276. The HDAC inhibitor for use of any one of embodiments 178-272, wherein the histone deacetylase inhibitor is a CoREST-selective deacetylase inhibitor.

Embodiment 277. Use of an HDAC inhibitor in the manufacturing of a medicament for treating a subject having, or at risk of developing, a cancer, wherein the treatment comprises administering to the subject an effective amount of the histone deacetylase (HDAC) inhibitor, wherein the cancer is identified as having modified STK11 activity or expression.

Embodiment 278. The use of embodiment 277, wherein the histone deacetylase inhibitor is administered in combination with one or more additional therapeutic agents.

Embodiment 279. The use of embodiment 278, wherein at least one of the additional therapeutic agents is an immune checkpoint modulator.

Embodiment 280. Use of an HDAC inhibitor in the manufacturing of a medicament for treating a cancer in a subject, wherein the treatment comprises administering to the subject an immune checkpoint modulator and an HDAC inhibitor, wherein the treatment modulates and/or improves the Teff cell to Treg cell ratio in the tumor or tumor microenvironment, wherein the cancer is identified as having modified STK11 activity or expression.

Embodiment 281. Use of an HDAC inhibitor in the manufacturing of a medicament for treating a cancer in a subject, wherein the treatment comprises administering to the subject an immune checkpoint modulator and an HDAC inhibitor, wherein the treatment reduces or depletes Treg cells in the tumor or tumor microenvironment, wherein the cancer is identified as having modified STK11 activity or expression.

Embodiment 282. Use of an HDAC inhibitor in the manufacturing of a medicament for treating a cancer in a subject, wherein the treatment comprises administering to the subject an immune checkpoint modulator and an HDAC inhibitor, wherein the treatment induces or increases the expression of cytokines that promote anti-tumor activity, wherein the cancer is identified as having modified STK11 activity or expression.

Embodiment 283. The use of embodiment 282, wherein the cytokines are selected from the group of CXCL9, CXCL10, and CXCL 11.

Embodiment 284. Use of an HDAC inhibitor in the manufacturing of a medicament for treating a cancer in a subject, wherein the treatment comprises administering to the subject an immune checkpoint modulator and an HDAC inhibitor, wherein the treatment reduces the expression of cytokines that promote Treg cell recruitment, wherein the cancer is identified as having modified STK11 activity or expression.

Embodiment 285. The use of embodiment 284, wherein the cytokines are CCL1 or CCL22.

Embodiment 286. Use of an HDAC inhibitor in the manufacturing of a medicament for treating a cancer in a subject, wherein the treatment comprises administering to the subject an HDAC inhibitor, wherein the administering of the HDAC inhibitor does not reduce erythroid or myeloid cell viability, wherein the cancer is identified as having modified STK11 activity or expression.

Embodiment 287. Use of an HDAC inhibitor in the manufacturing of a medicament for treating cancer in a subject, wherein the cancer presents an immune evasion phenotype characterized by STK11 mutant expression comprising: administering an HDAC1,2 selective inhibitor, wherein the HDAC1,2 selective inhibitor is capable of attenuating or reversing the immune evasion phenotype.

Embodiment 288. The use of embodiment 286 or 287, further comprising administering an immune checkpoint modulator.

Embodiment 289. Use of an HDAC inhibitor in the manufacturing of a medicament for treating a cancer in a subject, wherein the treatment comprises administering to the subject an HDAC inhibitor, wherein an immune checkpoint modulator has been, is, or will be administered to the subject, wherein the cancer is identified as having modified STK11 activity or expression.

Embodiment 290. Use of an HDAC inhibitor in the manufacturing of a medicament for treating a cancer in a subject, wherein the treatment comprises administering to the subject an immune checkpoint modulator, wherein an HDAC inhibitor has been, is, or will be administered to the subject, wherein the cancer is identified as having modified STK11 activity or expression.

Embodiment 291. The use of embodiment 277, wherein the HDAC inhibitor is administered in combination with two or more additional therapeutic agents, wherein at least two of the additional therapeutic agents are immune checkpoint modulators.

Embodiment 292. The use of any one of embodiments 279-290, wherein each immune checkpoint modulator is independently a checkpoint inhibitor, a T cell co-stimulatory receptor agonist or a dendritic cell co-stimulatory receptor agonist.

Embodiment 293. The use of any one of embodiments 279-292, wherein at least one immune checkpoint modulator is independently a T cell co-stimulatory receptor agonist or a dendritic cell co-stimulatory receptor agonist.

Embodiment 294. The use of any one of embodiments 279-292, wherein at least one immune checkpoint modulator is a checkpoint inhibitor.

Embodiment 295. The use of embodiment 294, wherein each checkpoint inhibitor is independently selected from an anti-CTLA-4 agent, an anti-PD-1 agent, an anti-PD-L1 agent, an anti-4-1 BB agent, an anti-OX-40 agent, an anti-GITR agent, an anti-CD27 agent, an anti-CD28 agent, an anti-CD40 agent, an anti-LAG3 agent, an anti-ICOS agent, an anti-TWEAKR agent, an anti-HVEM agent, an anti-TIM-1 agent, an anti-TIM-3 agent, an anti-VISTA agent, and an anti-TIGIT agent.

Embodiment 296. The use of embodiment 294, wherein each checkpoint inhibitor is independently selected from an anti-CTLA-4 agent, an anti-PD-1 agent and an anti-PD-L1 agent.

Embodiment 297. The use of embodiment 294, wherein each checkpoint inhibitor is independently selected from an anti-PD1 agent and an anti-PD-L1 agent.

Embodiment 298. The use of embodiment 294, wherein the checkpoint inhibitor is an anti-PD1 agent.

Embodiment 299. The use of embodiment 294, wherein the checkpoint inhibitor is an anti-PD-L1 agent.

Embodiment 300. The use of embodiment 277, wherein the HDAC inhibitor is administered in combination with an anti-CTLA-4 agent and an anti PD-1 or anti PD-L1 agent.

Embodiment 301. The use of any one of embodiments 294-299, wherein each immune checkpoint inhibitor is independently an antibody.

Embodiment 302. The use of embodiment 301, wherein each checkpoint inhibitor is independently selected from an anti-CTLA-4 antibody, an anti-PD-1 antibody, an anti-PD-L1 antibody, an anti-4-1 BB antibody, an anti-OX-40 antibody, an anti-GITR antibody, an anti-CD27 antibody, an anti-CD28 antibody, an anti-CD40 antibody, an anti-LAG3 antibody, an anti-ICOS antibody, an anti-TWEAKR antibody, an anti-HVEM antibody, an anti-TIM-1 antibody, an anti-TIM-3 antibody, an anti-VISTA antibody, and an anti-TIGIT antibody.

Embodiment 303. The use of embodiment 301, wherein each checkpoint inhibitor is independently selected from an anti-CTLA-4 antibody, an anti-PD-1 antibody and an anti-PD-L1 antibody.

Embodiment 304. The use of embodiment 301, wherein each immune checkpoint inhibitor is independently selected from nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab and geptanolimab.

Embodiment 305. The use of embodiment 301, wherein each checkpoint inhibitor is independently selected from an anti-PD1 antibody and an anti-PD-L1 antibody.

Embodiment 306. The use of embodiment 301, wherein the checkpoint inhibitor is an anti-PD1 antibody.

Embodiment 307. The use of embodiment 301, wherein the checkpoint inhibitor is an anti-PD1-L1 antibody.

Embodiment 308. The use of embodiment 302 or 303, wherein the anti-CTLA-4 antibody is ipilimumab.

Embodiment 309. The use of any one of embodiments 302, 303, 305 and 306, wherein the anti-PD-1 antibody is pembrolizumab or nivolumab.

Embodiment 310. The use of any one of embodiments 302, 303, 305 and 306, wherein the anti-PD-1 antibody is pembrolizumab.

Embodiment 311. The use of any one of embodiments 302, 303, 305 and 306, wherein the anti-PD-1 antibody is nivolumab.

Embodiment 312. The use of any one of embodiments 302, 303, 305 and 307, wherein the anti-PD-L1 antibody is atezolizumab (CAS number 1380723-44-3), avelumab (CAS number 1537032-82-8), or durvalumab (CAS number 1428935-60-7).

Embodiment 313. The use of any one of embodiments 279-291, wherein the immune checkpoint modulator is a T cell co-stimulatory receptor agonist or a dendritic cell co-stimulatory receptor agonist.

Embodiment 314. The use of any one of embodiments 278-313, wherein at least one additional therapeutic agent is targeted agent.

Embodiment 315. The use of embodiment 314, wherein each targeted agent is independently selected from an anti-angiogenesis agent (e.g., an anti-VEGF agent), a KRAS inhibitor, an ALK inhibitor, a ROS1 inhibitor, a BRAF inhibitor, a RET inhibitor, a MEK inhibitor, a MET inhibitor and a TRK inhibitor.

Embodiment 316. The use of embodiment 314, wherein each targeted agent is independently selected from bevacizumab, ramucirumab, sotorasib, crizotinib, ceritinib, alectinib, brigatinib, lorlatinib, entrectinib, dabrafenib, trametinib, capmatinib, tepotinib and larotrectinib.

Embodiment 317. The use of any one of embodiments 278-316, wherein at least one additional therapeutic agent is a chemotherapeutic agent.

Embodiment 318. The use of embodiment 317, wherein each chemotherapeutic agent is independently selected from cisplatin, carboplatin, paclitaxel, Albumin-bound paclitaxel (nab-paclitaxel), docetaxel, gemcitabine, vinorelbine, etoposide and pemetrexed.

Embodiment 319. The use of embodiment 317, wherein at least one chemotherapeutic agent is a platinum-containing therapeutic agent.

Embodiment 320. The use of embodiment 317, wherein one chemotherapeutic agent is a platinum-containing chemotherapeutic agent (e.g., cisplatin) and a second chemotherapeutic agent is pemetrexed.

Embodiment 321. The use of any one of embodiments 278-320, wherein at least one additional therapeutic agent is radiation.

Embodiment 322. The use of any one of embodiments 278-321, wherein the cancer is resistant to anti-PD1 therapy or anti-PD-L1 therapy.

Embodiment 323. The use of embodiment 322, wherein the cancer has intrinsic resistance to anti-PD1 therapy or anti-PD-L1 therapy.

Embodiment 324. The use of embodiment 322, wherein the cancer has acquired resistance to anti-PD1 therapy or anti-PD-L1 therapy.

Embodiment 325. The use of any one of embodiments 277-324, wherein the cancer is resistant to chemotherapy (e.g., platinum-containing chemotherapy).

Embodiment 326. The use of embodiment 325, wherein the cancer has intrinsic resistance to chemotherapy (e.g., platinum-containing chemotherapy).

Embodiment 327. The use of embodiment 325, wherein the cancer has acquired resistance to chemotherapy (e.g., platinum-containing chemotherapy).

Embodiment 328. The use of any one of embodiments 277-327, wherein the cancer does not respond to or benefit from treatment with an immune checkpoint modulator when administered alone or as part of a treatment regimen that does not include an HDAC inhibitor.

Embodiment 329. The use of any one of embodiments 277-328, wherein the cancer is selected from the group consisting of: lung cancer (e.g., lung adenocarcinoma, non-small cell lung cancer (NSCLC), squamous cell lung carcinoma), colorectal cancer (e.g., colon adenocarcinoma, rectal adenocarcinoma), breast cancer (e.g., invasive ductal carcinoma), pancreatic cancer (e.g., pancreatic adenocarcinoma), endometrial cancer (e.g., endometrioid carcinoma), neuroendocrine cancer (e.g., large cell neuroendocrine carcinoma), melanoma, non-melanoma skin cancer (e.g., skin squamous cell carcinoma), cholangiocarcinoma, gallbladder cancer, ovarian cancer (e.g., ovarian serous adenocarcinoma), bladder cancer (e.g., bladder urothelial carcinoma), prostate cancer (e.g., prostate adenocarcinoma), cervical cancer, endocervical cancer or cancer of unknown primary (e.g., adenocarcinoma of unknown primary).

Embodiment 330. The use of embodiment 329, wherein the cancer is lung cancer.

Embodiment 331. The use of embodiment 330, wherein the cancer is lung adenocarcinoma.

Embodiment 332. The use of embodiment 330, wherein the cancer is non-small cell lung cancer (NSCLC).

Embodiment 333. The use of embodiment 332, wherein the cancer is non-squamous non-small cell lung cancer (NSCLC).

Embodiment 334. The use of embodiment 329, wherein the cancer is colorectal cancer or colon adenocarcinoma.

Embodiment 335. The use of any one of embodiments 277-334, wherein the cancer is identified as having a STK11 mutation and one or more additional mutations.

Embodiment 336. The use of embodiment 335, wherein the additional mutations are selected from KRAS mutations and KEAP1 mutations.

Embodiment 337. The use of embodiment 335, wherein the additional mutations are KRAS mutations.

Embodiment 338. The use of embodiment 337, wherein the KRAS mutations are a mutations at position G12, optionally wherein the KRAS mutations are selected from G12D mutations, G12C mutations, G12V mutations or combinations thereof.

Embodiment 339. The use of embodiment 335, wherein the additional mutations are KEAP1 mutations.

Embodiment 340. The use of embodiment 335, wherein the additional mutations are KRAS mutations and KEAP 1 mutations.

Embodiment 341. The use of any one of embodiments 277-340, wherein the cancer does not have an EGFR mutation.

Embodiment 342. The use of any one of embodiments 277-341, wherein the cancer has increased or decreased STK11 expression.

Embodiment 343. The use of embodiment 342, wherein the increased or decreased STK expression is assessed by determining copy number of the gene encoding STK11 relative to a control sample, wherein an increase in the copy number indicates an increased level of expression and a decrease in the copy number indicates a decreased level of expression.

Embodiment 344. The use of embodiment 342, wherein the increased or decreased STK expression is assessed by determining the level of STK11 protein or mRNA relative to a control sample.

Embodiment 345. The use of embodiment 343 or 344, wherein the cancer has decreased STK11 expression.

Embodiment 346. The use of any one of embodiments 277-311, wherein the cancer has a STK11 mutation.

Embodiment 347. The use of embodiment 346, wherein the STK11 mutation is a mutation selected from:
(i) a mutation in the nucleotide sequence encoding STK11;
(ii) a mutation in a regulatory sequence controlling expression of the nucleotide sequence encoding STK11;
(iii) a mutation in a nucleotide encoding a protein which interacts with the transcription product of the STK11 gene.

Embodiment 348. The use of embodiment 346, wherein the STK11 mutation is a mutation in the translation product of the STK11 gene.

Embodiment 349. The use of embodiment 346, wherein the STK11 mutation is a mutation in the transcription product of the STK11 gene.

Embodiment 350. The use of any one of embodiments 346-349, wherein the STK11 mutation is an inactivating (loss of function) mutation.

Embodiment 351. The use of any one of embodiments 342-345, wherein the increased or decreased STK11 expression is determined in a sample derived from the subject.

Embodiment 352. The use of embodiment 351, wherein the increased or decreased STK11 expression is determined relative to a control.

Embodiment 353. The use of embodiment 352, wherein the control is healthy tissue, preferably of the same tissue type as the cancer tissue.

Embodiment 354. The use of any one of embodiments 346-350, wherein the STK11 mutation is identified in a sample derived from the subject.

Embodiment 355. The use of embodiment 354, wherein the STK11 mutation is identified in a tumor sample derived from the subject.

Embodiment 356. The use of embodiment 354, wherein the STK11 mutation is not identified in a healthy tissue sample derived from the subject.

Embodiment 357. The use of any one of embodiments 277-356, wherein the STK11 mutation is not a germline mutation.

Embodiment 358. The use of any one of embodiments 277-357, wherein the HDAC inhibitor is administered concurrently with the additional therapeutic agents.

Embodiment 359. The use of any one of embodiments 277-357, wherein the HDAC inhibitor is administered separately from the additional therapeutic agents.

Embodiment 360. The use of any one of embodiments 277-357, wherein the HDAC inhibitor is administered sequentially from the additional therapeutic agents.

Embodiment 361. The use of any one of embodiments 277-357, wherein the HDAC inhibitor is administered prior to the additional therapeutic agents.

Embodiment 362. The use of any one of embodiments 277-357, wherein the HDAC inhibitor is administered subsequent to the additional therapeutic agents.

Embodiment 363. The use of any one of embodiments 277-357, wherein the method comprises administering to the subject an immune checkpoint modulator followed by administering to the subject an HDAC inhibitor.

Embodiment 364. The use of any one of embodiments 277-357, wherein the method comprises administering to the subject an HDAC inhibitor followed by administering to the subject an immune checkpoint modulator.

Embodiment 365. The use of any one of embodiments 277-357, wherein the method comprises administering to the subject an immune checkpoint modulator, followed by administering to the subject a targeted agent, a chemotherapeutic agent, radiation or a combination thereof, followed by administering to the subject an HDAC inhibitor.

Embodiment 366. The use of any one of embodiments 277-357, wherein the method comprises administering to the subject an immune checkpoint modulator, followed by administering to the subject an HDAC inhibitor, followed by administering to the subject a targeted agent, a chemotherapeutic agent, radiation or a combination thereof.

Embodiment 367. The use of any one of embodiments 277-357, wherein the method comprises administering to the subject an HDAC inhibitor, followed by administering to the subject a targeted agent, a chemotherapeutic agent, radiation or a combination thereof, followed by administering to the subject an immune checkpoint modulator.

Embodiment 368. The use of any one of embodiments 277-357, wherein the method comprises administering to the subject an HDAC inhibitor, followed by administering to the subject an immune checkpoint modulator, followed by administering to the subject a targeted agent, a chemotherapeutic agent, radiation or a combination thereof.

Embodiment 369. The use of any one of embodiments 277-357, wherein the method comprises administering to the subject a targeted agent, a chemotherapeutic agent, radiation or a combination thereof, followed by administering to the subject an immune checkpoint modulator, followed by administering to the subject an HDAC inhibitor.

Embodiment 370. The use of any one of embodiments 277-357, wherein the method comprises administering to the subject a targeted agent, a chemotherapeutic agent, radiation or a combination thereof, followed by administering to the subject an HDAC inhibitor, followed by administering to the subject an immune checkpoint modulator.

Embodiment 371. The use of any one of embodiments 277-370, wherein the subject has a cancer identified as having modified STK11 activity or expression.

Embodiment 372. The use of any one of embodiments 277-371, wherein the histone deacetylase inhibitor is a selective inhibitor of histone deacetylase 1 (HDAC1-selective inhibitor).

Embodiment 373. The use of any one of embodiments 277-371, wherein the histone deacetylase inhibitor is a selective inhibitor of histone deacetylase 1 and histone deacetylase 2 (HDAC1,2-selective inhibitor).

Embodiment 374. The use of any one of embodiments 277-371, wherein the histone deacetylase inhibitor is a selective HDAC Class I inhibitor.

Embodiment 375. The use of any one of embodiments 277-371, wherein the histone deacetylase inhibitor is a CoREST-selective deacetylase inhibitor.

Embodiment 376. A method for ascertaining susceptibility of a subject having or having been diagnosed with cancer to treatment with an HDAC inhibitor, the method comprising: determining: i) the presence or absence of a STK11 mutation; and/or ii) the level of STK11 activity or expression in the subject or a sample derived from the subject; wherein the presence of a STK11 mutation and/or a modified level of STK11 activity or expression is indicative of susceptibility to treatment with an HDAC inhibitor.

Embodiment 377. A method for ascertaining susceptibility of a subject having or having been diagnosed with cancer to treatment with a combination of an HDAC inhibitor and an immune checkpoint modulator, the method comprising: determining: i) the presence or absence of a STK11 mutation; and/or ii) the level of STK11 activity or expression in the subject or a sample derived from the subject; wherein the presence of a STK11 mutation and/or a modified level of STK11 activity or expression is indicative of susceptibility to treatment with a combination of an HDAC inhibitor and an immune checkpoint modulator.

Embodiment 378. A method for ascertaining susceptibility of a subject having or having been diagnosed with cancer to treatment with a method of any one of embodiments 1-99, the method for ascertaining comprising: determining: i) the presence or absence of a STK11 mutation; and/or ii) the level of STK11 activity or expression in the subject or a sample derived from the subject: wherein the presence of a STK11 mutation and/or a modified level of STK11 activity or expression is indicative of susceptibility to treatment with a method of any one of embodiments 1-99.

Embodiment 379. The method of any one of embodiments 1-177, the compound for use of any one of embodiments 187-276 or the use of any one of embodiments 277-371 wherein the histone deacetylase inhibitor is a compound of Formula (I)

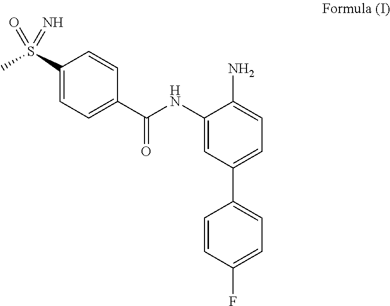 [SMILES: C[S@@](=N)(=O)c1ccc(C(=O)Nc2cc(-c3ccc(F)cc3)ccc2N)cc1]

or a pharmaceutically acceptable salt thereof.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Anti-Tumor Activity of Anti-PD1 and Compound I Combination in Murine Subcutaneous MC38 sgStk11 Tumor Model

Summary: An in vivo anti-tumor efficacy of double combination treatment of Anti-PD1 and HDAC1,2-selective inhibitor in a murine subcutaneous MC38_sgStk11 tumor model in 57BL/6 mice was performed.

Experimental Design: A table summarizing the experimental design is provided in Table 1-1.

TABLE 1-1
Summary of Experimental Design

| | | | | Dosing | |
|---|---|---|---|---|---|
| Group | N | Treatment | Dose^{a} (mg/kg) | Route | Schedule |
| 1 | 8 | Anti-IgG2a | 10 | IP | BIW x 7 weeks |
| 2 | 8 | Compound I | 3 | PO | QD x 49 days |
| | | Anti-IgG2a | 10 | IP | BIW x 7 weeks |
| 3 | 8 | Compound I | 10 | PO | QD x 49 days |
| | | Anti-IgG2a | 10 | IP | BIW x 7 weeks |
| 4 | 8 | Compound I | 30 | PO | QD x 49 days |
| | | Anti-IgG2a | 10 | IP | BIW x 7 weeks |
| 5 | 8 | Compound I | 75 | PO | QD x 49 days |
| | | Anti-IgG2a | 10 | IP | BIW x 7 weeks |
| 6 | 8 | Compound I | 150 (D 0-D 4) | PO | QD x 49 days |
| | | | 100 (D 5-D 48) | | |
| | | Anti-IgG2a | 10 | IP | BIW x 7 weeks |
| 7 | 8 | Anti-PD1 | 10 | IP | BIW x 7 weeks |
| 8 | 8 | Compound I | 3 | PO | QD x 49 days |
| | | Anti-PD1 | 10 | IP | BIW x 7 weeks |
| 9 | 8 | Compound I | 10 | PO | QD x 49 days |
| | | Anti-PD1 | 10 | IP | BIW x 7 weeks |
| 10 | 8 | Compound I | 30 | PO | QD x 49 days |
| | | Anti-PD1 | 10 | IP | BIW x 7 weeks |
| 11 | 8 | Compound I | 75 | PO | QD x 49 days |
| | | Anti-PD1 | 10 | IP | BIW x 7 weeks |
| 12 | 8 | Compound I | 150 (D 0-D 4) | PO | QD x 49 days |
| | | | 100 (D 5-D 48) | | |
| | | Anti-PD1 | 10 | IP | BIW x 7 weeks |

^{a}Dosing volume for each dose was 10 μL/g.

### Materials:

### Animals

96 mice plus 39 spare mice of the species: Mus musculus: strain: C57BL/6; age 8-10 weeks; sex: female; and body weight: 16.9-20.0 g

Animal supplier: Shanghai SLAC Laboratory Animal Co., Ltd

The mice were kept in individual ventilation cages at constant temperature (about 20-26° C.) and humidity (about 40-70%) with 4 animals in each cage. The size of each cage was about 300 mm×200 mm×180 mm. The bedding material in each cage was corn cob, which was changed twice per week. The identification labels for each cage contained the following information: number of animals, sex, strain, date received, treatment, study number, group number and the starting date of the treatment.

Animals had free access to irradiation sterilized dry granule food during the entire study period. Animals had free access to sterile drinking water. Animals were identified by ear tags.

### Compounds

Anti-PD1 (solution) supplied by BioXcell and stored at about 4° C. Anti-IgG2a (solution) supplied by BioXcell and stored at about 4° C. Compound I (solid) supplied by Tango Therapeutics and stored at about room temperature. Compound I used in Example 1 exhibits a >10-fold selectivity for HDAC1 compared to HDAC3 in intact cells. Compound I used in Example 1 exhibits a significant selectivity for CoREST deacetylase compared to NCOR, NuRD, and Sin3 as described herein.

### Methods

### Cell Culture

The MC38_sgStk11 tumor cells were maintained in vitro as a monolayer culture in DMEM+2 mM glutamine supplemented with about 10% heat inactivated fetal bovine serum, about 100 U/ml penicillin and 100 μg/ml streptomycin at about 37° C. in an atmosphere of about 5% CO₂ in air. The tumor cells were routinely sub-cultured twice weekly by trypsin-EDTA treatment. The cells growing in an exponential growth phase were harvested and counted for tumor inoculation.

### Tumor Inoculation and Animal Grouping

Each mouse was inoculated subcutaneously at the right upper flank with MC38_sgStk11 tumor cells (0.5×106) in 0.1 ml of PBS for tumor development. Treatments were started on day 4 after tumor inoculation when the average tumor size reached 52 mm³. The animals were assigned into groups according to a sorting standard operating procedures based upon their tumor volumes. Each group consisted of 8 tumor bearing mice. The testing article was administrated to the mice according to the predetermined regimen as shown in the experimental design table (Table 1-1).

### Observations

All the procedures related to animal handling, care and the treatment in the study were performed according to the guidelines approved by the Institutional Animal Care and Use Committee (IACUC) of vendor following the guidance of the Association for Assessment and Accreditation of Laboratory Animal Care (AAALAC). At the time of routine monitoring, the animals were daily checked for any effects of tumor growth and treatments on normal behavior such as mobility, food and water consumption, body weight gain/loss (body weights were measured three times per week), eye/hair matting and any other abnormal effect as stated in the protocol. Death and observed clinical signs were recorded on the basis of the numbers of animals within each subset.

### Tumor Measurements and Endpoints

A primary endpoint was to see if the tumor growth could be delayed or mice could be cured. Tumor size was measured three times per week in two dimensions using a caliper, and the volume was expressed in mm3 using the formula: V=0.5 a×b2 where a and b are the long and short diameters of the tumor, respectively. The T/C value (in percent) is an indication of antitumor effectiveness; T and C are the mean volume of the treated and control groups, respectively, on a given day. TGI is calculated for each group using the formula: TGI (%)=[1−(Ti−T0)/(Vi−V0)]×100; Ti is the average tumor volume of a treatment group on a given day, T0 is the average tumor volume of the treatment group on the day of treatment start, Vi is the average tumor volume of the vehicle control group on the same day with Ti, and V0 is the average tumor volume of the vehicle group on the day of treatment start.

### Statistical Analysis

Statistical analysis of difference in the tumor volume among the groups were conducted on the data obtained at the best therapeutic time point at the day 11 after the start of treatment. A one-way ANOVA was performed for comparisons between groups. The survival analysis between groups was carried out with Kaplan-Meier test. p<0.05 was considered to be statistically significant.

### Results

Mean tumor volume over time in C57BL/6 mice bearing MC38_sgStk11 tumors dosed with combination treatment is shown in Table 1-2 (Anti-IgG2a) and Table 1-3 (Anti-PD1).

TABLE 1-2
Tumor Volume (mm³) Per Anti-IgG2a Group Over Time
(includes measurements for surviving animals only)

| | Tumor Volume (mm³)^{a} by Group | | | | | |
|---|---|---|---|---|---|---|
| | | | | | | 6 |
| | | | | | | Anti-IgG2a |
| | | | | | | Compound |
| | | 2 | | | | I (150 |
| | | Anti-IgG2a | 3 | 4 | 5 | mg/kg D0- |
| | 1 | Compound | Anti-IgG2a | Anti-IgG2a | Anti-IgG2a | D4 |
| | Anti- | I | Compound I | Compound I | Compound I | 100 mg/kg |
| Days^{b} | IgG2a | (3 mg/kg) | (10 mg/kg) | (30 mg/kg) | (75 mg/kg) | D5-D48) |
| 0 | 52 ± 2 | 52 ± 2 | 52 ± 2 | 52 ± 2 | 52 ± 2 | 52 ± 2 |
| 2 | 218 ± 12 | 208 ± 6 | 213 ± 10 | 212 ± 14 | 218 ± 12 | 205 ± 14 |
| 4 | 496 ± 39 | 489 ± 23 | 473 ± 38 | 484 ± 37 | 485 ± 37 | 450 ± 40 |
| 7 | 906 ± 95 | 925 ± 98 | 735 ± 82 | 734 ± 64 | 576 ± 45 | 508 ± 40 |
| 9 | 1506 ± 180 | 1439 ± 130 | 1059 ± 167 | 883 ± 84 | 622 ± 44 | 636 ± 19 |
| 11 | 2132 ± 344 | 2105 ± 184 | 1403 ± 270 | 1055 ± 101 | 632 ± 55 | 878 ± 66 |
| 14 | 2602 ± 251 | 2876 ± 196 | 1923 ± 343 | 1481 ± 146 | 580 ± 85 | 1090 ± 167 |
| 16 | 3052 ± 128 | 3215 ± 79 | 2101 ± 176 | 1977 ± 272 | 557 ± 101 | 1236 ± 211 |
| 18 | 3032 | EU | 2570 ± 205 | 2344 ± 368 | 536 ± 109 | 1414 ± 243 |
| 21 | EU | EU | 3275 ± 64 | 2602 ± 361 | 514 ± 117 | 1728 ± 366 |
| 23 | EU | EU | EU | 2794 ± 665 | 502 ± 138 | 2198 ± 472 |
| 25 | EU | EU | EU | 1551 | 477 ± 151 | 2054 ± 638 |
| 28 | EU | EU | EU | 2589 | 551 ± 171 | 1705 ± 330 |
| 30 | EU | EU | EU | 3550 | 815 ± 291 | 1840 ± 326 |
| 32 | EU | EU | EU | EU | 1001 ± 375 | 1949 ± 395 |
| 34 | EU | EU | EU | EU | 827 ± 268 | 2089 ± 389 |
| 38 | EU | EU | EU | EU | 14 | EU |
| 42 | EU | EU | EU | EU | 13 | EU |
| 45 | EU | EU | EU | EU | 6 | EU |
| 49 | EU | EU | EU | EU | 0 | EU |
| 52 | EU | EU | EU | EU | 0 | EU |
| 56 | EU | EU | EU | EU | 0 | EU |
| 59 | EU | EU | EU | EU | 0 | EU |
| 63 | EU | EU | EU | EU | 0 | EU |
| 66 | EU | EU | EU | EU | 0 | EU |

^{a}Mean ± SEM
^{b}Days after the start of treatment
EU = Mice were euthanized in advance for heavy tumor burden (>3,000mm³)

TABLE 1-3
Tumor Volume (mm³) Per Anti-PD1 Group Over Time (includes
measurements for surviving animals only)

| | Tumor Volume (mm3)^{a} by Group | | | | | |
|---|---|---|---|---|---|---|
| | | | | | | 12 |
| | | | | | | Anti-PD1 |
| | | 8 | 9 | 10 | | Compound I |
| | | Anti-PD1 | Anti-PD1 | Anti-PD1 | 11 | (150 mg/kg |
| | | Compound | Compound | Compound | Anti-PD1 | D0-D4 |
| | 7 | I | I (10 | I (30 | Compound I | 100 mg/kg D5- |
| Days^{b} | Anti-PD1 | (3 mg/kg) | mg/kg) | mg/kg) | (75 mg/kg) | D48) |
| 0 | 52 ± 2 | 52 ± 2 | 52 ± 2 | 52 ± 2 | 52 ± 2 | 52 ± 2 |
| 2 | 226 ± 12 | 218 ± 9 | 217 ± 11 | 218 ± 12 | 223 ± 11 | 213 ± 13 |
| 4 | 495 ± 43 | 453 ± 33 | 414 ± 23 | 407 ± 35 | 406 ± 40 | 402 ± 19 |
| 7 | 848 ± 109 | 631 ± 100 | 669 ± 91 | 498 ± 74 | 547 ± 69 | 443 ± 48 |
| 9 | 1042 ± 144 | 703 ± 141 | 859 ± 175 | 491 ± 92 | 580 ± 96 | 512 ± 83 |
| 11 | 1127 ± 189 | 708 ± 155 | 884 ± 247 | 399 ± 109 | 542 ± 125 | 668 ± 110 |
| 14 | 1286 ± 198 | 796 ± 225 | 678 ± 269 | 285 ± 99 | 503 ± 156 | 823 ± 159 |
| 16 | 1471 ± 221 | 983 ± 300 | 612 ± 281 | 245 ± 105 | 557 ± 242 | 973 ± 241 |
| 18 | 1878 ± 308 | 1252 ± 416 | 625 ± 275 | 226 ± 112 | 598 ± 311 | 1111 ± 300 |
| 21 | 2039 ± 415 | 889 ± 331 | 800 ± 309 | 205 ± 115 | 583 ± 386 | 1249 ± 345 |
| 23 | 1696 ± 305 | 1226 ± 527 | 1017 ± 390 | 196 ± 120 | 169 ± 94 | 1406 ± 431 |
| 25 | 2127 ± 494 | 972 ± 461 | 1227 ± 480 | 198 ± 125 | 118 ± 64 | 1162 ± 403 |
| 28 | 2399 ± 707 | 1383 ± 710 | 1213 ± 565 | 234 ± 144 | 93 ± 56 | 1213 ± 458 |
| 30 | 1511 ± 437 | 1042 ± 730 | 997 ± 623 | 269 ± 166 | 71 ± 42 | 1428 ± 576 |
| 32 | 2115 ± 688 | 377 ± 236 | 618 ± 601 | 353 ± 222 | 66 ± 44 | 1148 ± 613 |
| 34 | 2557 ± 702 | 547 ± 329 | 11 ± 3 | 385 ± 246 | 77 ± 60 | 660 ± 525 |
| 38 | EU | 6 | 9 ± 3 | 115 | 15 + 3 | 14 ± 8 |
| 42 | EU | 6 | 9 ± 3 | 10 ± 4 | 12 ± 4 | 12 ± 6 |
| 45 | EU | 6 | 8 ± 2 | 8 ± 4 | 12 ± 4 | 9 ± 3 |
| 49 | EU | 6 | 6 ± 0 | 5 ± 4 | 18 ± 10 | 9 ± 3 |
| 52 | EU | 6 | 2 ± 2 | 5 ± 5 | 30 ± 24 | 6 ± 0 |
| 56 | EU | 6 | 2 ± 2 | 5 ± 5 | 2 ± 2 | 3 ± 3 |
| 59 | EU | 0 | 2 ± 2 | 9 ± 9 | 1 ± 1 | 0 ± 0 |
| 63 | EU | 0 | 0 ± 0 | 0 ± 0 | 0 ± 0 | 0 ± 0 |
| 66 | EU | 0 | 0 ± 0 | 0 ± 0 | 0 ± 0 | 0 ± 0 |

^{a}Mean ± SEM
^{b}Days after the start of treatment
EU = Mice were euthanized in advance for heavy tumor burden (>3,000mm³)

TABLE 1-4
Tumor Growth Inhibition Calculation in the
MC38_sgStk11 Syngeneic Model Calculated
Based on Tumor Volume Measurements at PG-D11

| | Tumor Size | | | p value^{d} |
|---|---|---|---|---|
| | (mm³)^{a} at | T/C ^{b} | TGI %^{c} | One-way |
| Treatment | Day 11 | (%) | (%) | ANOVA |
| 1 Anti-IgG2a | 2132 ± 344 | — | — | — |
| 2 Anti-IgG2a | 2105 ± 184 | 98.7 | 1.3 | 1.000 |
| Compound I | | | | |
| (3 mg/kg) | | | | |
| 3 Anti-IgG2a | 1403 ± 270 | 65.8 | 35.0 | 0.059 |
| Compound I | | | | |
| (10 mg/kg) | | | | |
| 4 Anti-IgG2a | 1055 ± 101 | 49.5 | 51.8 | 0.001 |
| Compound I | | | | |
| (30 mg/kg) | | | | |
| 5 Anti-IgG2a | 632 ± 55 | 29.7 | 72.1 | <0.001 |
| Compound I | | | | |
| (75 mg/kg) | | | | |
| 6 Anti-IgG2a | 878 ± 66 | 41.2 | 60.2 | 0.002 |
| Compound I | | | | |
| (150 mg/kg | | | | |
| D 0-D 4 | | | | |
| 100 mg/kg | | | | |
| D 5-D 48) | | | | |
| 7 Anti-PD1 | 1127 ± 189 | 52.9 | 48.3 | 0.003 |
| 8 Anti-PD1 | 708 ± 155 | 33.2 | 68.5 | <0.001 |
| Compound I | | | | |
| (3 mg/kg) | | | | |
| 9 Anti-PD1 | 884 ± 247 | 41.5 | 60.0 | <0.001 |
| Compound I | | | | |
| (10 mg/kg) | | | | |
| 10 Anti-PD1 | 399 ± 109 | 18.7 | 83.3 | <0.001 |
| Compound I | | | | |
| (30 mg/kg) | | | | |
| 11 Anti-PD1 | 542 ± 125 | 25.4 | 76.4 | <0.001 |
| Compound I | | | | |
| (75 mg/kg) | | | | |
| 12 Anti-PD1 | 668 ± 110 | 31.3 | 70.4 | <0.001 |
| Compound I | | | | |
| (150 mg/kg | | | | |
| D 0-D 4 | | | | |
| 100 mg/kg | | | | |
| D 5-D 48) | | | | |

^{a}Mean ± SEM.
^{b} Tumor Growth Inhibition is calculated by dividing the group average tumor volume for the treated group by the group average tumor volume for the control group (T/C).
^{c}TGI (%) = [1 − (T11 − T0)/(V11 − V0)] × 100; T11 is the average tumor volume of a treatment group on day 11, T0 is the average tumor volume of the treatment group on day 0 after treatment, V11 is the average tumor volume of the vehicle control group on the same day with T11, and V0 is the average tumor volume of the vehicle group on day 0 after treatment.
^{d}All data were analyzed using SPSS 17.0. Comparisons between groups were carried out with One-Way ANOVA, followed by Games-Howell (equal variances not assumed) or Dunnett (equal variances assumed) test.
* p ≤ 0.05;
** p ≤ 0.01;
*** p ≤ 0.001; p ≤ 0.0001.

TABLE 1-5
Tumor Regression Responses Analysis in the MC38_sgStk11
Syngeneic Model Calculated Based on Tumor Volume Measurements

| Treatment | PR^{a} (%) | CR^{b} (%) | ORR^{c} (%) |
|---|---|---|---|
| 1 Anti-IgG2a | 0.0 | 0.0 | 0.0 |
| 2 Anti-IgG2a | 0.0 | 0.0 | 0.0 |
| Compound I | | | |
| (3 mg/kg) | | | |
| 3 Anti-IgG2a | 0.0 | 0.0 | 0.0 |
| Compound I | | | |
| (10 mg/kg) | | | |
| 4 Anti-IgG2a | 0.0 | 0.0 | 0.0 |
| Compound I | | | |
| (30 mg/kg) | | | |
| 5 Anti-IgG2a | 0.0 | 12.5 | 12.5 |
| Compound I | | | |
| (75 mg/kg) | | | |
| 6 Anti-IgG2a | 0.0 | 0.0 | 0.0 |
| Compound I | | | |
| (150 mg/kg | | | |
| D 0-D 4 | | | |
| 100 mg/kg | | | |
| D 5-D 48) | | | |
| 7 Anti-PD1 | 0.0 | 0.0 | 0.0 |
| 8 Anti-PD1 | 0.0 | 12.5 | 12.5 |
| Compound I | | | |
| (3 mg/kg) | | | |
| 9 Anti-PD1 | 0.0 | 50.0 | 50.0 |
| Compound I | | | |
| (10 mg/kg) | | | |
| 10 Anti-PD1 | 12.5 | 62.5 | 75.0 |
| Compound I | | | |
| (30 mg/kg) | | | |
| 11 Anti-PD1 | 12.5 | 62.5 | 75.0 |
| Compound I | | | |
| (75 mg/kg) | | | |
| 12 Anti-PD1 | 0.0 | 25.0 | 25.0 |
| Compound I | | | |
| (150 mg/kg | | | |
| D 0-D 4 | | | |
| 100 mg/kg | | | |
| D 5-D 48) | | | |

^{a}PR (partial regression) is the tumor volume was 50% or less of its Day1 volume for three consecutive measurements during the course of the study, and equal to or greater than 13.5 mm3 for one or more of these three measurements.
^{b}CR (complete regression) is the tumor volume was less than 13.5 mm3 for three consecutive measurements during the course of the study.
^{c}ORR = CR (complete response) + PR (partial response).

Combination treatment of Compound I with Anti-PD1 produced significant antitumor activity against the MC38_sgStk11 tumor model. The mean tumor size of the Vehicle treated animals reached 2,132 mm³ at day 11 after the start of treatment. Combination treatment of Anti-PD1 and Compound I at different dose levels (3 mg/kg, 10 mg/kg, 30 mg/kg and 75 mg/kg) produced significant anti-tumor activities, their mean tumor sizes were about 708 mm³, 884 mm³, 399 mm³ and 542 mm³ at the same time (T/C value=33.2%, 41.5%, 18.7% and 25.4%; TGI=68.5%, 60.0%, 83.3% and 76.4%; p<0.001, <0.001, <0.001 and <0.001 respectively, compared with the Vehicle group).

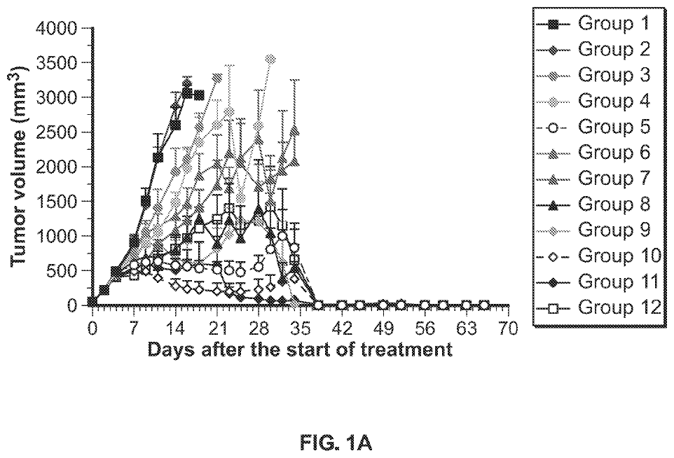

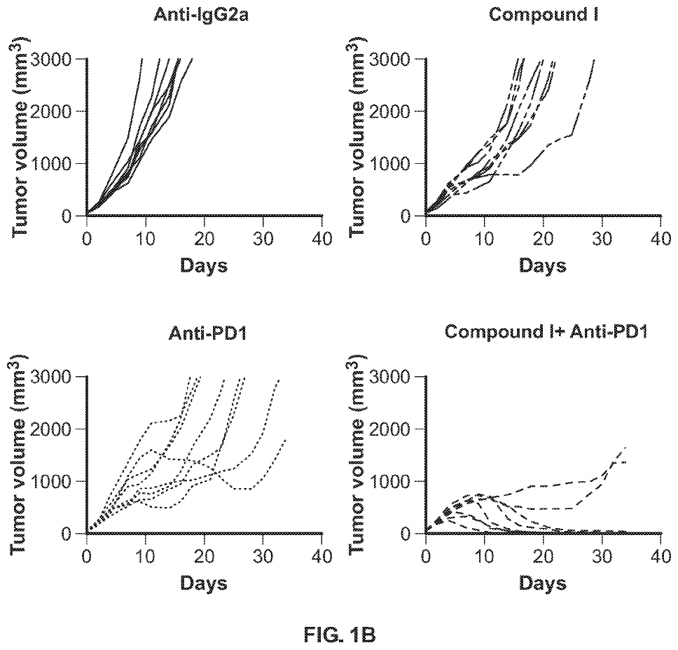

Tumor volume and survival was monitored over the course of treatment. The tumor volume was plotted by treatment group (FIG. 1A) and by individual animal (FIG. 1B).

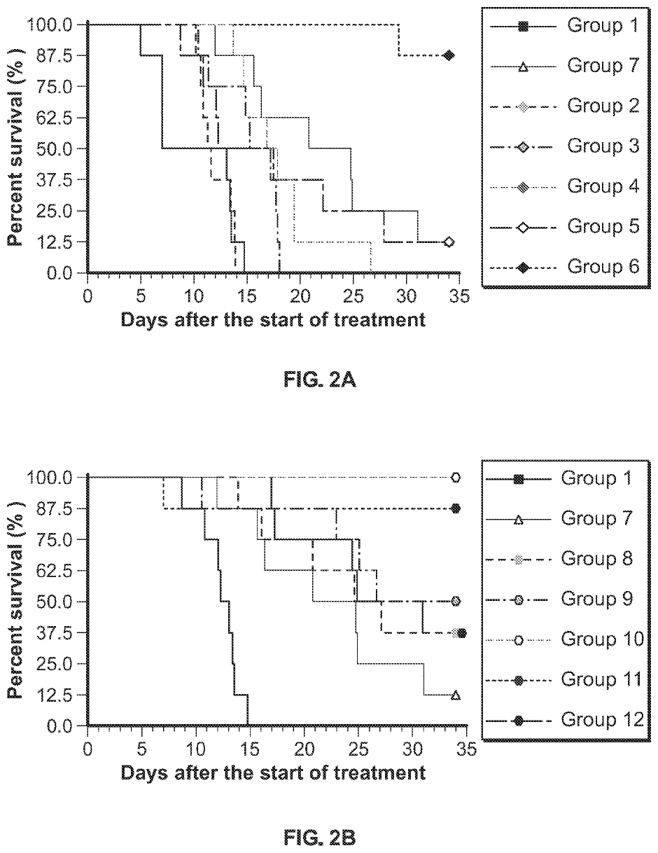

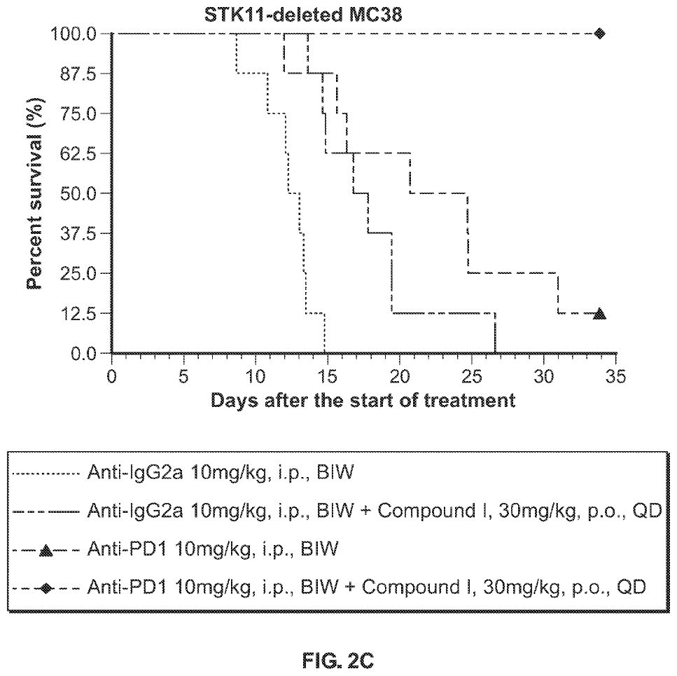

Survival was plotted by tumor group (FIG. 2A, showing groups 1, 7, 2, 3, 4, 5 and 6; FIG. 2B, showing groups 1, 7, 8, 9, 10, 11 and 12; FIG. 2C showing groups 1, 4, 7 and 10).

### Example 2: Re-Challenge Study of Example 1

Summary: A re-challenge study of Example 1 was performed to evaluate whether the survived animals from Example 1 had acquired T memory against the same tumor.

Experimental Design & Materials: The survived animals of the efficacy part were re-challenged with MC38_sgStk11 cells respectively in the opposite side of primary tumors.

A table summarizing the survived animals from Example 1 is provided in Table 2-1

TABLE 2-1
Summary of Animals in Re-Challenge Study

| | | Number of |
|---|---|---|
| Re-Challenge Group | | Animals |
| Group | Description | per Group |
| 1 | Untreated Control | 8 |
| 2 | Survived Animals from Group 5 of Example 1 | 1 |
| | Anti-IgG2a Compound I (75 mg/kg) | |
| 3 | Survived Animals from Group 8 of Example 1 | |
| | Anti-PD1 Compound I (3 mg/kg) | 1 |
| 4 | Survived Animals from Group 9 of Example 1 | 4 |
| | Anti-PD1 Compound I (10 mg/kg) | |
| 5 | Survived Animals from Group 10 of Example 1 | 5 |
| | Anti-PD1 Compound I (30 mg/kg) | |
| 6 | Survived Animals from Group 11 of Example 1 | 5 |
| | Anti-PD1 Compound I (75 mg/kg) | |
| 7 | Survived Animals from Group 12 of Example 1 | 2 |
| | Anti-PD1 Compound I (150 mg/kg D 0-D 4 | |
| | \| 100 mg/kg D 5-D 48) | |

### Methods

### Cell Culture

The MC38_sgStk11 tumor cells were maintained in vitro as a monolayer culture in DMEM+2 mM glutamine supplemented with about 10% heat inactivated fetal bovine serum, 100 U/ml penicillin and 100 μg/ml streptomycin at about 37° C. in an atmosphere of about 5% CO₂ in air. The tumor cells were routinely sub-cultured twice weekly by trypsin-EDTA treatment. The cells growing in an exponential growth phase were harvested and counted for tumor inoculation.

### Tumor Inoculation and Animal Grouping

Mice were inoculated subcutaneously at the left lower flank with MC38_sgStk11 tumor cells (0.5×106) in 0.1 ml of PBS for tumor development. The animal numbers and the cell numbers for each group are shown in the experimental design (Table 2-1). The survived animals from the Example 1 were under post-treatment monitoring for 21 days before re-challenge.

### Observations

All the procedures related to animal handling, care and the treatment in the study were performed according to the guidelines approved by the Institutional Animal Care and Use Committee (IACUC) of vendor following the guidance of the Association for Assessment and Accreditation of Laboratory Animal Care (AAALAC). At the time of routine monitoring, the animals were daily checked for any effects of tumor growth and treatments on normal behavior such as mobility, food and water consumption, body weight gain/loss (body weights were measured twice per week), eye/hair matting and any other abnormal effect as stated in the protocol. Death and observed clinical signs were recorded on the basis of the numbers of animals within each subset.

### Tumor Measurements and Endpoints

Tumor size was measured three times per week in three dimensions using a caliper, and the volume was expressed in mm3 using the formula: V=0.5 a×b2 where a and b are the long and short diameters of the tumor, respectively.

### Results

Mean tumor volume over time in C57BL/6 mice re-challenged with MC38_sgStk11 is shown in Table 2-2.

TABLE 2-2
Tumor Volume (mm³) Per MC38_sgStk11 Re-Challenge Group Over Time

| | Tumor Volume (mm3)^{a} by Group | | | | | | |
|---|---|---|---|---|---|---|---|
| | | | | | | | 7 |
| | | | | | | | (Ex. 1 Gr. 12) |
| | | | | | | | Anti-IgG2a |
| | | | | 4 | | | Compound |
| | | 2 | 3 | (Ex. 1 Gr. 9) | 5 | 6 | I (150 |
| | | (Ex. 1 Gr. 5) | (Ex. 1 Gr. 8) | Anti-PD1 | (Ex. 1 Gr. 10) | (Ex. 1 Gr. 11) | mg/kg D0- |
| | 1 | Anti-IgG2a | Anti-PD1 | Compound | Anti-PD1 | Anti-PD1 | D4 |
| | Untreated | Compound | Compound I | I (10 | Compound I | Compound I | 100 mg/kg |
| Days^{b} | Control | I (75 mg/kg) | (3 mg/kg) | mg/kg) | (30 mg/kg) | (75 mg/kg) | D5-D48) |
| 0 | 0 ± 0 | 0 | 0 ± 0 | 0 ± 0 | 0 ± 0 | 0 ± 0 | 0 ± 0 |
| 3 | 32 ± 2 | 17 | 18 | 22 ± 6 | 22 ± 5 | 26 ± 5 | 27 ± 2 |
| 5 | 45 ± 3 | 25 | 21 | 23 ± 6 | 21 ± 5 | 27 ± 6 | 27 ± 1 |
| 8 | 283 ± 40 | 6 | 6 | 8 ± 2 | 8 ± 2 | 12 ± 5 | 6 ± 0 |
| 10 | 565 ± 85 | 0 | 0 | 3 ± 2 | 2 ± 1 | 7 ± 6 | 0 ± 0 |
| 12 | 794 ± 114 | 0 | 0 | 0 ± 0 | 0 ± 0 | 5 ± 5 | 0 ± 0 |
| 15 | 1304 ± 228 | 0 | 0 | 0 ± 0 | 0 ± 0 | 3 ± 3 | 0 ± 0 |
| 17 | 1809 ± 340 | 0 | 0 | 0 ± 0 | 0 ± 0 | 1 ± 1 | 0 ± 0 |
| 19 | 2005 ± 437 | 0 | 0 | 0 ± 0 | 0 ± 0 | 0 ± 0 | 0 ± 0 |

^{a}Mean ± SEM
^{b}Days after cell inoculation

There was no tumor growth on the survived animals re-challenged with MC38_sgStk11 cells. The results may suggest that the survived animals had acquired T memory against MC38_sgStk11 tumor.

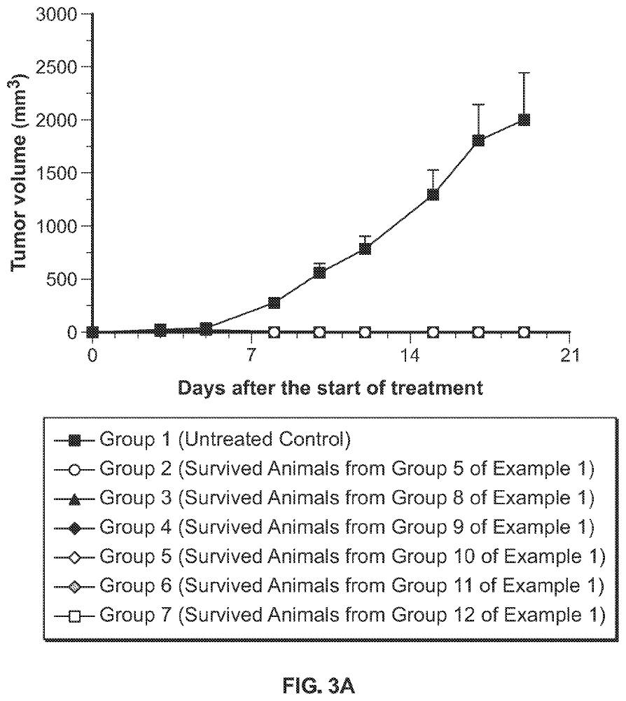

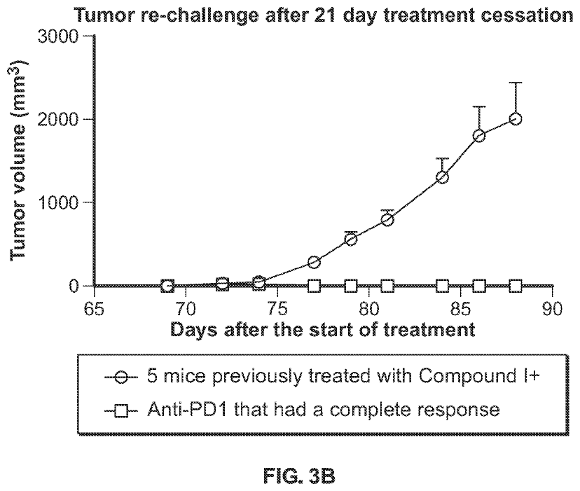

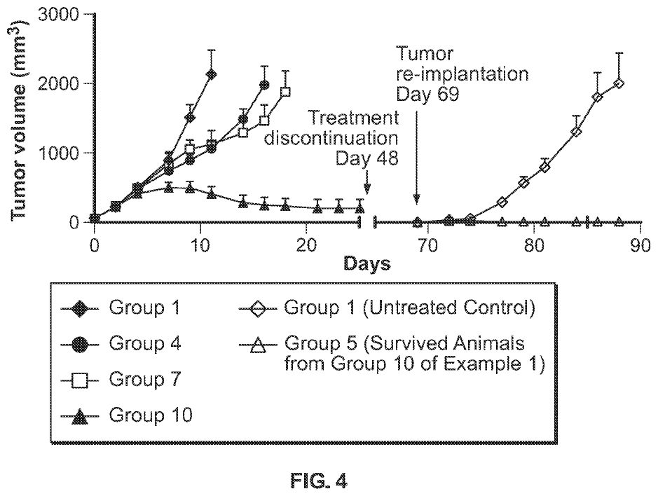

All animals remained off-treatment, and tumor size was plotted over time after re-challenge: FIG. 3A, shows tumor size by groups; all previously treated groups overlap with no tumor growth; FIG. 3B shows tumor size for a pooled group of previously treated mice vs. untreated control group). FIG. 4 shows a complete timeline and tumor sizes at the time points indicated for experiments 1 and 2.

### Example 3: Identification of Anti-PD1 Antibody Sensitizer Genes in STK-11 Deleted Tumors

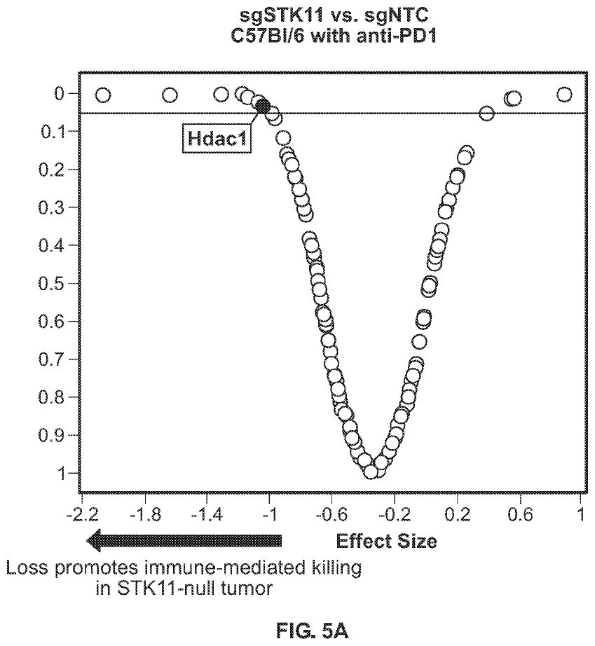

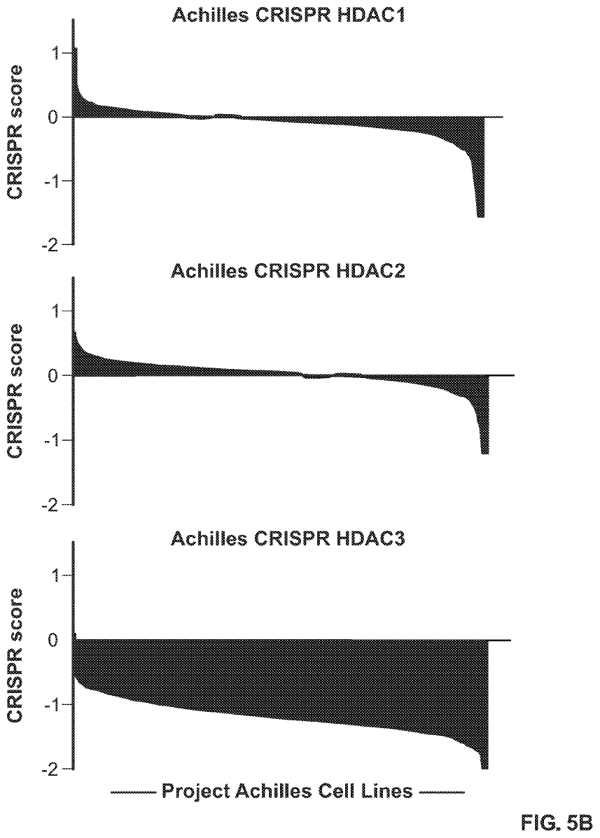

In this example, genes whose inhibition reverses anti-PD1 resistance driven by loss of STK11 were determined. Briefly, the genes coding for HDAC1, HDAC2, or HDAC3 were deleted or disabled with CRISPR in an in vivo knockout screen in STK-11 deleted MC38 tumor cells grown in C57Bl/6 mice that were treated with anti-PD1 antibody. The results show that HDAC1 is a sensitizer to anti-PD1 in STK11-deleted cancer (FIG. 5A). Additionally, toxicity of HDAC1, HDAC2, or HDAC3 by CRISPR mediated deletion/disruption in several cell lines was tested. The results show depletion of cells with HDAC1, HDAC2, or HDAC3 knockout, with HDAC3 knockout showing the highest toxicity in the panel of cell lines (FIG. 5B).

### Example 4: Identification of CoREST-Selective Deacetylase Inhibitors

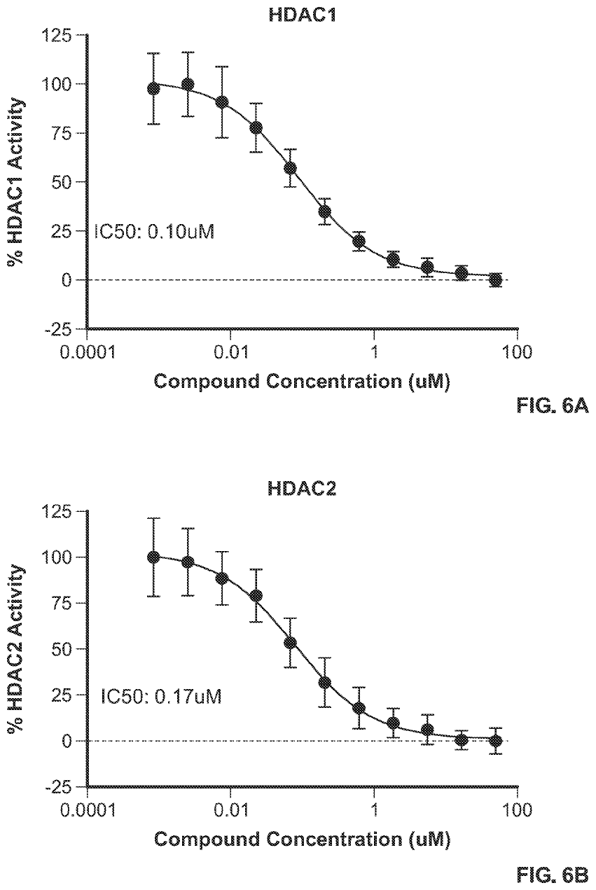

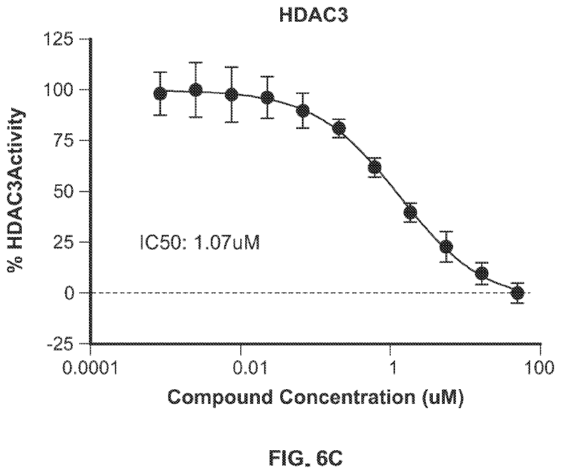

In this example, a CoREST complex selective Compound I was identified using an NanoBRET (Bioluminescence resonance energy transfer) target engagement assay. Briefly, cells were treated with Compound 1 for 4 hours prior to measuring the BRET signal. The dose-dependent binding of Compound I to purified HDAC1, HDAC2, and HDAC3 was then measured at various concentrations of Compound I and the respective IC50 values determined. The results show that the IC50s of Compound I for HDAC1 is 0.01 uM, HDAC2 0.17 uM, and HDAC3 1.07 uM (FIGS. 6A-6C, and Table 3-1).

TABLE 3-1
Compound I and other less-selective HDAC inhibitors (TCA,
Entinostat, and Chidamide) were evaluated in a cellular
NanoBRET assay for inhibition of HDAC 1, 2, 3, 6 and 10

| Compound | HDAC1 | HDAC2 | HDAC3 | HDAC6 | HDAC10 |
|---|---|---|---|---|---|
| Compound I | 0.10 | 0.17 | 1.07 | >30 | >30 |
| TCA | 0.01 | 0.02 | 0.08 | 5.09 | 0.20 |
| Entinostat | 1.45 | 0.38 | 1.12 | >30 | >30 |
| Chidamide | 0.84 | 0.25 | 0.41 | >30 | >30 |

HDAC inhibition of the HDAC complexes CoREST, NCOR, NuRD and Sin3 by Compound I was determined by a fluorescence-based deacetylase assay. Briefly, the HDAC complexes COREST, NCOR, NuRD and Sin3 were co-immunoprecipitated with complex selective antibodies from A549 cells (a model for lung adenocarcinoma), the complexes were incubated with Compound I or the less selective HDAC inhibitors Vorinostat, Tucidinostat, and Domatinostat. Suberoylanilide hydroxamic acid, a pan HDAC inhibitor that targets all 4 complexes, was used as a positive control for complex activity to demonstrate that the isolated complexes retained functional deacetylase activity, and to establish background assay fluorescence. The HDAC inhibition was profiled in a fluorescence-based deacetylase assay. Exemplary IC₅₀s are shown in Table 3-2. The results in Table 3-2 show that Compound I is selective for the CoREST complex.

TABLE 3-2
IC50's for Compound I and other HDAC inhibitors (Vorinostat,
Tucidinostat, Domatinostat) in an in vitro deacetylase assay of
cell-derived, intact HDAC complexes, CoREST, NCoR, NuRD, and Sin3

| Compound | CoREST | NCoR | NuRD | Sin3 |
|---|---|---|---|---|
| Vorinostat | 0.061 | 0.525 | 0.133 | 0.160 |
| Tucidinostat | 0.120 | 0.2 | 1.4 | >100 |
| Domatinostat | 0.087 | 0.193 | 30 | >100 |
| Compound 1 | 0.170 | >100 | >100 | >100 |

### Example 5: Anti-Tumor Activity of Anti-PD1 in Combination with Compound I in Murine Subcutaneous CT26 STK11-deleted Tumor Models

In this example, the anti-tumor activity of a combination treatment of anti-PD1 antibody with Compound I was determined in vivo in STK11-deleted syngeneic mouse models.

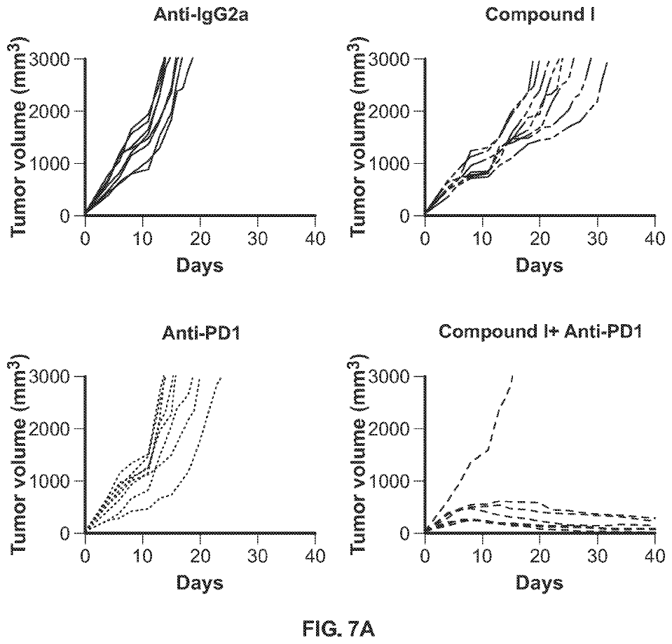

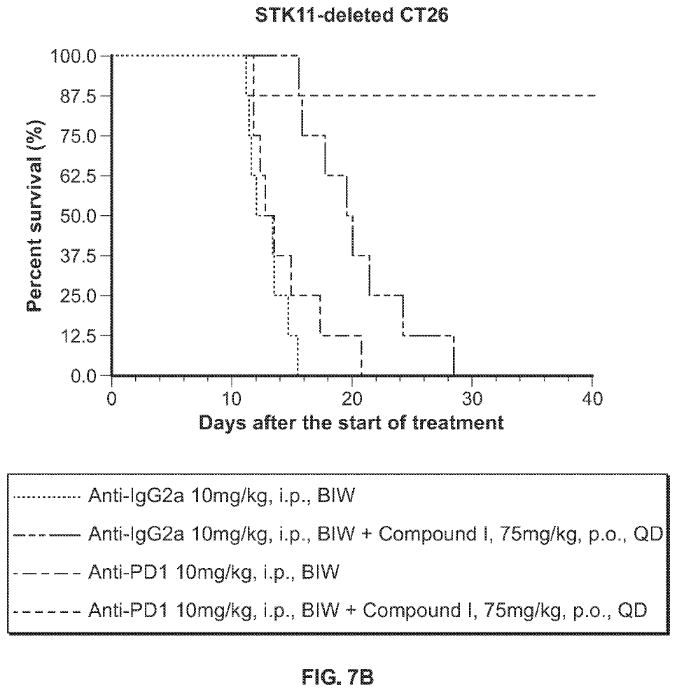

Briefly, mice were inoculated with STK11-deleted CT26 (murine colorectal carcinoma cell line) tumor model in a manner similar to that described in Example 1. The tumor model was resistant to murine anti-PD1 antibody by knockout of the STK11. Mice were treated with Compound I orally once daily and with anti-PD1, anti-IgG2a control, or anti-PD1 control twice per week as indicated. Tumor volume and survival was monitored over the course of treatment and plotted by individual animal for tumor volume (FIG. 7A) and by group for survival (FIG. 7B).

The results of Example 1 and Example 5 show that Compound I reverses resistance to immune checkpoint blockade driven by loss of STK11 in tumor models for colon adenocarcinoma and colorectal carcinoma.

### Example 6: Efficacy of Compound I in Combination with Anti-PD1 Antibody in Mouse Models with or without T Cells

The efficacy of Compound I in combination with anti-PD1 antibody in mouse models with or without T cells were determined in vivo.

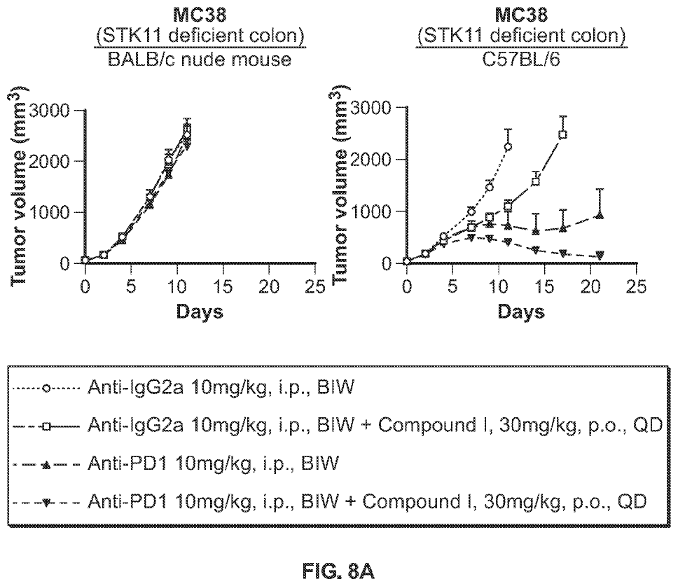

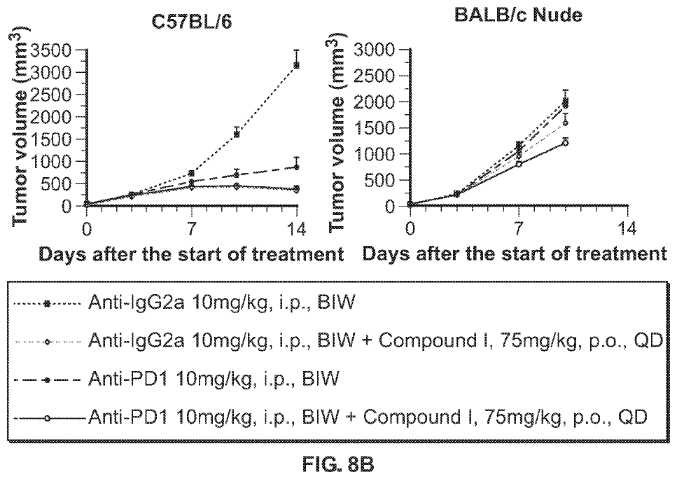

Briefly, athymic BALB/c Nude mice and C57BL/6 animals with STK11-deficient MC38 tumors were dosed orally once daily with Compound I at 30 mg/kg or 75 mg/kg alone or in combination with anti-PD1 twice per week (FIGS. 8A and 8B). The results show that the efficacy of Compound I with anti-PD1 requires an intact T cell compartment.

### Example 7: Cytokine Profiling of Tumors Treated with Compound I

The cytokine expression of tumors from mice treated with Compound I was determined by Nanostring PanCancer IO 360 analysis.

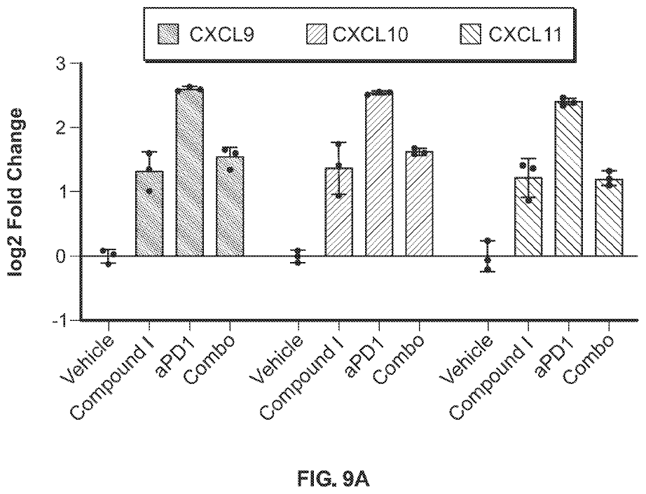

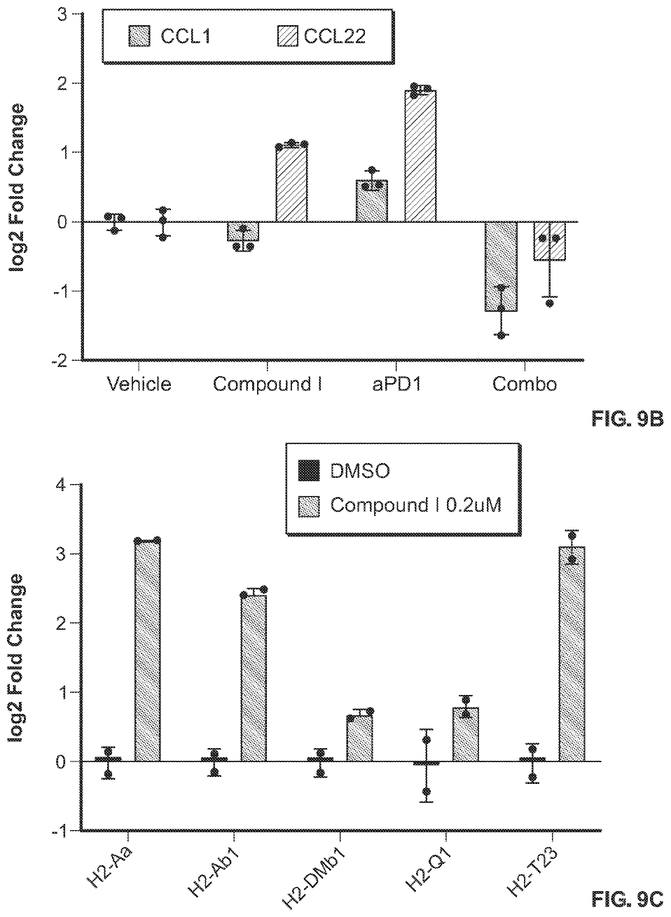

Briefly, STK11−/− MC38 tumors from mice treated for 7 days with 30 mg/kg of Compound I or anti-PD1 antibody alone or in combination were collected 8 hours post last dose. The gene expression profiles of CXCL9, CXCL10, and CXCL11 of the tumors were determined by Nanostring PanCancer IO 360 (FIG. 9A). Additionally, the gene expression profiles of Treg-recruiting chemokines CCL1 and CCL22 of the tumors were determined by Nanostring PanCancer IO 360 (FIG. 9B).

STK11-deficient MC38 cells were also profiled for HLA gene expression by Nanostring IO360 panel in vitro after 4 days of treatment with Compound I at 0.2 uM compared to DMSO solvent control (FIG. 9C).

The results show that HDAC1,2 selective inhibitor treatment drives expression of cytokines that promote anti-tumor activity. Additionally, Compound I treatment reverses immune evasion on tumor cells by changing expression of cytokines and antigen presentation genes.

### Example 8: T Cell Activity after Treatment with Compound I in Combination with Anti-PD1 Antibody

In this example, the ability of Compound I in combination with anti-PD1 antibody to increase T cell activity was determined.

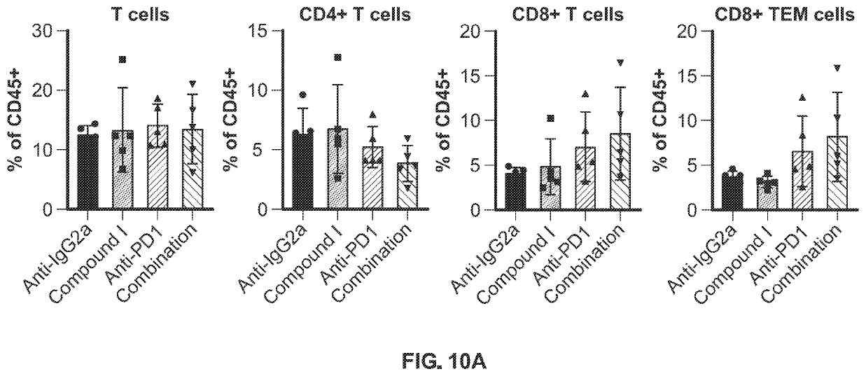

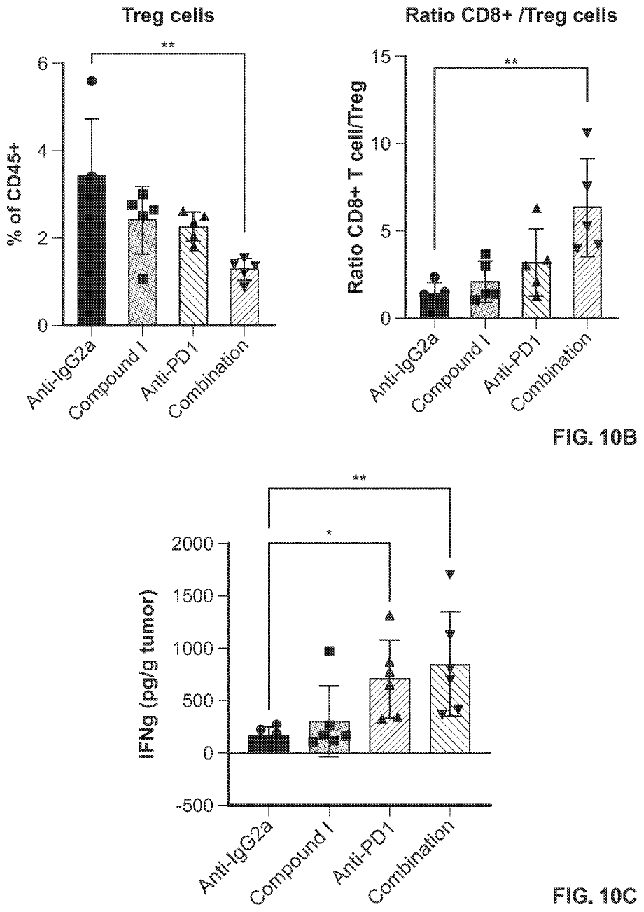

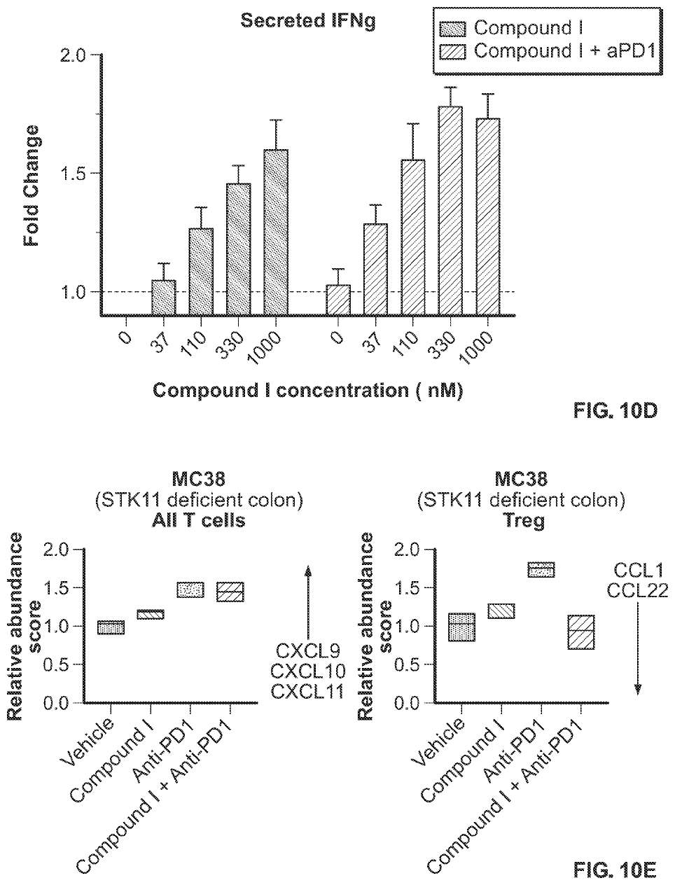

Briefly, mice with STK11-deleted MC38 tumors were treated for 7 days with 10 mg/kg of Compound I alone or in combination with anti-PD1 as described above. Tumor tissue was collected 8 hours post last dose and tumor infiltrating lymphocytes (TILs) were profiled by flow cytometry. T cell populations were analysed for total CD45+ cells, CD4+ cells, CD8+ cells, and CD8+ T effector memory (TEM) cells and Treg cells (FIG. 10A and FIG. 10B). Flow cytometry of Tregulatory (Treg) cells showed a significant decrease in frequency of Treg cells in the combination arm (FIG. 10B). The ratio of CD8+ T effector cells to T regulatory cells in each of the treatment groups showed a significantly increased ratio in the combination arm (FIG. 10B).

IFNγ levels were evaluated by Luminex analysis in tumors treated with 30 mg/kg of Compound I or in combination with anti-PD1 (FIG. 10C).

A co-culture of human NSCLC cells with PBMCs and fibroblasts was treated with a dose response of Compound I alone or in combination with a fixed dose of anti-PD1 for 72 hours. The IFNγ levels were quantified from tissue culture supernatant by ELISA (FIG. 10D).

The results show that the combination of Compound I and anti-PD1 antibody increased T cell activity in the tumor microenvironment and depleted Tregs.

To determine the immune correlates of response to Compound I combined with anti-PD-1 in the STK11-mutant tumor microenvironment, MC38_sgStk11 tumor-bearing mice were treated for 7 days with vehicle (5% DMA+30% PEG 400+65% of 30% HPβCD in water and 10 mg/kg anti-IgG2a), 30 mg/kg Compound I, 10 mg/kg anti-PD-1, or the combination. Tumors were harvested after 7 days and snap frozen for analysis using a gene expression panel for mouse tissue.

The total number of T cells was increased by anti-PD-1 alone and in combination with Compound I (FIG. 10E). The abundance of regulatory T cells (Tregs) was also increased by anti-PD-1. However, anti-PD-1+Compound I combination treatment prevented the increase in Tregs caused by anti-PD-1 treatment alone. STK11 loss-of-function mutation is known to be associated with primary resistance to anti-PD-1 treatment (Skoulidis et al 2018). These data demonstrate that Compound I and anti-PD-1 combination therapy decouples the recruitment of T effector cells and Tregs, leading to an increased ratio of effector/regulatory T cells that strongly favors immune cell-mediated tumor cell killing.

### Example 9: Analysis of Gene Regulation by Compound I

In this example, the gene expression changes between CoreDAC and other HDAC inhibitors was determined by Nanostring profiling.

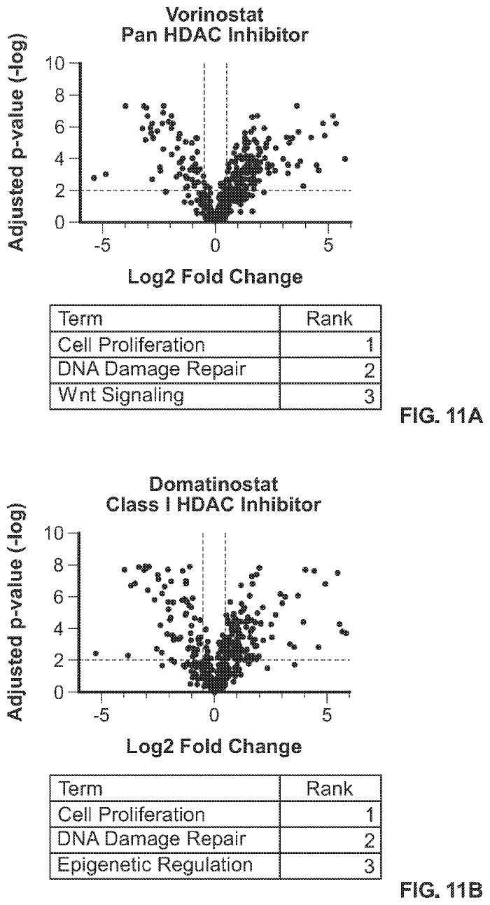

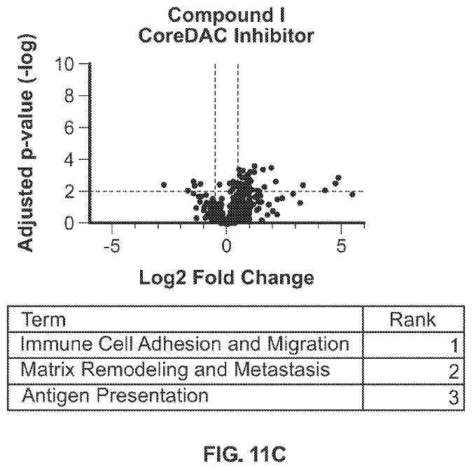

Briefly, A549 cells were treated for 96 hours in vitro with the HDAC inhibitors vorinostat, domatinostat and Compound I. To select comparable doses of each compound, an H3K9Ac AlphaLISA was performed, and the dose that increased H3K9Ac two-fold was chosen for each inhibitor. Cells were harvested and Nanostring profiling using the PanCancer IO360 panel was performed on the three treatment groups (FIGS. 11A-C). The top three ranked gene ontology groups for each compound as determined from the Nanostring data in (FIGS. 11A-C top panel) were determined using the nSolver software (FIGS. 11A-C bottom panel). The results show that Compound I regulates the expression of fewer genes than the less-selective HDAC inhibitors vorinostat and domatinostat.

### Example 10: Therapeutic Index and Cytotoxicity Analysis of Compound I

In this example, the toxicity and therapeutic index of Compound I was determined in vitro and in vivo.

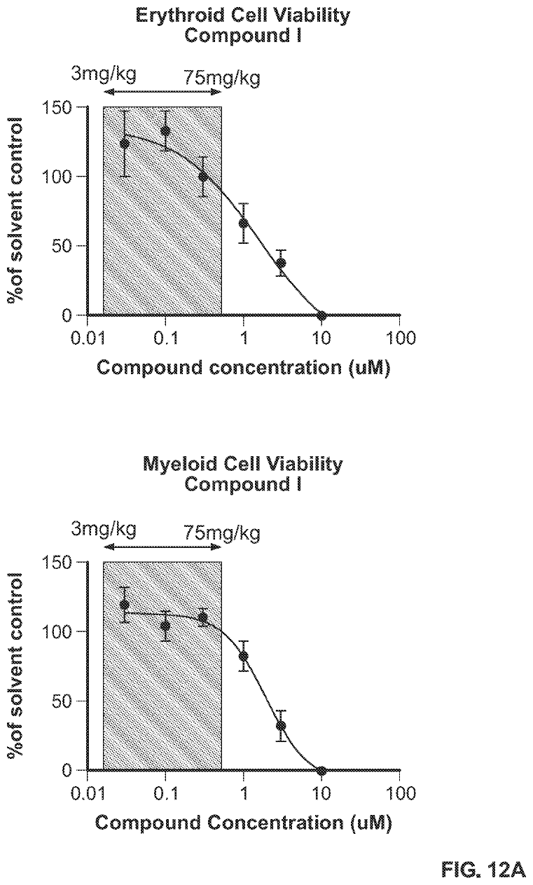

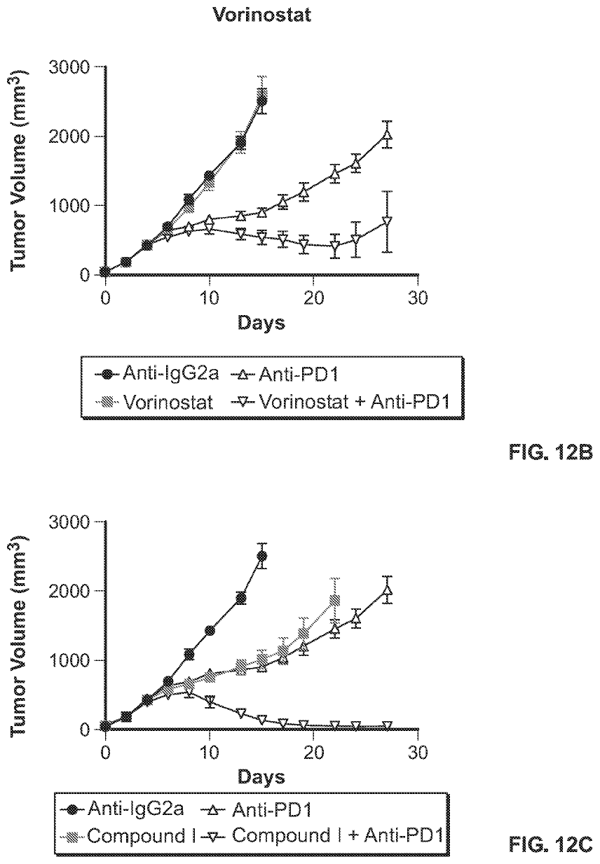

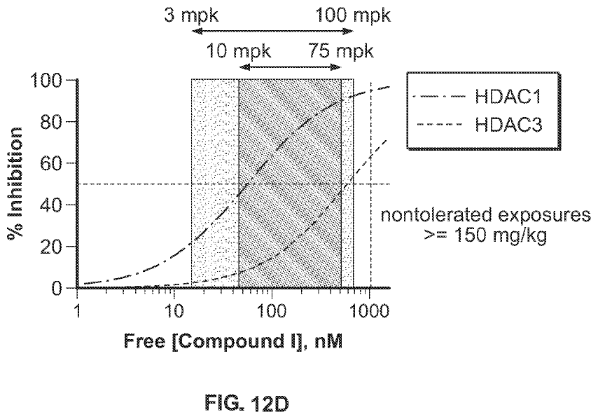

Briefly, a colony forming unit assay was performed in vitro to evaluate the impact of HDAC inhibitors on the viability of erythroid and myeloid cells. Cells were treated with a dose response of each compound for 14 days. Cell colonies were quantified at the end of the experiment and compared to a solvent control. The efficacious dose range of Compound I was also plotted (the range was between 3 mg/kg and 75 mg/kg) (FIG. 12A). The IC50s for each compound were calculated from the erythroid and myeloid colony formation unit assay. These IC50s were normalized to the compound's potency against HDAC1 in the cellular NanoBRET assay to allow for head-to-head compound comparison (Table 4-1).

IC50s from the erythroid and myeloid colony formation unit assay, normalized to the potency against HDAC1 (Table 4.1).

| | Erythroid/ | Myeloid/ |
|---|---|---|
| Treatment | HDAC1 IC50 | HDAC1 IC50 |
| Mocetinostat | 0.483 | 1.741 |
| Entinostat | 0.634 | 0.210 |
| Tucidinostat | 3.421 | 1.109 |
| TNG-0770701 | 13.673 | 15.282 |
| Compound I (CoreDAC) | 15.932 | 18.136 |

A clinically relevant dose of vorinostat for mouse was calculated using body surface area conversion. Mice with STK11-deleted MC38 tumors were treated in vivo with vorinostat or Compound I alone or vorinostat or Compound I in combination with anti-PD1. Tumor volumes were monitored over the course of treatment (FIG. 12B for vorinostat, FIG. 12C for Compound 1). The C_{average} of vorinostat or Compound I at doses used in FIG. 12B and FIG. 12C and the C_{average} coverage of the HDAC1 IC50 at that dose are shown in Table 4-2.

TABLE 4-2
C_{average} and IC50 of HDAC inhibitors

| Treatment | Cavg, u (uM) | Fold of HDAC1 IC50 |
|---|---|---|
| Vorinostat (pan) | 0.132* | 0.3 |
| Compound I (CoreDAC) | 0.192 | 3.5 |

Compound I concentrations to HDAC1 or HDAC3 inhibition in vivo were plotted in FIG. 12D. Shaded boxes indicate tolerated and efficacious dose ranges of Compound I. Non-tolerated exposures were over 150 mg/kg.

The results show that the Compound I is less cytotoxic and has an improved therapeutic index over less-selective HDAC inhibitors such as Vorinostat. The CoreDAC Compound I has an improved therapeutic index over previously developed HDAC inhibitors.

### Example 11: Pharmacological Properties of Compound I

The pharmacokinetics (PK) of Compound I in rats was determined following single IV bolus of 1 mg/kg in 20% wt/vol HPβCD, 1% vol/vol DMSO in saline or single PO dose of 3 mg/kg in 0.5% methylcellulose (MC) in water to male Sprague Dawley rats (fed for IV, fasted for PO). Plasma samples were collected from 3 animals/group at 0.05, 0.25, 0.5, 1, 2, 3, 4 8, and 24 hours after dosing. Selected PK parameters of Compound I are presented in Table 5. The PO bioavailability was 95.4%.

The PK of Compound I in beagle dogs was determined in plasma following single IV bolus and PO administration to non-naïve male and female beagle dogs. The vehicle used for IV was 20% wt/vol 2-hydroxypropyl β cyclodextrin (HPβCD), 1% vol/vol DMSO in saline, and PO was in 5% DMA, 30% PEG400 and 65% (30% HPβCD in water) (solution) or 0.5% MC (suspension). Plasma samples were collected from 3 animals/group at 0.083, 0.25, 0.5, 1, 3, 6, 9, 12, 24 and 48 hours after dosing. Selected PK parameters of Compound I are presented in Table 5. The PO bioavailability was 67.4% when Compound I was given as a solution at 3 mg/kg under fasted condition.

The PK of Compound I in male cynomolgus monkeys was determined in plasma following single IV bolus and PO administration to non-naïve male cynomolgus s monkeys The vehicle used for IV was 20% wt/vol HPβCD, 1% vol/vol DMSO in saline and for PO was in 0.5% MC (suspension). Plasma samples were collected from 3 animals/group at 0.083, 0.25, 0.5, 1, 3, 6, 9, 12, 24, and 48 hours after dosing. Selected PK parameters of Compound I are presented in Table 5. The PO bioavailability was 83% when Compound I was given as a suspension at 3 mg/kg under fed conditions.

The effects of Compound I on a cloned human ether-à-go-go-related gene (hERG) potassium channels stably expressed in human embryonic kidney (HEK) 293 cells were measured using manual patch-clamp technique. Briefly, Compound I was soluble at 100 uM in 0.3% DMSO, pH 6.9. A does range-finding (DRF) assay was performed with Compound I at 3. 30, and 100 uM concentrations. Compound I inhibited hERG currents by by 15.67%, 45.85%, and 73.57% at 3, 30, and 100 uM, respectively. The definitive hERG assays was used to detect the IC50 of Compound I. In the definite hERG assay, each concentration was measured in replicates of 3. The IC50 value for the inhibitory effect of Compound I on the hERG potassium current was 71.07 μM.

Drug metabolizing enzymes involved in the biotransformation of Compound I were investigated using human liver microsomes (HLM) and recombinant enzymes. Compound I was stable in HLMs (>93% remaining after 240 minutes of incubation with HLM protein concentration at 0.6 mg/ml). CYP selective inhibitor experiments were not performed due to lack of appreciable Compound I turnover. Compound I was minimally metabolized by recombinant human CYPs (rh CYPs; terminal half life (T_{1/2})>60 mins for CYP1A2, 2B6, 2C8, 2C9, 2C19, 2D6 and 3A4/5).

TABLE 5
Pharmacological properties of Compound I in rats, dogs, and monkeys
Pharmacological Properties

| | |
|---|---|
| MW, logD_{7.4}, TPSA | <400, 1.9, 96 |
| PO PK (rat, dog, cyno) % F | 95, 67, 83 |
| T_{1/2} (hr) | 4.8, 8.4, 6.8 |
| CL, p (mL/min/kg) | 7.7, 9.6, 9.4 |
| hERG IC₅₀ | >30 uM |
| CYP3A4 | No signal |

Abbreviations:
PO, oral;
PK, pharmacokinetics;
T ½, terminal half life;
CL, total body clearance;
hERG, human ether-à-go-go-related gene;

### Example 12: Predicted Human PK of Compound I

The human PK parameters for a dose of Compound I that is predicted to be efficacious were calculated based on the data from rodent and dog studies and are presented in Table 6.

TABLE 6
Predicted human PK parameters

| Vdss, p | 2.1 | L/kg | CL, p | 3.8 | mL/min/kg |
|---|---|---|---|---|---|
| % F | 78 | | MRT | 9.5 | hrs |
| T_{1/2} | 6.6 | hrs | Total AUC | 6.3 | ug*h/mL |

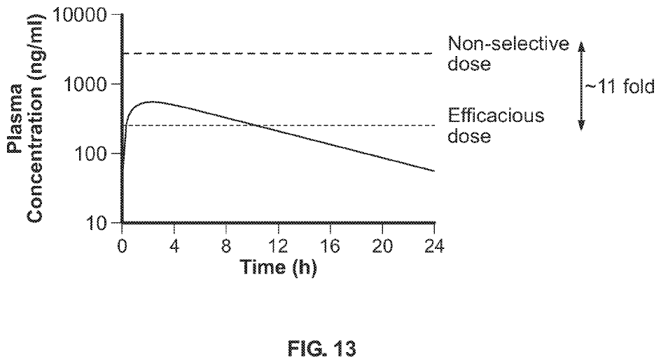

The predicted human pharmacokinetics and therapeutic window are modeled in FIG. 13.

### Example 13: Mouse PK/PD of Compound I

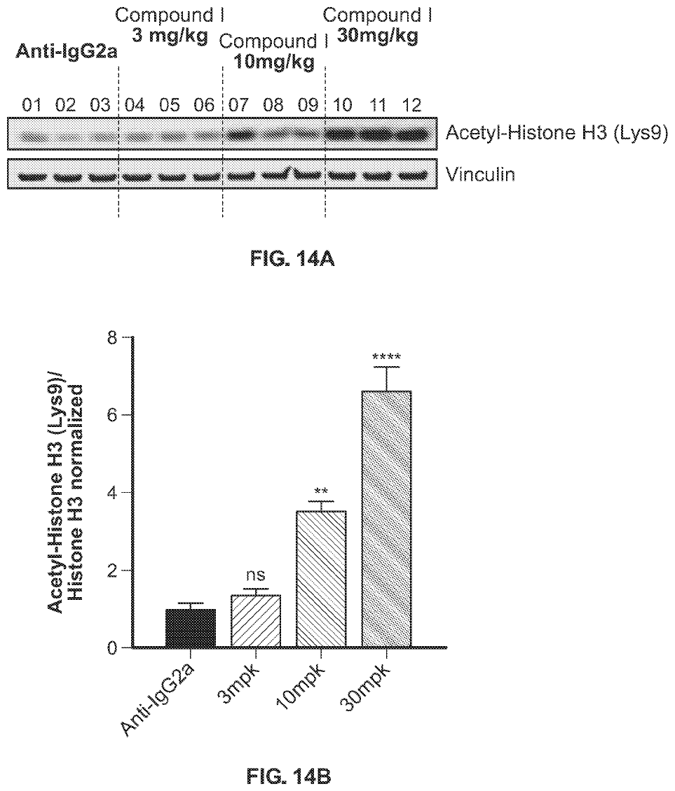

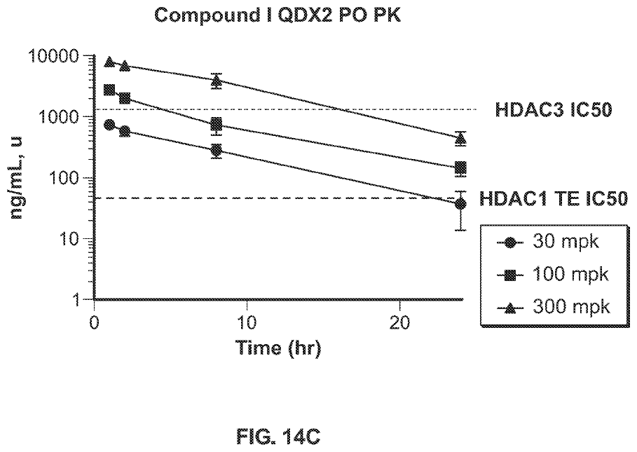

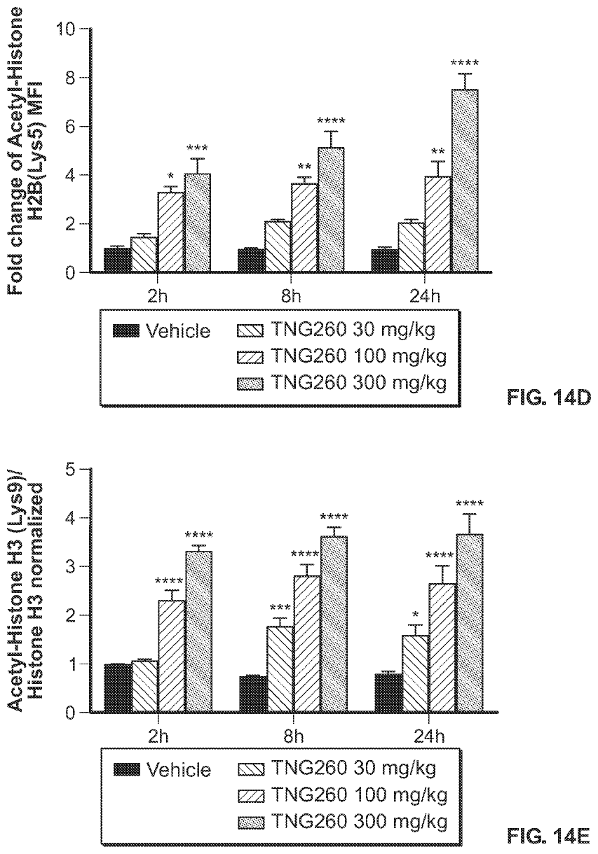

Formation and accumulation of acetylated histone 3 lysine 9 (H3K9Ac) in MC38 tumor tissue from mouse after treatment with Compound I was determined by Western blot. Briefly, female C57BL/6 mice with MC38 tumors were treated with Compound I for 7 days at the indicated dose (3 mg/kg, 10 mg/kg, and 30 mg/kg). Tissue was collected 8 hours post last dose and the H3K9Ac accumulation was quantified by Western blot in (FIG. 14A) and normalized to total histone H3 (FIG. 14B). The results show that incubation with Compound I leads to a dose dependent accumulation of H3K9Ac.

In a separate PK/PD study, MC38 tumor-bearing female C57BL/6 mice (12/group) were assigned to treatment groups with similar mean tumor volumes of approximately 430 mm³ and treated with vehicle (5% N,N-dimethylacetamide [DMA]+30% polyethylene glycol [PEG] 400+65% of 30% 2-hydroxypropyl-beta-cyclodextrin [HP-β-CD] in water) or Compound I at 30, 100, and 300 mg/kg once daily (QD) for 2 days. Plasma samples were collected at 1, 2, 8 and 24 hours after last dose and analyzed for Compound I concentrations. Peripheral blood mononuclear cells (PBMC) and tumor samples were harvested at 2, 8, and 24 hours after last dose. The PBMC samples were processed for determination of mean florescent level of acetyl-histone H2B by flow cytometry. Tumor samples were processed for determination of protein levels of acetyl-histone H3 by Western blot. No obvious body weight loss of more than 5% or adverse clinical signs were observed from Compound I treatment during this study.

Plasma concentrations of Compound I were dose proportional with maximal concentrations observed at 1 hour after the last dose (FIG. 14C). The free unbound exposures in plasma, based upon a 10.05% free fraction in C57BL/6 mouse plasma, were 5470, 17238, and 69665 h·ng/ml, respectively, for the 30, 100 and 300 mg/kg QD doses, indicating dose proportionality achieved within the dosing range. Compound I treatment at 30, 100, and 300 mg/kg QD resulted in a dose-dependent increase in tumor acetyl-histone protein levels, starting at 2 hours after the last dose. A maximal 8- and 3.7-fold induction of histone acetylation was observed, in PBMC and tumor, respectively, at 24 hours after the last dose following 300 mg/kg QD (2 days of treatment) of Compound I, as compared to vehicle control (FIG. 14D and FIG. 14E).

Overall, the PK/PD relationship was dose dependent, and the induction of histone acetylation was sustainable throughout the 24-hour treatment period when Compound I plasma concentrations remained above the in vitro IC50 of HDAC1.

### Example 14: Anti-Tumor Activity of Anti-PD-1 and Compound I Combination in a Colon Carcinoma Model with KRAS Mutation with STK11 KO (CT26 STK11KO) and without STK11 Alterations (Parental CT26 or Wild-Type CT26)

An in vivo anti-tumor efficacy of combination treatment of Anti-PD1 and Compound I in a CT26 mouse model was evaluated. CT26 is a colon carcinoma cell line with an endogenous KRAS G12D mutation. The experiment was performed in the parental/wild type line and in a line engineered with an STK11 knock-out (CT26_STK11KO) resulting in an anti-PD1 resistant model with lower CD8+ T cell infiltration.

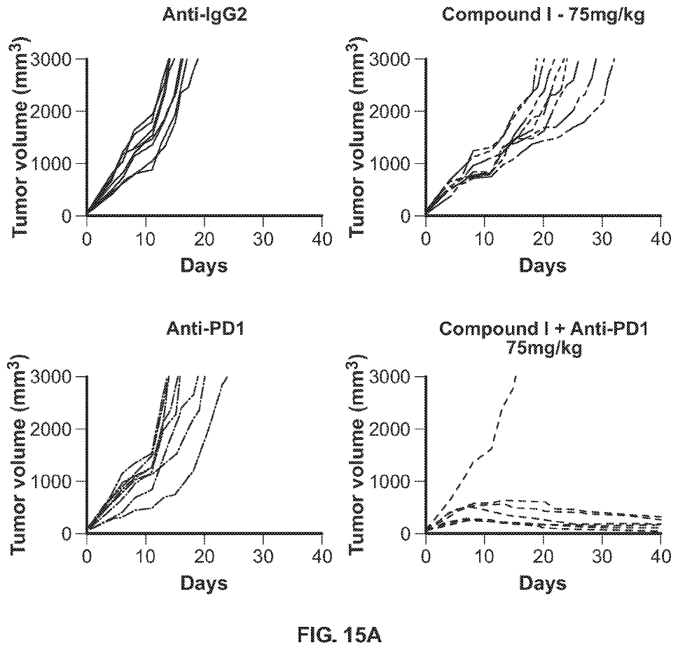

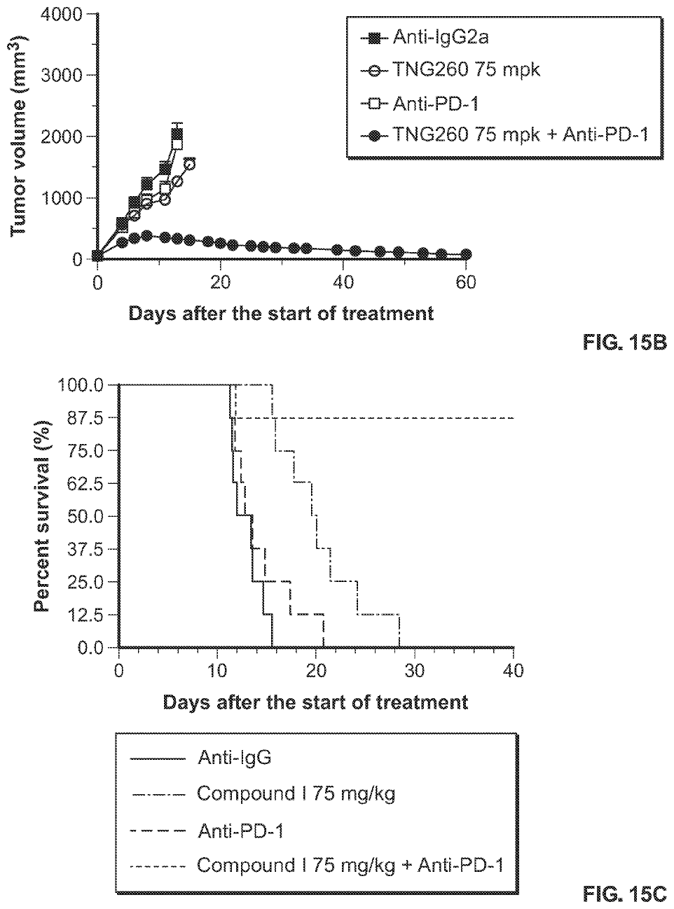

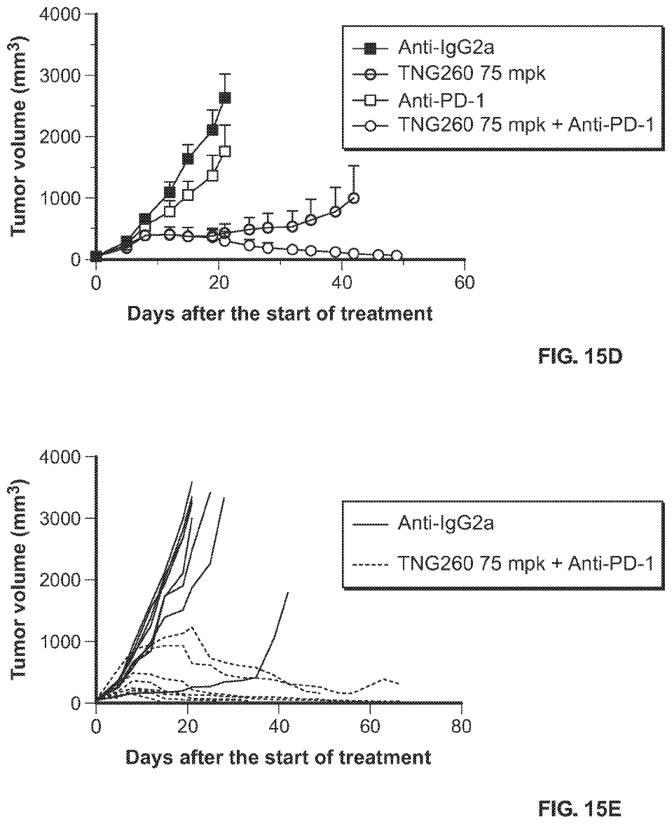

In each of the experiments, the animals (8/group) were divided into four groups. Group I was treated with a control antibody Anti-IgG2, group 2 was treated with Compound I 75 mg/kg, group 3 was treated with anti-PD1 antibody, and group 4 was treated with Compound I 75 mg/kg and anti-PD1 antibody. Tumor volume and survival was monitored over the course of treatment. For the CT26_STK11KO model, the tumor volume was plotted by individual animal (FIG. 15A) and by treatment group (FIG. 15B). Survival was plotted by treatment group (FIG. 15C).

For the CT26 parental model, the tumor volume was plotted by treatment group (FIG. 15D) and by individual animal for groups 1 and 4 (FIG. 15E).

The experiment shows that Compound I as a single agent has better efficacy in parental model (84% TGI) than in STK11KO model (39% TGI), whereas the combination treatment exhibits similar efficacy: ORR % of 86% (6/7) and 88% (7/8) in parental and STK11KO model, respectively.

### Example 15: Anti-Tumor Activity of Anti-CTLA4 and Compound I Combination in a Colon Carcinoma Model

An in vivo anti-tumor efficacy of combination treatment of anti-CTLA4 and Compound I in a CT26 mouse model was evaluated. CT26 is a colon carcinoma cell line with an endogenous KRAS G12D mutation. The experiment was performed in the parental/wild type line and in a line engineered with an STK11 knock-out (CT26_STK11KO) resulting in an anti-CTLA4-resistant model.

An in vivo anti-tumor efficacy of double combination treatment of Anti-CTLA4 and Compound I in a CT26 syngeneic model was performed. CT26 is a colon carcinoma cell line. The experiment was also performed with an STK11-null CT26 model, this model is CTLA4 resistant.

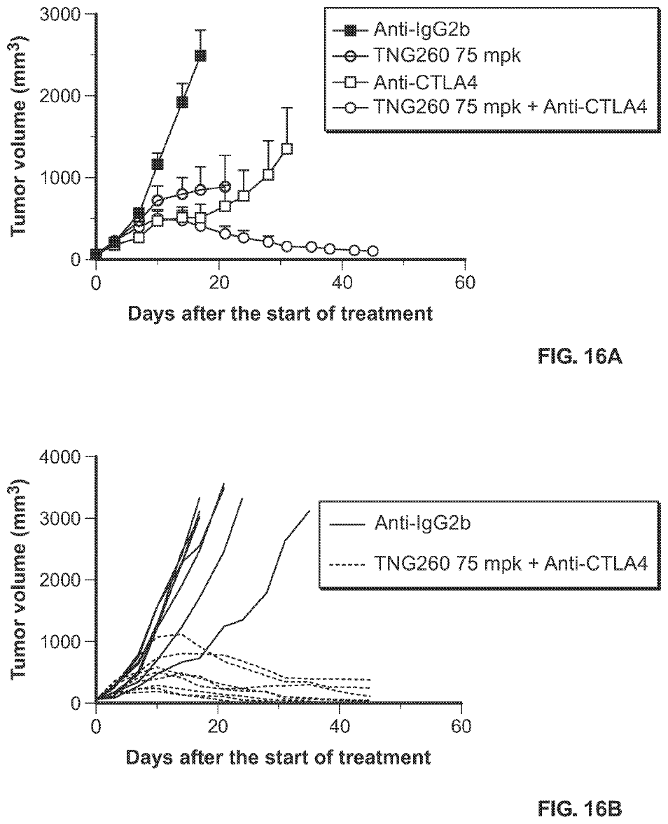

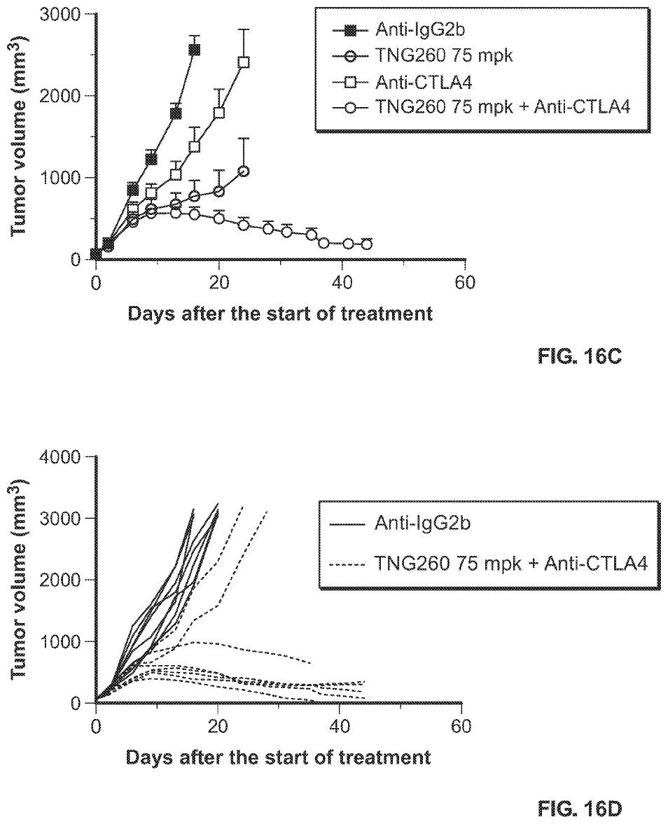

There were 8 mice per group. Group I was treated with a control antibody Anti-IgG2, group 2 was treated with Compound I 75 mg/kg, group 3 was treated with anti-CTLA4 antibody, and group 4 was treated with Compound I 75 mg/kg and anti-CTLA4 antibody. Tumor volume was monitored over the course of treatment. The tumor volume was plotted by treatment group (FIG. 16A), and the individual animals were plotted for group 1 and group 4 (FIG. 16B), for the parental model. The tumor volume was plotted by treatment group (FIG. 16C), and the individual animals were plotted for group 1 and group 4 (FIG. 16D), for the STK11 knockout model.

Compound I exhibited better efficacy in the parental model (84% TGI) than in STK11KO model (39% TGI). 100% of mice responded to the combination treatment in the parental model, while 75% (6/8) mice showed response in the STK11KO model.

Table 7 summarizes the results of the combination treatments from Examples 14 and 15.

TABLE 7
Summary of Compound I and checkpoint inhibitor
combination efficacy in CT26 models

| | CT26 Parental | | CT26 STK11KO | |
|---|---|---|---|---|
| | TGI % | ORR % | TGI % | ORR % |
| Anti-PD1 | 33 | 13 | 8 | 0 |
| Anti-CTLA4 | 80 | 25 | 46 | 13 |
| Compound I* | 84/66 | 63/63 | 39/70 | 0/38 |
| Compound I * + anti-PD1 | 88 | 100 | 73 | 88 |
| Compound I * + anti-CTLA4 | 84 | 100 | 68 | 75 |

* Compound I 75 mg/kg QD dosing

### Example 16: Anti-Tumor Activity of Anti-PD-1 and Compound I Combination in a Lewis Lung Carcinoma Model

An in vivo anti-tumor efficacy of double combination treatment of Anti-PD1 and Compound I in a PD-1 resistant STK11-null 3LL model was performed. The parental (non STK11KO) 3LL line is relatively resistant to anti-PD1 treatment.

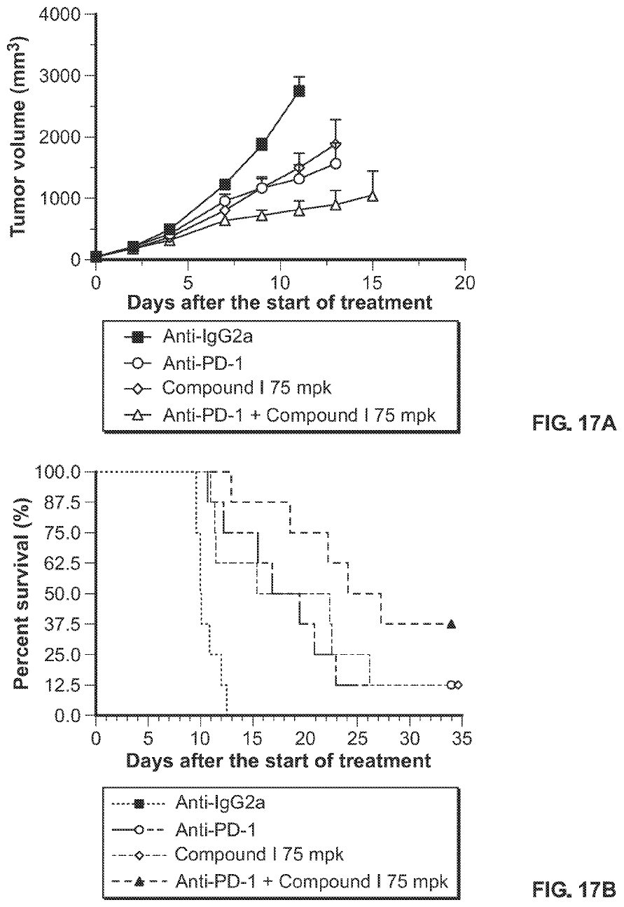

The animals (8/group) were divided into 4 groups: Group I was treated with a control antibody Anti-IgG2, group 2 was treated with anti-PD1 antibody, group 3 was treated with Compound I 75 mg/kg, and group 4 was treated with Compound 1 75 mg/kg and anti-PD1 antibody. Tumor volume and survival was monitored over the course of treatment. The tumor volume and survival was plotted by treatment group (FIG. 17A and FIG. 17B, respectively). The combination treatment with Compound I decreased the PD-1 resistance of this model caused by the SKT11 knockout to the baseline characteristic of the parental line as shown by Group 4's decreased tumor volume and increased survival over time compared to the other groups.

The median Time-To-Event (TTE and P values compared to control group 1 (Vehicle) are summarized in Table 8.

TABLE 8
Median TTE of PD-1 resistant STK11-null 3LL
model groups of the indicated treatment

| | Vehicle | Anti-PD1 | TNG260 | Combo |
|---|---|---|---|---|
| Median TTE (Days) | 10 | 19 | 18 | 26 |
| P vs. Vehicle | — | 0.0007 | 0.003 | <0.0001 |

1. An HDAC inhibitor for use in a method of treating a subject having, or at risk of developing, a cancer, the method comprising administering to the subject an effective amount of a histone deacetylase (HDAC) inhibitor, wherein the cancer is identified as having modified STK11 activity or expression.

2. The HDAC inhibitor for use of claim 1, wherein the histone deacetylase inhibitor is administered in combination with one or more additional therapeutic agents.

3. The HDAC inhibitor for use of claim 2, wherein at least one of the additional therapeutic agents is an immune checkpoint modulator.

4. An HDAC inhibitor for use in a method of treating a cancer in a subject comprising administering to the subject an immune checkpoint modulator and an HDAC inhibitor, wherein the treatment modulates and/or improves the Teff cell to Treg cell ratio in the tumor or tumor microenvironment, wherein the cancer is identified as having modified STK11 activity or expression.

5. An HDAC inhibitor for use in a method of treating a cancer in a subject comprising administering to the subject an immune checkpoint modulator and an HDAC inhibitor, wherein the treatment reduces or depletes Treg cells in the tumor or tumor microenvironment, wherein the cancer is identified as having modified STK11 activity or expression.

6. An HDAC inhibitor for use in a method of treating a cancer in a subject comprising administering to the subject an immune checkpoint modulator and an HDAC inhibitor, wherein the treatment induces or increases the expression of cytokines that promote anti-tumor activity, wherein the cancer is identified as having modified STK11 activity or expression.

7. The HDAC inhibitor for use in a method of claim 6, wherein the cytokines are selected from the group of CXCL9, CXCL10, and CXCL 11.

8. An HDAC inhibitor for use in a method of treating a cancer in a subject comprising administering to the subject an immune checkpoint modulator and an HDAC inhibitor, wherein the treatment reduces the expression of cytokines that promote Treg cell recruitment, wherein the cancer is identified as having modified STK11 activity or expression.

9. The HDAC inhibitor for use in the method of claim 8, wherein the cytokines are CCL1 or CCL22.

10. An HDAC inhibitor for use in a method of treating a cancer in a subject comprising administering to the subject an HDAC inhibitor, wherein the administering of the HDAC inhibitor does not reduce erythroid or myeloid cell viability, wherein the cancer is identified as having modified STK11 activity or expression.

11. An HDAC inhibitor for use in a method of treating cancer in a subject, wherein the cancer presents an immune evasion phenotype characterized by STK11 mutant expression comprising: administering an HDAC1,2 selective inhibitor, wherein the HDAC1,2 selective inhibitor is capable of attenuating or reversing the immune evasion phenotype.

12. The HDAC inhibitor for use in a method of claim 10 or 11, further comprising administering an immune checkpoint modulator.

13. An HDAC inhibitor for use in a method of treating a cancer in a subject comprising administering to the subject an HDAC inhibitor, wherein an immune checkpoint modulator has been, is, or will be administered to the subject, wherein the cancer is identified as having modified STK11 activity or expression.

14. An HDAC inhibitor for use in a method of treating a cancer in a subject comprising administering to the subject an immune checkpoint modulator, wherein an HDAC inhibitor has been, is, or will be administered to the subject, wherein the cancer is identified as having modified STK11 activity or expression.

15. The HDAC inhibitor for use of claim 1, wherein the HDAC inhibitor is administered in combination with two or more additional therapeutic agents, wherein at least two of the additional therapeutic agents are immune checkpoint modulators.

16. The HDAC inhibitor for use of any one of claims 3-9 and 12-15, wherein each immune checkpoint modulator is independently a checkpoint inhibitor, a T cell co-stimulatory receptor agonist or a dendritic cell co-stimulatory receptor agonist.

17. The HDAC inhibitor for use of any one of claims 3-9 and 12-15, wherein at least one immune checkpoint modulator is independently a T cell co-stimulatory receptor agonist or a dendritic cell co-stimulatory receptor agonist.

18. The HDAC inhibitor for use of any one of claims 3-9 and 12-15, wherein at least one immune checkpoint modulator is a checkpoint inhibitor.

19. The HDAC inhibitor for use of claim 18, wherein each checkpoint inhibitor is independently selected from an anti-CTLA-4 agent, an anti-PD-1 agent, an anti-PD-L1 agent, an anti-4-1 BB agent, an anti-OX-40 agent, an anti-GITR agent, an anti-CD27 agent, an anti-CD28 agent, an anti-CD40 agent, an anti-LAG3 agent, an anti-ICOS agent, an anti-TWEAKR agent, an anti-HVEM agent, an anti-TIM-1 agent, an anti-TIM-3 agent, an anti-VISTA agent, and an anti-TIGIT agent.

20. The HDAC inhibitor for use of claim 18, wherein each checkpoint inhibitor is independently selected from an anti-CTLA-4 agent, an anti-PD-1 agent and an anti-PD-L1 agent.

21. The HDAC inhibitor for use of claim 18, wherein the checkpoint inhibitor is an anti-CTLA-4 agent.

22. The HDAC inhibitor for use of claim 18, wherein the checkpoint inhibitor is an anti-PD1 agent.

23. The HDAC inhibitor for use of claim 18, wherein the checkpoint inhibitor is an anti-PD-L1 agent.

24. The HDAC inhibitor for use of claim 1, wherein the HDAC inhibitor is administered in combination with an anti-CTLA-4 agent and an anti PD-1 or anti PD-L1 agent.

25. The HDAC inhibitor for use of any one of claims 3-9 and 12-24, wherein each immune checkpoint inhibitor is independently an antibody.

26. The HDAC inhibitor for use of claim 25, wherein the each checkpoint inhibitor is independently selected from an anti-CTLA-4 antibody, an anti-PD-1 antibody, an anti-PD-L1 antibody, an anti-4-1 BB antibody, an anti-OX-40 antibody, an anti-GITR antibody, an anti-CD27 antibody, an anti-CD28 antibody, an anti-CD40 antibody, an anti-LAG3 antibody, an anti-ICOS antibody, an anti-TWEAKR antibody, an anti-HVEM antibody, an anti-TIM-1 antibody, an anti-TIM-3 antibody, an anti-VISTA antibody, and an anti-TIGIT antibody.

27. The HDAC inhibitor for use of claim 25, wherein each checkpoint inhibitor is independently selected from an anti-CTLA-4 antibody, an anti-PD-1 antibody and an anti-PD-L1 antibody.

28. The HDAC inhibitor for use of claim 25, wherein each immune checkpoint inhibitor is independently selected from nivolumab; CT-011; AMP-224; pembrolizumab; pidilizumab; cemiplimab; dostarlimab; prolgolimab; spartalizumab; camrelizumab; sasanlimab, sintilimab; tislelizumab; toripalimab; retifanlimab; MEDI0680; budigalimab and geptanolimab.

29. The HDAC inhibitor for use of claim 25, wherein each checkpoint inhibitor is independently selected from an anti-PD1 antibody and an anti-PD-L1 antibody.

30. The HDAC inhibitor for use of claim 25, wherein the checkpoint inhibitor is an anti-CTLA-4 antibody.

31. The HDAC inhibitor for use of claim 25, wherein the checkpoint inhibitor is an anti-PD1 antibody.

32. The HDAC inhibitor for use of claim 25, wherein the checkpoint inhibitor is an anti-PD1-L1 antibody.

33. The HDAC inhibitor for use of claims 26, 27 and 30, wherein the anti-CTLA-4 antibody is ipilimumab.

34. The HDAC inhibitor for use of any one of claims 26, 27, 29 and 31, wherein the anti-PD-1 antibody is pembrolizumab or nivolumab.

35. The HDAC inhibitor for use of any one of claims 26, 27, 29 and 31, wherein the anti-PD-1 antibody is pembrolizumab.

36. The HDAC inhibitor for use of any one of claims 26, 27, 29 and 31, wherein the anti-PD-1 antibody is nivolumab.

37. The HDAC inhibitor for use of any one of claims 26, 27, 29 and 32, wherein the anti-PD-L1 antibody is atezolizumab (CAS number 1380723-44-3), avelumab (CAS number 1537032-82-8), or durvalumab (CAS number 1428935-60-7).

38. The HDAC inhibitor for use of any one of claims 3-9 and 12-24, wherein the immune checkpoint modulator is a T cell co-stimulatory receptor agonist or a dendritic cell co-stimulatory receptor agonist.

39. The HDAC inhibitor for use of any one of claims 2-9 and 12-38, wherein at least one additional therapeutic agent is targeted agent.

40. The HDAC inhibitor for use of claim 39, wherein each targeted agent is independently selected from an anti-angiogenesis agent (e.g., an anti-VEGF agent), a KRAS inhibitor, an ALK inhibitor, a ROS1 inhibitor, a BRAF inhibitor, a RET inhibitor, a MEK inhibitor, a MET inhibitor and a TRK inhibitor.

41. The HDAC inhibitor for use of claim 39, wherein each targeted agent is independently selected from bevacizumab, ramucirumab, sotorasib, crizotinib, ceritinib, alectinib, brigatinib, lorlatinib, entrectinib, dabrafenib, trametinib, capmatinib, tepotinib and larotrectinib.

42. The HDAC inhibitor for use of any one of claims 3-9 and 12-41, wherein at least one additional therapeutic agent is a chemotherapeutic agent.

43. The HDAC inhibitor for use of claim 42, wherein each chemotherapeutic agent is independently selected from cisplatin, carboplatin, paclitaxel, Albumin-bound paclitaxel (nab-paclitaxel), docetaxel, gemcitabine, vinorelbine, etoposide and pemetrexed.

44. The HDAC inhibitor for use of claim 42, wherein at least one chemotherapeutic agent is a platinum-containing therapeutic agent.

45. The HDAC inhibitor for use of claim 42, wherein one chemotherapeutic agent is a platinum-containing chemotherapeutic agent (e.g., cisplatin) and a second chemotherapeutic agent is pemetrexed.

46. The HDAC inhibitor for use of any one of claims 3-9 and 12-45, wherein at least one additional therapeutic agent is radiation.

47. The HDAC inhibitor for use of any one of claims 1-46, wherein the cancer is resistant to anti-PD1 therapy or anti-PD-L1 therapy.

48. The HDAC inhibitor for use of any one of claims 1-47, wherein the cancer is resistant to chemotherapy (e.g., platinum-containing chemotherapy).

49. The HDAC inhibitor for use of any one of claims 1-48, wherein the cancer does not respond to or benefit from treatment with an immune checkpoint modulator when administered alone or as part of a treatment regimen that does not include an HDAC inhibitor.

50. The HDAC inhibitor for use of any one of claims 1-49, wherein the cancer is selected from the group consisting of: lung cancer (e.g., lung adenocarcinoma, non-small cell lung cancer (NSCLC), squamous cell lung carcinoma), colorectal cancer (e.g., colon adenocarcinoma, rectal adenocarcinoma), breast cancer (e.g., invasive ductal carcinoma), pancreatic cancer (e.g., pancreatic adenocarcinoma), endometrial cancer (e.g., endometrioid carcinoma), neuroendocrine cancer (e.g., large cell neuroendocrine carcinoma), melanoma, non-melanoma skin cancer (e.g., skin squamous cell carcinoma), cholangiocarcinoma, gallbladder cancer, ovarian cancer (e.g., ovarian serous adenocarcinoma), bladder cancer (e.g., bladder urothelial carcinoma), prostate cancer (e.g., prostate adenocarcinoma), cervical cancer, endocervical cancer or cancer of unknown primary (e.g., adenocarcinoma of unknown primary).

51. The HDAC inhibitor for use of claim 50, wherein the cancer is lung cancer.

52. The HDAC inhibitor for use of claim 51, wherein the cancer is lung adenocarcinoma.

53. The HDAC inhibitor for use of claim 51, wherein the cancer is non-small cell lung cancer (NSCLC).

54. The HDAC inhibitor for use of claim 53, wherein the cancer is non-squamous non-small cell lung cancer (NSCLC).

55. The HDAC inhibitor for use of claim 50, wherein the cancer is colorectal cancer or colon adenocarcinoma.

56. The HDAC inhibitor for use of any one of claims 1-55, wherein the cancer has decreased STK11 expression.

57. The HDAC inhibitor for use of any one of claims 1-55, wherein the cancer has a STK11 mutation.

58. The HDAC inhibitor for use of any one of claims 1-55, wherein the cancer is identified as having a STK11 mutation and one or more additional mutations.

59. The HDAC inhibitor for use of claim 58, wherein the additional mutations are selected from KRAS mutations and KEAP1 mutations.

60. The HDAC inhibitor for use of claim 58, wherein the additional mutations are KRAS mutations.

61. The HDAC inhibitor for use of claim 59 or 60, wherein the KRAS mutations are mutations at position G12, optionally wherein the KRAS mutations are selected from G12D mutations, G12C mutations, G12V mutations or combinations thereof.

62. The HDAC inhibitor for use of claim 59, wherein the additional mutations are KEAP1 mutations.

63. The HDAC inhibitor for use of claim 59, wherein the additional mutations are KRAS mutations and KEAP1 mutations.

64. The HDAC inhibitor for use of any one of claims 57-63, wherein the STK11 mutation is an inactivating (loss of function) mutation.

65. The HDAC inhibitor for use of any one of claims 1-64, wherein the histone deacetylase inhibitor is a selective inhibitor of histone deacetylase 1 (HDAC1-selective inhibitor).

66. The HDAC inhibitor for use of any one of claims 1-64, wherein the histone deacetylase inhibitor is a selective inhibitor of histone deacetylase 1 and histone deacetylase 2 (HDAC1,2-selective inhibitor).

67. The HDAC inhibitor for use of any one of claims 1-64, wherein the histone deacetylase inhibitor is a selective HDAC Class I inhibitor.

68. The HDAC inhibitor for use of any one of claims 1-67, wherein the histone deacetylase inhibitor is a CoREST-selective deacetylase inhibitor.

69. A method for ascertaining susceptibility of a subject having or having been diagnosed with cancer to treatment with an HDAC inhibitor, the method comprising: determining: i) the presence or absence of a STK11 mutation; and/or ii) the level of STK11 activity or expression in the subject or a sample derived from the subject: wherein the presence of a STK11 mutation and/or a modified level of STK11 activity or expression is indicative of susceptibility to treatment with an HDAC inhibitor.

70. The HDAC inhibitor for use of any one of claims 1-68 wherein the HDAC inhibitor is a compound of Formula (I) 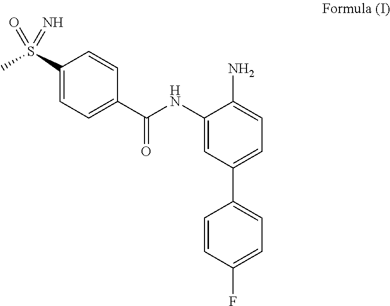 [SMILES: C[S@@](=N)(=O)c1ccc(C(=O)Nc2cc(-c3ccc(F)cc3)ccc2N)cc1]
or a pharmaceutically acceptable salt thereof.

71. A method for ascertaining susceptibility of a subject having or having been diagnosed with cancer to treatment with a combination of an HDAC inhibitor and an immune checkpoint modulator, the method comprising: determining: i) the presence or absence of a STK11 mutation; and/or ii) the level of STK11 activity or expression in the subject or a sample derived from the subject; wherein the presence of a STK11 mutation and/or a modified level of STK11 activity or expression is indicative of susceptibility to treatment with a combination of an HDAC inhibitor and an immune checkpoint modulator.

72. A method for ascertaining susceptibility of a subject having or having been diagnosed with cancer to use of an HDAC inhibitor in a method of treating of any one of claims 1-70, the method for ascertaining comprising: determining: i) the presence or absence of a STK11 mutation; and/or ii) the level of STK11 activity or expression in the subject or a sample derived from the subject; wherein the presence of a STK11 mutation and/or a modified level of STK11 activity or expression is indicative of susceptibility to use of an HDAC inhibitor in a method of treating of any one of claims 1-70.
